(19) United States
(12) Patent Application Publication
(10) Pub. No.: US 2015/0320675 A1
(43) Pub. Date: Nov. 12, 2015

(54) Modified Release Osmotic Pump for PH-Responsive Drug Delivery

(71) Applicant: UNIVERSITY OF UTAH RESEARCH FOUNDATION, Salt Lake City, UT (US)

(72) Inventors: Patrick F. Kiser, Chicago, IL (US); Rachna A. Rastogi, Salt Lake City, UT (US)

(21) Appl. No.: 14/655,643

(22) PCT Filed: Jan. 17, 2014
(86) PCT No.: PCT/US14/12081

§ 371 Date: Jun. 25, 2015

Related U.S. Application Data
(60) Provisional application No. 61/849,097, filed on Jan. 18, 2013.

(51) Int. Cl. A61K 9/00; A61K 9/28; A61K 35/747; A61K 31/513

(52) U.S. Cl. CPC A61K 9/0004; A61K 9/0036; A61K 31/513; A61K 9/2866; A61K 35/747

(57) ABSTRACT

In one aspect, the invention relates to controlled-release devices and methods of using same, alone or in combination with other agents, to deliver pharmaceutical agents, and more particularly to deliver birth control agent, fertility agents, hormone replacement agents, cervical ripening agents, agents to prevent a sexually transmitted disease, agents to treat a sexually transmitted disease, biologics, agents to treat a mucosal infection, agents to treat uterine fibroids, agents to treat reproductive cancers, agents to treat nausea gravidarum, and/or agents to treat endometriosis. This abstract is intended as a scanning tool for purposes of searching in the particular art and is not intended to be limiting of the present invention.

### CROSS-REFERENCE TO RELATED APPLICATIONS

This application claims the benefit of U.S. Provisional Application No. 61/849,097, filed on Jan. 18, 2013, which is incorporated herein by reference in its entirety.

### STATEMENT REGARDING FEDERALLY SPONSORED RESEARCH

This invention was made with government support under Grant No. U19 AI 076980 awarded by the National Institutes of Health (NIH). The United States government has certain rights in the invention.

### BACKGROUND

The vaginal drug delivery system (DDS) portfolio is dominated by short-acting formulations and long-acting intravaginal rings. Currently, the field lacks delivery systems that can be used “on-demand” but with durations between that of daily and monthly. While “on-demand” vaginal tablets can be an alternative with higher use acceptance compared to polymers (Minkin, M. J., et al. (2013) Int. J. Womens Health 5, 133-139; Rioux, J. E., et al. (2000) Menopause 7, 156-161), they have been less explored as HIV prevention technology platforms. Vaginal tablets can be manufactured easily using standard tableting equipment, are suitable for formulation of water-sensitive drugs, and can have long term stability without cold-chain storage requirements (Adams and Kashuba (2012) Best Pract. Res. Clin. Obstet. Gynaecol. 26, 451-462). However, a common problem among conventional vaginal tablets and polymers is the short duration of pharmacokinetics (PK) for most drugs (an exception is Class I drugs (Amidon, G. L., et al. (1995) Pharm. Res. 12, 413-420) with high intracellular half-lives); therefore, they require repeated dosing to ensure consistently protective drug levels. Frequent administration and the potentially associated low adherence ultimately may impact the performance of topical PrEP agents in clinical trials (Marrazzo, J., et al. (2013) 20^{th} Conference on Retroviruses and Opportunistic Infections, Atlanta, Ga., pp. 26 LB). Thus, there remains a need for new methods of antiretroviral drug delivery systems that can be used “on-demand.”

### SUMMARY

In accordance with the purpose(s) of the invention, as embodied and broadly described herein, the invention, in one aspect, relates to a controlled-release device comprising: a) a core comprising: i) a water-swellable gel-forming polymer; and ii) optionally, an osmotic agent; and iii) optionally, a pharmacologically active agent; b) a substantially inelastic, water-insoluble coating substantially enclosing the core, wherein at least a portion of the coating comprises a semipermeable membrane; and c) at least one orifice in the coating, wherein the orifice is positioned and dimensioned to allow controlled-release of the polymer from the core in response to swelling of the polymer, wherein the core comprises no more than 15 wt % of the osmotic agent.

Also disclosed are controlled-release devices comprising: a) a core comprising: i) a water-swellable gel-forming polymer and/or an osmotic agent; and ii) optionally, a pharmacologically active agent; and b) a water-insoluble coating substantially enclosing the core, wherein at least a portion of the coating comprises a pH-responsive material.

Also disclosed are controlled-release devices comprising: a) a core comprising: i) a water-swellable gel-forming polymer and/or an osmotic agent; and ii) at least one lubricant; b) a substantially inelastic, water-insoluble coating substantially enclosing the core, wherein at least a portion of the coating comprises a semipermeable membrane; and c) at least one orifice in the coating, wherein the orifice is positioned and dimensioned to allow controlled-release of the lubricant from the core.

Also disclosed are controlled-release devices comprising: a) a core comprising: i) a water-swellable gel-forming polymer and/or an osmotic agent; and ii) at least one biologic; b) a substantially inelastic, water-insoluble coating substantially enclosing the core, wherein at least a portion of the coating comprises a semipermeable membrane; and c) at least one orifice in the coating, wherein the orifice is positioned and dimensioned to allow controlled-release of the biologic from the core.

Also disclosed are methods of making a controlled-release device, the method comprising the steps of: a) providing a core comprising a water-swellable gel-forming polymer, optionally, an osmotic agent, and, optionally, a pharmacologically active agent; b) substantially enclosing the core within a substantially inelastic, water-insoluble coating, wherein at least a portion of the coating comprises a semipermeable membrane; and c) creating at least one orifice in the coating, wherein the core comprises no more than 15 wt % of the osmotic agent.

Also disclosed are methods of administering a lubricant, methods of preventing fertility, methods of promoting fertility, methods of preventing a sexually transmitted disease, methods of treating a sexually transmitted disease, methods of hormone replacement, methods of cervical ripening, methods of treating a mucosal infection, methods of delivering a biologic, methods of treating vaginal dryness, methods of treating uterine fibroids, methods of treating reproductive cancers, methods of treating nausea gravidarum, and methods of treating endometriosis.

Also disclosed are methods of delivering a pharmacologically active agent, the method comprising contacting a controlled-release device with vaginal membrane tissue, wherein the device comprises: a) a core comprising: i) a water-swellable gel-forming polymer and/or an osmotic agent; and ii) an effective amount of a pharmacologically active agent; b) a substantially inelastic, water-insoluble coating substantially enclosing the core, wherein at least a portion of the coating comprises a semipermeable membrane; and c) at least one orifice in the coating, wherein the orifice is positioned and dimensioned to allow controlled-release of the pharmacologically active agent from the core.

Also disclosed are kits comprising a controlled-release device, wherein the pharmacologically active agent is present, and at least one of: a) a second pharmacologically active agent; b) a second controlled-release device comprising a second pharmacologically active agent; c) an applicator; and d) instructions for contacting mucous membrane tissue of a mammal.

While aspects of the present invention can be described and claimed in a particular statutory class, such as the system statutory class, this is for convenience only and one of skill in the art will understand that each aspect of the present invention can be described and claimed in any statutory class. Unless otherwise expressly stated, it is in no way intended that any method or aspect set forth herein be construed as requiring that its steps be performed in a specific order. Accordingly, where a method claim does not specifically state in the claims or descriptions that the steps are to be limited to a specific order, it is no way intended that an order be inferred, in any respect. This holds for any possible non-express basis for interpretation, including matters of logic with respect to arrangement of steps or operational flow, plain meaning derived from grammatical organization or punctuation, or the number or type of aspects described in the specification.

### BRIEF DESCRIPTION OF THE FIGURES

The accompanying figures, which are incorporated in and constitute a part of this specification, illustrate several aspects and together with the description serve to explain the principles of the invention.

FIG. 1 shows a representative diagram illustrating a cross-sectional view of an osmotic tablet with a coating partially composed of a semipermeable membrane with (1A) and without (1B) a pH-responsive layer surrounding the core.

FIG. 2 shows a representative diagram illustrating a cross-sectional view of an osmotic pump tablet with a coating completely composed of a semipermeable membrane with (2A) and without (2B) a pH-responsive layer surrounding the core.

FIG. 3A shows a representative diagram illustrating a cross-sectional view of an osmotic pump tablet with a coating partially composed of both a semipermeable membrane and a pH-responsive layer.

FIG. 3B shows a representative diagram illustrating a cross-sectional view of an osmotic pump tablet with a coating partially composed of a pH-responsive layer.

FIG. 4 shows a representative diagram illustrating how an osmotic pump tablet works.

FIG. 5A shows a representative photograph of a 10 wt % IQP-0528 loaded osmotic pump tablet and an uncoated tablet.

FIG. 5B shows a representative photograph of an osmotic pump tablet.

FIG. 6 shows a representative photograph of a multiswab device fitted with swabs at 11 cm and 5 cm used for collection of vaginal fluid from ewes.

FIG. 7 shows representative data pertaining to the daily (4A) and cumulative (4B) release of the active pharmaceutical ingredient from the osmotic pump tablet.

FIG. 8 shows representative data pertaining to in vitro drug release from an osmotic pump tablet under simulated vaginal conditions.

FIG. 9 shows representative data pertaining to the daily (6A) and cumulative (6B) release of IQP-0528 from an osmotic pump tablet comparing cellulose acetate (CA) and cellulose acetate phthalate (CAP) semipermeable membranes.

FIG. 10 shows representative data pertaining to the dissolution of an osmotic pump tablet at vaginal pH (top) and basic pH (bottom).

FIG. 11 shows representative data pertaining to drug levels in vaginal swabs after the administration of 2 uncoated tablets collected at 11 and 5 cm from the introitus.

FIG. 12 shows representative data pertaining to the vaginal fluid levels of IQP-0528 collected using a multiswab device at two locations, 11 cm and 5 cm.

FIG. 13 shows representative data pertaining to the vaginal distribution of IQP-0528 collected using a multiswab device both proximal (top) and distal (bottom) after insertion of two osmotic tablets.

Additional advantages of the invention will be set forth in part in the description which follows, and in part will be obvious from the description, or can be learned by practice of the invention. The advantages of the invention will be realized and attained by means of the elements and combinations particularly pointed out in the appended claims. It is to be understood that both the foregoing general description and the following detailed description are exemplary and explanatory only and are not restrictive of the invention, as claimed.

### DESCRIPTION

The present invention can be understood more readily by reference to the following detailed description of the invention and the Examples and Figures included herein.

Before the present compounds, compositions, articles, systems, devices, and/or methods are disclosed and described, it is to be understood that they are not limited to specific synthetic methods unless otherwise specified, or to particular reagents unless otherwise specified, as such may, of course, vary. It is also to be understood that the terminology used herein is for the purpose of describing particular aspects only and is not intended to be limiting. Although any methods and materials similar or equivalent to those described herein can be used in the practice or testing of the present invention, example methods and materials are now described.

While aspects of the present invention can be described and claimed in a particular statutory class, such as the system statutory class, this is for convenience only and one of skill in the art will understand that each aspect of the present invention can be described and claimed in any statutory class. Unless otherwise expressly stated, it is in no way intended that any method or aspect set forth herein be construed as requiring that its steps be performed in a specific order. Accordingly, where a method claim does not specifically state in the claims or descriptions that the steps are to be limited to a specific order, it is no way intended that an order be inferred, in any respect. This holds for any possible non-express basis for interpretation, including matters of logic with respect to arrangement of steps or operational flow, plain meaning derived from grammatical organization or punctuation, or the number or type of aspects described in the specification.

Throughout this application, various publications are referenced. The disclosures of these publications in their entireties are hereby incorporated by reference into this application in order to more fully describe the state of the art to which this pertains. The references disclosed are also individually and specifically incorporated by reference herein for the material contained in them that is discussed in the sentence in which the reference is relied upon. Nothing herein is to be construed as an admission that the present invention is not entitled to antedate such publication by virtue of prior invention. Further, the dates of publication provided herein may be different from the actual publication dates, which can require independent confirmation.

### A. DEFINITIONS

As used herein, nomenclature for compounds, including organic compounds, can be given using common names, IUPAC, IUBMB, or CAS recommendations for nomenclature. When one or more stereochemical features are present, Cahn-Ingold-Prelog rules for stereochemistry can be employed to designate stereochemical priority, E/Z specification, and the like. One of skill in the art can readily ascertain the structure of a compound if given a name, either by systemic reduction of the compound structure using naming conventions, or by commercially available software, such as CHEMDRAW™ (Cambridgesoft Corporation, U.S.A.).

As used in the specification and the appended claims, the singular forms “a,” “an” and “the” include plural referents unless the context clearly dictates otherwise. Thus, for example, reference to “a functional group,” “an alkyl,” or “a residue” includes mixtures of two or more such functional groups, alkyls, or residues, and the like.

Ranges can be expressed herein as from “about” one particular value, and/or to “about” another particular value. When such a range is expressed, a further aspect includes from the one particular value and/or to the other particular value. Similarly, when values are expressed as approximations, by use of the antecedent “about,” it will be understood that the particular value forms a further aspect. It will be further understood that the endpoints of each of the ranges are significant both in relation to the other endpoint, and independently of the other endpoint. It is also understood that there are a number of values disclosed herein, and that each value is also herein disclosed as “about” that particular value in addition to the value itself. For example, if the value “10” is disclosed, then “about 10” is also disclosed. It is also understood that each unit between two particular units are also disclosed. For example, if 10 and 15 are disclosed, then 11, 12, 13, and 14 are also disclosed.

References in the specification and concluding claims to parts by weight of a particular element or component in a composition denotes the weight relationship between the element or component and any other elements or components in the composition or article for which a part by weight is expressed. Thus, in a compound containing 2 parts by weight of component X and 5 parts by weight component Y, X and Y are present at a weight ratio of 2:5, and are present in such ratio regardless of whether additional components are contained in the compound.

A weight percent (wt. %) of a component, unless specifically stated to the contrary, is based on the total weight of the formulation or composition in which the component is included.

As used herein, the terms “optional” or “optionally” means that the subsequently described event or circumstance can or cannot occur, and that the description includes instances where said event or circumstance occurs and instances where it does not.

As used herein, the term “subject” can be a vertebrate, such as a mammal, a fish, a bird, a reptile, or an amphibian. Thus, the subject of the herein disclosed methods can be a human, non-human primate, horse, pig, rabbit, dog, sheep, goat, cow, cat, guinea pig or rodent. The term does not denote a particular age or sex. Thus, adult and newborn subjects, as well as fetuses, whether male or female, are intended to be covered. In one aspect, the subject is a mammal A patient refers to a subject afflicted with a disease or disorder. The term “patient” includes human and veterinary subjects.

As used herein, the term “treatment” refers to the medical management of a patient with the intent to cure, ameliorate, stabilize, or prevent a disease, pathological condition, or disorder. This term includes active treatment, that is, treatment directed specifically toward the improvement of a disease, pathological condition, or disorder, and also includes causal treatment, that is, treatment directed toward removal of the cause of the associated disease, pathological condition, or disorder. In addition, this term includes palliative treatment, that is, treatment designed for the relief of symptoms rather than the curing of the disease, pathological condition, or disorder; preventative treatment, that is, treatment directed to minimizing or partially or completely inhibiting the development of the associated disease, pathological condition, or disorder; and supportive treatment, that is, treatment employed to supplement another specific therapy directed toward the improvement of the associated disease, pathological condition, or disorder. In various aspects, the term covers any treatment of a subject, including a mammal (e.g., a human), and includes: (i) preventing the disease from occurring in a subject that can be predisposed to the disease but has not yet been diagnosed as having it; (ii) inhibiting the disease, i.e., arresting its development; or (iii) relieving the disease, i.e., causing regression of the disease. In one aspect, the subject is a mammal such as a primate, and, in a further aspect, the subject is a human. The term “subject” also includes domesticated animals (e.g., cats, dogs, etc.), livestock (e.g., cattle, horses, pigs, sheep, goats, etc.), and laboratory animals (e.g., mouse, rabbit, rat, guinea pig, etc.).

As used herein, the term “prevent” or “preventing” refers to precluding, averting, obviating, forestalling, stopping, or hindering something from happening, especially by advance action. It is understood that where reduce, inhibit or prevent are used herein, unless specifically indicated otherwise, the use of the other two words is also expressly disclosed.

As used herein, the terms “administering” and “administration” refer to any method of providing a pharmaceutical preparation to a subject. Such methods are well known to those skilled in the art and include, but are not limited to, oral administration, transdermal administration, administration by inhalation, nasal administration, topical administration, intravaginal administration, ophthalmic administration, intraaural administration, intracerebral administration, rectal administration, sublingual administration, buccal administration, and parenteral administration, including injectable such as intravenous administration, intra-arterial administration, intramuscular administration, and subcutaneous administration. Administration can be continuous or intermittent. In various aspects, a preparation can be administered therapeutically; that is, administered to treat an existing disease or condition. In further various aspects, a preparation can be administered prophylactically; that is, administered for prevention of a disease or condition.

As used herein, the term “effective amount” refers to an amount that is sufficient to achieve the desired result or to have an effect on an undesired condition. For example, a “therapeutically effective amount” refers to an amount that is sufficient to achieve the desired therapeutic result or to have an effect on undesired symptoms, but is generally insufficient to cause adverse side effects. The specific therapeutically effective dose level for any particular patient will depend upon a variety of factors including the disorder being treated and the severity of the disorder; the specific composition employed; the age, body weight, general health, sex and diet of the patient; the time of administration; the route of administration; the rate of excretion of the specific compound employed; the duration of the treatment; drugs used in combination or coincidental with the specific compound employed and like factors well known in the medical arts. For example, it is well within the skill of the art to start doses of a compound at levels lower than those required to achieve the desired therapeutic effect and to gradually increase the dosage until the desired effect is achieved. If desired, the effective daily dose can be divided into multiple doses for purposes of administration. Consequently, single dose compositions can contain such amounts or submultiples thereof to make up the daily dose. The dosage can be adjusted by the individual physician in the event of any contraindications. Dosage can vary, and can be administered in one or more dose administrations daily, for one or several days. Guidance can be found in the literature for appropriate dosages for given classes of pharmaceutical products. In further various aspects, a preparation can be administered in a “prophylactically effective amount”; that is, an amount effective for prevention of a disease or condition.

The term “pharmaceutically acceptable” describes a material that is not biologically or otherwise undesirable, i.e., without causing an unacceptable level of undesirable biological effects or interacting in a deleterious manner.

As used herein, the term “derivative” refers to a compound having a structure derived from the structure of a parent compound (e.g., a compound disclosed herein) and whose structure is sufficiently similar to those disclosed herein and based upon that similarity, would be expected by one skilled in the art to exhibit the same or similar activities and utilities as the claimed compounds, or to induce, as a precursor, the same or similar activities and utilities as the claimed compounds. Exemplary derivatives include salts, esters, amides, salts of esters or amides, and N-oxides of a parent compound.

As used herein, the term “pharmaceutically acceptable carrier” refers to sterile aqueous or nonaqueous solutions, dispersions, suspensions or emulsions, as well as sterile powders for reconstitution into sterile injectable solutions or dispersions just prior to use. Examples of suitable aqueous and nonaqueous carriers, diluents, solvents or vehicles include water, ethanol, polyols (such as glycerol, propylene glycol, polyethylene glycol and the like), carboxymethylcellulose and suitable mixtures thereof, vegetable oils (such as olive oil) and injectable organic esters such as ethyl oleate. Proper fluidity can be maintained, for example, by the use of coating materials such as lecithin, by the maintenance of the required particle size in the case of dispersions and by the use of surfactants. These compositions can also contain adjuvants such as preservatives, wetting agents, emulsifying agents and dispersing agents. Prevention of the action of microorganisms can be ensured by the inclusion of various antibacterial and antifungal agents such as paraben, chlorobutanol, phenol, sorbic acid and the like.

The term “stable,” as used herein, refers to compounds that are not substantially altered when subjected to conditions to allow for their production, detection, and, in certain aspects, their recovery, purification, and use for one or more of the purposes disclosed herein.

The terms “controlled-release device” and “osmotic device” are generally used herein interchangeably. Basically, an osmotic device is a controlled-release device that comprises a semipermeable membrane surrounding the polymer-containing core, and optionally one or more other coatings and/or membranes. The preformed passageway is disposed at least through the semipermeable membrane. The core can be a unitary core and/or a single core, a bi-layered core, or a multi-layered core. The layers in the core can be stacked, substantially concentric, or substantially eccentric arrangement. The core of the osmotic device can be a bi-layered core wherein the nucleus of the core is an inert composition containing swellable agents and the layer surrounding the nucleus is a polymer-containing layer. The osmotic device can also comprise an inert water soluble or erodible coat composition surrounding the semipermeable membrane. The preformed passageway can be disposed through the inert water soluble or erodible coat composition and the semipermeable membrane. The semipermeable membrane can contain porogens to alter the rate of water entry into the core.

The term “core,” as used herein, refers to the body of an osmotic device that comprises a water-swellable gel-forming polymer(s) and/or osmotic agent(s) and, optionally, a pharmacologically active agent. In certain aspects, the core is divided into two or more layers or laminas. In certain aspects, the core is not divided into two or more layers or laminas. The core is considered to be the composition enclosed within the wall, e.g., semipermeable membrane, of the osmotic device. The ingredients of the core may be present as a heterogeneous mixture or homogeneous mixture. A homogeneous mixture is one wherein all of the ingredients have been thoroughly mixed such that the composition of the formulation is substantially the same throughout different portions of the core. The combined step of mixing and directly compressing the ingredients of the core generally provides a homogeneous mixture. A heterogeneous mixture is one wherein the ingredients of the core are divided into two or more groups that are processed separately to form two or more respective blends, at least one of which contains the water-swellable gel-forming polymer and at least one of which contains the pharmacologically active agent. The blends are then mixed together and compressed to form the unitary core. A heterogeneous mixture can be obtained by wet granulation, dry granulation, pelleting, or combinations thereof.

The term “osmotic pressure,” as used herein, refers to the hydrostatic pressure produced by a differential in the concentrations of solutes between the core and the external environment of use of the osmotic device.

The term “osmotic agent,” as used herein, refers to any non-swellable, low molecular weight polymeric or non-polymeric material that is soluble (i.e., partially or totally solubilized) and which exhibits an osmotic pressure gradient across the semipermeable membrane, thus increasing the hydrostatic pressure inside the core of the controlled release device. Examples of osmotic agents include, but are not limited to, magnesium sulfate, magnesium chloride, calcium chloride, sodium chloride, lithium chloride, potassium sulfate, sodium carbonate, sodium sulfite, lithium hydrogen phosphate, lithium sulfate, potassium chloride, sodium hydrogen phosphate, potassium hydrogen phosphate, lithium dihydrogen phosphate, sodium dihydrogen phosphate, potassium dihydrogen phosphate, sodium sulfate, sodium alginate, gelatine, sodium starch glycolate, mannitol, xylitol, urea, urea derivatives, sorbitol, inositol, raffinose, sucrose, glycine, leucine, alanine, methionine, glucose, fructose, lactose, inulin, instant sugar, citric acid, succinic acid, tartaric acid, mannitol, xylitol, sodium acetate, magnesium stearate, sodium benzoate, sodium citrate, sodium ascorbate, and carbohydrates, and mixtures thereof.

The term “water-swellable polymer,” as used herein, refers to a high molecular weight polymer which is substantially water-soluble, water-swellable, and preferably water-gelling, forming a hydrogel. Water-swellable polymers are typically crosslinked polymers. Examples of polymers useful in the present invention include hydroxypropyl cellulose, hydroxyethyl cellulose, hydroxy propyl methyl cellulose, carboxy methyl cellulose, carrageenan and polysaccharide gums, polyacrylic acid, and polymethacrylic acid.

The term “substantially,” as used herein, means that the subsequently described event or circumstance completely occurs or that the subsequently described event or circumstance generally, typically, or approximately occurs. For example, when the specification discloses that method steps are performed substantially simultaneously, a person skilled in the relevant art would readily understand that the steps need not be synchronized. Rather, this term conveys to a person skilled in the relevant art that the method steps can be synchronized, can be overlapping in time, or can be separated by a technically insignificant (e.g., commercially insignificant) amount of time.

The term “inelastic,” as used herein, refers to materials that do not stretch by 50% or more and to materials that stretch by that amount but do not retract by more than 30%. Inelastic materials also include materials that do not extend, e.g., which tear, when exposed to a stretching force.

The term “water-insoluble,” as used herein, refers to materials that are not susceptible to being dissolved in water. Specific examples of water-insoluble organosoluble polymers are cellulose ether, cellulose ester, or cellulose ether-ester (e.g., ethyl cellulose, acetyl cellulose, and nitrocellulose). Other water insoluble organosoluble polymers that can be used include acrylic and/or methacrylic ester polymers, polymers or copolymers of acrylate of methacrylate polyvinyl esters, polyvinyl acetates, polyarylic acid esters, and butadiene styrene copolymers, and the like.

The definition of the term “semipermeable membrane” is known to a person skilled in the relevant art. By semipermeable membrane is meant a membrane that permits the influx of a liquid from the exterior of the device to the interior of the device, while at the same time allowing release of the active agent in the core by osmotic pumping through the preformed passageway in the semipermeable membrane.

The term “macromolecule,” as used herein, refers to molecules having a molecular weight of more than 2,000. Examples of macromolecules include but are not limited to synthetic polymers, proteins, polysaccharides, and certain peptides.

The term “hydrophilic small molecule,” as used herein, refers to a molecule with a molecular weight of 2,000 or below and having a water solubility of about 0.1 mg/mL or greater. Hydrophilic small molecules also include smaller peptides and many drugs.

Certain materials, compounds, compositions, and components disclosed herein can be obtained commercially or readily synthesized using techniques generally known to those of skill in the art. For example, the starting materials and reagents used in preparing the disclosed compounds and compositions are either available from commercial suppliers such as Aldrich Chemical Co., (Milwaukee, Wis.), Acros Organics (Morris Plains, N.J.), Fisher Scientific (Pittsburgh, Pa.), or Sigma (St. Louis, Mo.) or are prepared by methods known to those skilled in the art following procedures set forth in references such as Fieser and Fieser's Reagents for Organic Synthesis, Volumes 1-17 (John Wiley and Sons, 1991); Rodd's Chemistry of Carbon Compounds, Volumes 1-5 and Supplementals (Elsevier Science Publishers, 1989); Organic Reactions, Volumes 1-40 (John Wiley and Sons, 1991); March's Advanced Organic Chemistry, (John Wiley and Sons, 4th Edition); and Larock's Comprehensive Organic Transformations (VCH Publishers Inc., 1989).

Unless otherwise expressly stated, it is in no way intended that any method set forth herein be construed as requiring that its steps be performed in a specific order. Accordingly, where a method claim does not actually recite an order to be followed by its steps or it is not otherwise specifically stated in the claims or descriptions that the steps are to be limited to a specific order, it is no way intended that an order be inferred, in any respect. This holds for any possible non-express basis for interpretation, including: matters of logic with respect to arrangement of steps or operational flow; plain meaning derived from grammatical organization or punctuation; and the number or type of embodiments described in the specification.

Disclosed are the components to be used to prepare the compositions of the invention as well as the compositions themselves to be used within the methods disclosed herein. These and other materials are disclosed herein, and it is understood that when combinations, subsets, interactions, groups, etc. of these materials are disclosed that while specific reference of each various individual and collective combinations and permutation of these compounds cannot be explicitly disclosed, each is specifically contemplated and described herein. For example, if a particular compound is disclosed and discussed and a number of modifications that can be made to a number of molecules including the compounds are discussed, specifically contemplated is each and every combination and permutation of the compound and the modifications that are possible unless specifically indicated to the contrary. Thus, if a class of molecules A, B, and C are disclosed as well as a class of molecules D, E, and F and an example of a combination molecule, A-D is disclosed, then even if each is not individually recited each is individually and collectively contemplated meaning combinations, A-E, A-F, B-D, B-E, B-F, C-D, C-E, and C-F are considered disclosed. Likewise, any subset or combination of these is also disclosed. Thus, for example, the sub-group of A-E, B-F, and C-E would be considered disclosed. This concept applies to all aspects of this application including, but not limited to, steps in methods of making and using the compositions of the invention. Thus, if there are a variety of additional steps that can be performed it is understood that each of these additional steps can be performed with any specific embodiment or combination of embodiments of the methods of the invention.

It is understood that the compositions disclosed herein have certain functions. Disclosed herein are certain structural requirements for performing the disclosed functions and it is understood that there are a variety of structures that can perform the same function that are related to the disclosed structures, and that these structures will typically achieve the same result.

### B. CONTROLLED-RELEASE DEVICES

In one aspect, the invention relates to a controlled-release device comprising: a) a core comprising: i) a water-swellable gel-forming polymer; and ii) optionally, an osmotic agent; and iii) optionally, a pharmacologically active agent; b) a substantially inelastic, water-insoluble coating substantially enclosing the core, wherein at least a portion of the coating comprises a semipermeable membrane; and c) at least one orifice in the coating, wherein the orifice is positioned and dimensioned to allow controlled-release of the polymer from the core in response to swelling of the polymer, wherein the core comprises no more than 15 wt % of the osmotic agent. In a further aspect, the core comprises no more than 5 wt % of the osmotic agent.

In one aspect, the invention relates to a controlled-release device comprising: a) a core comprising: i) a water-swellable gel-forming polymer and/or an osmotic agent; and ii) optionally, a pharmacologically active agent; and b) a water-insoluble coating substantially enclosing the core, wherein at least a portion of the coating comprises a pH-responsive material.

Controlled-release devices of the present invention can include, for example, particles, filaments, implants, tablets, osmotic pumps, medical devices, and the like. In various aspects, the controlled-release devices of the present invention respond rapidly to changes such as dilution effects in the external environment, e.g., by body fluid flanges, whereas sustained release articles not (see, i.e., Cowsar in “Advances in Experimental Medicine and Biology”, Vol. 49, “Controlled-release of Biologically Active Agents,” Ed. Tanquary and Lacey, Plenum Press, 1974).

The controlled-release device of the present invention is capable of providing sustained delivery of a gel-forming polymer in a substantially zero order release profile. By substantially zero order it is meant that a substantially constant amount of polymer is released over a given period of time. In various aspects, the release rate of the polymer from the device may be modified by changing the nature and concentration of the polymer. In a further aspect, changing device geometry such as surface area and semi-permeable membrane thickness can be used to modify the release rate of the polymer. In a still further aspect, the core loading may not affect the release rate, but will instead affect the release duration.

In various aspects, the controlled-release device may encompass a variety of shapes and sizes, provided the device is compatible with mucosal tissue membrane administration to the subject and with the requirements imposed by drug delivery kinetics. Thus, the controlled-release device may be standard, plain, convex, bevel, capsule, bullet, square, rectangle, pillow, pillow, diamond, hexagon, pentagon, triangle or oval shaped or of any other shape known in the art.

In a further aspect, the diameter of the controlled-release device may range from about 2 mm to about 20 mm. In a still further aspect, the diameter of the controlled-release device may range from about 2 mm to about 15 mm. In yet a further aspect, the diameter of the controlled-release device may range from about 2 mm to about 10 mm. In an even further aspect, the diameter of the controlled-release device may range from about 2 mm to about 5 mm. In a still further aspect, the diameter of the controlled-release device may range from about 5 mm to about 20 mm. In yet a further aspect, the diameter of the controlled-release device may range from about 10 mm to about 20 mm. In an even further aspect, the diameter of the controlled-release device may range from about 15 mm to about 20 mm.

In various aspects, the controlled-release device is an osmotic pump tablet. Such tablets may be made, for example, by providing a core comprising a water-swellable gel-forming polymer, optionally, an osmotic agent, and, optionally, a pharmacologically active agent; substantially enclosing the core within a substantially inelastic, water-insoluble coating, wherein at least a portion of the coating comprises a semipermeable membrane; and creating at least one orifice in the coating, wherein the core comprises no more than 15 wt % of the osmotic agent.

In various aspects, the osmotic pump tablet may be mounted in another device like an intravaginal ring, a pessary, or a tampon-shaped device.

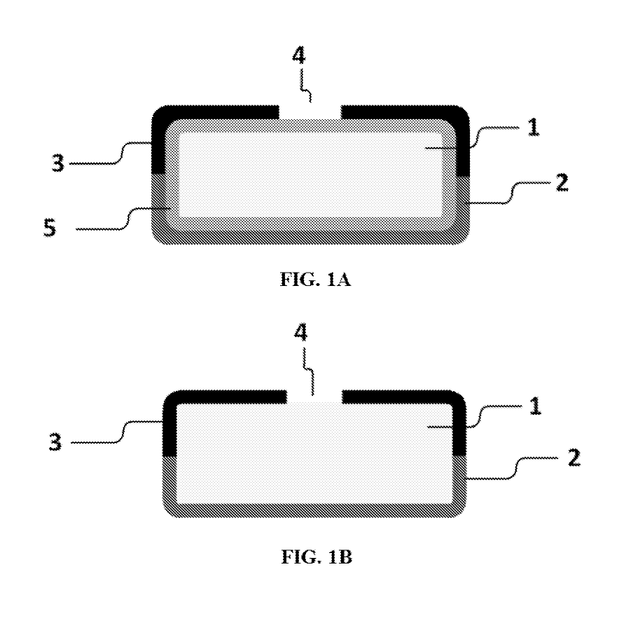

In one aspect the controlled-release device of the present invention delivers a water-swellable gel-forming polymer and/or a pharmacologically active agent to a surrounding environment as follows. Referring to FIG. 1A, the controlled-release device comprises a core (1) containing a water-swellable gel-forming polymer, a pharmacologically active agent, and optionally, an osmotic agent. The core is surrounded be a pH-responsive layer (5). Upon a change in pH, the pH-responsive layer (5) partially or fully dissolves, allowing fluid to enter the core (1) through the semipermeable membrane (2). The polymer will dissolve and/or swell in response to the fluid thereby creating an osmotic pressure gradient across the semipermeable membrane (3). This gradient provides the force required to push the polymer and/or active agent through the orifice (4) until osmotic equilibrium between the core and the environment of use is reached. This equilibrium of osmotic forces occurs gradually over a period of time thereby serving to control the release of, and thus the release profile, for the polymer and/or active agent. The release of the active agent slows as osmotic equilibrium is approached, and then stops when osmotic equilibrium is reached. The extent to which the release of the polymer and/or active agent is controlled is known to depend upon a number of other variables including, for example, the permeability of the semipermeable membrane (3) and the magnitude of the osmotic pressure gradient.

In one aspect the controlled-release device of the present invention delivers a water-swellable gel-forming polymer and/or a pharmacologically active agent to a surrounding environment as follows. Referring to FIG. 1B, the controlled-release device comprises a core (1) containing a water-swellable gel-forming polymer, a pharmacologically active agent, and optionally, an osmotic agent. The core is surrounded by a water-insoluble coating (2), wherein a portion of the coating comprises semipermeable membrane (3), having an orifice (4) that delivers the polymer and/or active agent to a surrounding environment in a controlled manner. The polymer will dissolve and/or swell in the fluid that enters into the core (1) through the semipermeable membrane (3) thereby creating an osmotic pressure gradient across the semipermeable membrane (3). This gradient provides the force required to push the polymer and/or active agent through the orifice (4) until osmotic equilibrium between the core and the environment of use is reached.

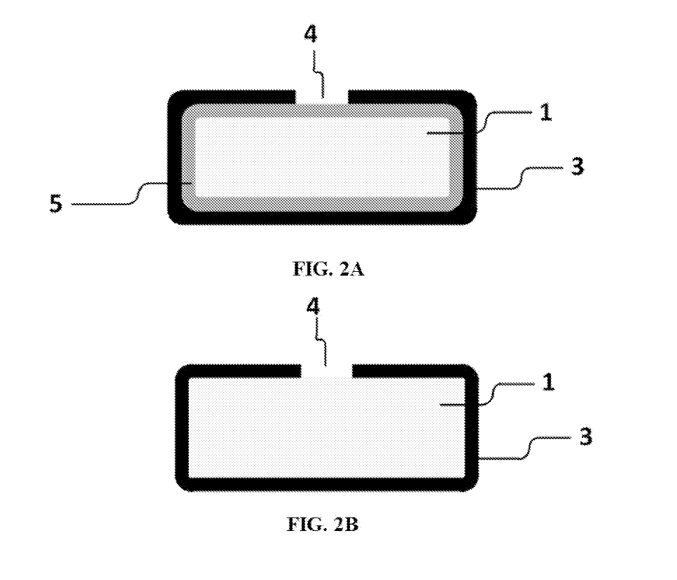

In one aspect the controlled-release device of the present invention delivers a water-swellable gel-forming polymer and/or a pharmacologically active agent to a surrounding environment as follows. Referring to FIG. 2A, the controlled-release device comprises a core (1) containing a water-swellable gel-forming polymer, a pharmacologically active agent, and optionally, an osmotic agent. The core is surrounded be a pH-responsive layer (5). Upon a change in pH, the pH-responsive layer (5) partially or fully dissolves, allowing fluid to enter the core (1) through the semipermeable membrane (2). The polymer will dissolve and/or swell in response to the fluid thereby creating an osmotic pressure gradient across the semipermeable membrane (3). This gradient provides the force required to push the polymer and/or active agent through the orifice (4) until osmotic equilibrium between the core and the environment of use is reached.

In one aspect the controlled-release device of the present invention delivers a water-swellable gel-forming polymer and/or a pharmacologically active agent to a surrounding environment as follows. Referring to FIG. 2B, the controlled-release device comprises a core (1) containing a water-swellable gel-forming polymer, a pharmacologically active agent, and optionally, an osmotic agent. The core is surrounded by a water-insoluble coating, wherein the coating essentially comprises a semipermeable membrane (3), having an orifice (4) that delivers the polymer and/or active agent to a surrounding environment in a controlled manner. The polymer will dissolve and/or swell in the fluid that enters into the core (1) through the semipermeable membrane (3) thereby creating an osmotic pressure gradient across the semipermeable membrane (3). This gradient provides the force required to push the polymer and/or active agent through the orifice (4) until osmotic equilibrium between the core and the environment of use is reached.

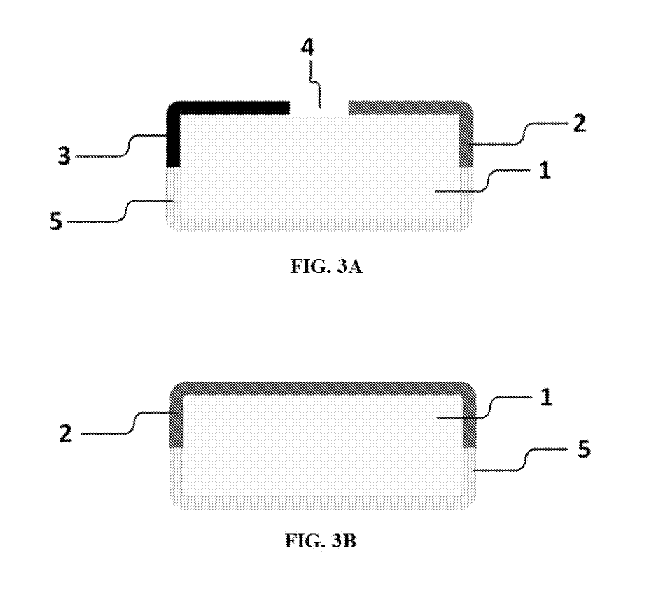

In one aspect the controlled-release device of the present invention delivers a water-swellable gel-forming polymer and/or a pharmacologically active agent to a surrounding environment as follows. Referring to FIG. 3A, the controlled-release device comprises a core (1) containing a water-swellable gel-forming polymer, a pharmacologically active agent, and optionally, an osmotic agent. The core is surrounded by a water-insoluble coating (2), wherein the coating comprises a semipermeable membrane (3) and a pH-responsive material (5), having an orifice (4) that delivers the polymer and/or active agent to a surrounding environment in a controlled manner. Upon a change in pH, the pH-responsive layer (5) partially or fully dissolves, allowing fluid to enter the core (1) through the semipermeable membrane (2). Additionally, fluid may also enter through into the core (1) through the semipermeable membrane (3). The polymer will dissolve and/or swell in response to the fluid thereby creating an osmotic pressure gradient across the semipermeable membrane (3). This gradient provides the force required to push the polymer and/or active agent through the orifice (4) until osmotic equilibrium between the core and the environment of use is reached.

In one aspect the controlled-release device of the present invention delivers a water-swellable gel-forming polymer and/or a pharmacologically active agent to a surrounding environment as follows. Referring to FIG. 3B, the controlled-release device comprises a core (1) containing a water-swellable gel-forming polymer, a pharmacologically active agent, and optionally, an osmotic agent. The core is surrounded by a water-insoluble coating, wherein the coating comprises a pH-responsive material (5). Upon a change in pH, the pH-responsive layer (5) partially or fully dissolves, allowing fluid to enter the core (1) through the pH-responsive material (5). The polymer will dissolve and/or swell in response to the fluid thereby creating an osmotic pressure gradient across the semipermeable membrane (3). This gradient provides the force required to push the polymer and/or active agent through the pH-responsive material (5) until osmotic equilibrium between the core and the environment of use is reached.

In various aspects of the present invention, the controlled-release device is formulated to release the water-swellable gel-forming polymer and/or pharmacologically active agent in vivo and/or in the presence of a change in pH for at least 10 minutes. In a further aspect, release of the polymer and/or active agent continues for at least 30 minutes. In a still further aspect, release of the polymer and/or active agent continues for at least 1 hour. In yet a further aspect, release of the polymer and/or active agent continues for at least 5 hours. In an even further aspect, release of the polymer and/or active agent continues for at least 10 hours. In a still further aspect, release of the polymer and/or active agent continues for at least 12 hours. In yet a further aspect, release of the polymer and/or active agent continues for at least 24 hours. In an even further aspect, release of the polymer and/or active agent continues for at least 2 days. In a still further aspect, release of the polymer and/or active agent continues for at least 3 days. In yet a further aspect, release of the polymer and/or active agent continues for at least 4 days. In an even further aspect, release of the polymer and/or active agent continues for at least 5 days. In a still further aspect, release of the polymer and/or active agent continues for at least 6 days. In yet a further aspect, release of the polymer and/or active agent continues for at least 7 days.

In various aspects, the controlled-release device exhibits release rates of from about 0.001 mg of polymer and/or pharmacologically active agent per day to about 10 mg of polymer and/or pharmacologically active agent per day. In a further aspect, controlled-release device exhibits release rates of from about 0.001 mg to about 10 mg, from about 0.005 mg to about 10 mg, from about 0.01 mg to about 10 mg, from about 0.025 mg to about 10, from about 0.05 mg to about 10 mg, from about 0.075 mg to about 10 mg, from about 0.1 mg to about 10 mg, from about 0.15 mg to about 10 mg, from about 0.2 mg to about 10 mg, from about 0.5 mg to about 10 mg, from about 0.75 mg to about 10 mg, from about 1 mg to about 10 mg, from about 2 mg to about 10, from about 3 mg to about 10, from about 4 mg to about 10 mg, from about 5 mg to about 10 mg, from about 0.001 mg to about 5 mg, from about 0.001 mg to about 4 mg, from about 0.001 mg to about 3 mg, from about 0.001 mg to about 2 mg, from about 0.001 mg to about 1 mg, from about 0.001 mg to about 0.75 mg, from about 0.001 mg, to about 0.5 mg, from about 0.001 mg to about 0.2 mg, from about 0.001 mg to about 0.015 mg, from about 0.001 mg to about 10 mg, from about 0.001 mg to about 0.075 mg, from about 0.001 mg to about 0.05 mg, from about 0.001 mg to about 0.025 mg, from about 0.001 mg to about 0.01 mg, or from about 0.001 mg to about 0.005 mg.

In various aspects, the controlled-release device of the present invention further comprises a pH-responsive material covering the core. In a further comprising a pH-responsive material covering the membrane and/or the orifice. In a still further aspect, the pH-responsive material is water-insoluble at a pH of less than about 6. In yet a further aspect, the pH-responsive material is water-insoluble at a pH of less than about 5. In an even further aspect, the pH-responsive material is water-insoluble at a pH of less than about 4. In a still further aspect, the pH-responsive material is water-soluble at a pH of greater than about 6. In yet a further aspect, the pH-responsive material is water-soluble at a pH of greater than about 7. In an even further aspect, the pH-responsive material is water-soluble at a pH of greater than about 8.

In a further aspect, the controlled-release device of the present invention further comprises a pH-responsive material covering the core, wherein the pH-responsive material is water-insoluble at a pH of less than about 5, wherein the pH-responsive material is water-soluble at a pH of greater than about 7, and wherein the membrane forms substantially all of the coating.

In one aspect, the invention relates to a controlled-release device comprising: a) a core comprising: i) a water-swellable gel-forming polymer and/or an osmotic agent; and ii) at least one lubricant; b) a substantially inelastic, water-insoluble coating substantially enclosing the core, wherein at least a portion of the coating comprises a semipermeable membrane; and c) at least one orifice in the coating, wherein the orifice is positioned and dimensioned to allow controlled-release of the lubricant from the core. In a further aspect, the core comprises no more than 15 wt % of the osmotic agent. In a still further aspect, the core comprises no more than 5 wt % of the osmotic agent. In an even further aspect, the device further comprises a pH-responsive material covering the core.

In a further aspect, the lubricant is water-soluble. In a still further aspect, the lubricant is not water-soluble. In yet a further aspect, the lubricant comprises a lubricating agent. In an even further aspect, the lubricant is selected from pyridine, squalene, urea, complex alcohols, aldehydes, ketones, stearic acid, stearate, isopropyl palmitate, petrolatum, aloe barbadensis (Aloe Vera) leaf juice, cucumus sativus extract, helianthus annulus seed oil, soybean sterol, vitamin E acetate, vitamin A palmitate, provitamin B5, sodium acrylate/acryloyldimethyl taurate copolymer, dimethicone, dimethiconol, silicone, silicone oil, glyceryl stearate, ceylalcohol, lecithin, mineral oil, sodium PCA, potassium lactate, collagen, aminoacids, triethanolamine, DMDM, hydantoin, iodopropynyl, butylcarbamate, disodium EDTA, and titanium dioxide.

In one aspect, the invention relates to a controlled-release device comprising: a) a core comprising: i) a water-swellable gel-forming polymer and/or an osmotic agent; and ii) at least one biologic; b) a substantially inelastic, water-insoluble coating substantially enclosing the core, wherein at least a portion of the coating comprises a semipermeable membrane; and c) at least one orifice in the coating, wherein the orifice is positioned and dimensioned to allow controlled-release of the biologic from the core. In a further aspect, the core comprises no more than 15 wt % of the osmotic agent. In a still further aspect, the core comprises no more than 5 wt % of the osmotic agent. In yet a further aspect, the device further comprises a pH-responsive material covering the core.

In a further aspect, the biologic is a probiotic. In a still further aspect, the probiotic is selected from selected from Lactobacillus bulgaricus, Lactobacillus casei subsp. Rhamnosus, Lactobacillus casei subsp. Casei, Lactobacillus salivarius, Lactobacillus brevis, Lactobacillus reuteri, Lactococcus lactis subsp. Lactis, Enterococcus faecium, Lactobacillus plantarum, Streptococcus thermophilus, Bifidobacterium infantis, Bifidobacterium bifidum, Bifidobacterium longum, Saccharomyces boulardii, and Lactobacillus acidophilus, Lactobacillus crispatus, whey proteins, lysozyme, lactoferrin, lactoperoxidase, xanthine oxidase, vitamin-binding proteins and immunoglobulins, mannose, oligosaccharides, starches, mannan oligosaccharides, trans-galacto-oligosaccharide, inulin, and fructo-oligosaccharide.

1. Core

In one aspect, the controlled-release device of the present invention comprises a core comprising: a) a water-swellable gel-forming polymer; b) optionally, an osmotic agent; and c) optionally, a pharmacologically active agent, wherein the core comprises no more than 15 wt % of the osmotic agent. The core material may be substantially liquid, or may contain a substantial amount of air.

In one aspect, the controlled-release device of the present invention comprises a core comprising: a) water-swellable gel-forming polymer and/or an osmotic agent; and b) at least one lubricant. In a further aspect, the core comprises no more than 15 wt % of the osmotic agent. In a still further aspect, the core comprises no more than 5 wt % of the osmotic agent.

In one aspect, the controlled-release device of the present invention comprises a core comprising: a) water-swellable gel-forming polymer and/or an osmotic agent; and b) at least one biologic. In a further aspect, the core comprises no more than 15 wt % of the osmotic agent. In a still further aspect, the core comprises no more than 5 wt % of the osmotic agent.

In various aspects, the core is substantially enclosed within a coating comprising at least one orifice.

In various aspects, changing the device geometry (i.e., core excipient) can be used to modify the polymer release rate. In various aspects, the core loading may not affect the release rate, but will instead affect the release duration. In various aspects, the devices exhibit release rates of from about 0.001 mg to about 10 mg. In a further aspect, the devices exhibit release rates of from about 0.005 mg to about 10 mg. In a still further aspect, the devices exhibit release rates of from about 0.010 mg to about 10 mg. In yet a further aspect, the devices exhibit release rates of from about 0.025 mg to about 10 mg. In an even further aspect, the devices exhibit release rates of from about 0.050 mg to about 10 mg. In a still further aspect, the devices exhibit release rates of from about 0.075 mg to about 10 mg. In yet a further aspect, the devices exhibit release rates of from about 0.10 mg to about 10 mg. In an even further aspect, the devices exhibit release rates of from about 0.15 mg to about 10 mg. In a still further aspect, the devices exhibit release rates of from about 0.20 mg to about 10 mg. In yet a further aspect, the devices exhibit release rates of from about 0.50 mg to about 10 mg. In an even further aspect, the devices exhibit release rates of from about 0.75 mg to about 10 mg. In a still further aspect, the devices exhibit release rates of from about 1 mg to about 10 mg. In yet a further aspect, the devices exhibit release rates of from about 2 mg to about 10 mg. In an even further aspect, the devices exhibit release rates of from about 3 mg to about 10 mg. In a still further aspect, the devices exhibit release rates of from about 4 mg to about 10 mg. In yet a further aspect, the devices exhibit release rates of from about 5 mg to about 10 mg. In an even further aspect, the devices exhibit release rates of from about 0.001 mg to about 5 mg. In a still further aspect, the devices exhibit release rates of from about 0.001 mg to about 4 mg. In yet a further aspect, the devices exhibit release rates of from about 0.001 mg to about 3 mg. In an even further aspect, the devices exhibit release rates of from about 0.001 mg to about 2 mg. In a still further aspect, the devices exhibit release rates of from about 0.001 mg to about 1 mg. In yet a further aspect, the devices exhibit release rates of from about 0.001 mg to about 0.75 mg. In an even further aspect, the devices exhibit release rates of from about 0.001 mg to about 0.50 mg. In a still further aspect, the devices exhibit release rates of from about 0.001 mg to about 0.20 mg. In yet a further aspect, the devices exhibit release rates of from about 0.001 mg to about 0.15 mg. In an even further aspect, the devices exhibit release rates of from about 0.001 mg to about 0.10 mg. In a still further aspect, the devices exhibit release rates of from about 0.001 mg to about 0.075 mg. In yet a further aspect, the devices exhibit release rates of from about 0.001 mg to about 0.05 mg. In an even further aspect, the devices exhibit release rates of from about 0.001 mg to about 0.025 mg. In a still further aspect, the devices exhibit release rates of from about 0.001 mg to about 0.010 mg. In yet a further aspect, the devices exhibit release rates of from about 0.001 mg to about 0.005 mg.

In a further aspect, the core comprises no more than 15 wt % of the osmotic agent. In a still further aspect, the core comprises no more than 12 wt % of the osmotic agent. In yet a further aspect, the core comprises no more than 10 wt % of the osmotic agent. In an even further aspect, the core comprises no more than 8 wt % of the osmotic agent. In a still further aspect, the core comprises no more than 6 wt % of the osmotic agent. In yet a further aspect, the core comprises no more than 5 wt % of the osmotic agent. In an even further aspect, the core comprises no more than 4 wt % of the osmotic agent. In an even further aspect, the core comprises no more than 3 wt % of the osmotic agent. In a still further aspect, the core comprises no more than 2 wt % of the osmotic agent. In yet a further aspect, the core comprises no more than 1 wt % of the osmotic agent. In an even further aspect, the core does not comprise an osmotic agent.

a. Gel-Forming Polymer

In one aspect, the core of the disclosed device comprises a water-swellable gel-forming polymer. Water-swellable polymers are specifically high molecular weight hydrophilic gelling polymers that imbibe water, forming a gel that exerts internal pressure, and is eventually forced out of the drug releasing orifice. This gel assists in retention of the controlled-release device in the mucous membrane tissue.

Examples of water-swellable polymers include, but are not limited to, carrageenan, cellulose ethers, hydroxy ethyl cellulose, hydroxy propyl cellulose, hypromellose, carboxy methyl cellulose, polysaccharide gums, guar gum, xanthan gums, pectins, polyacrylic acid, polyvinyl pyrrolidinone, polyvinyl alcohol, or hydroxyl ethylcellulose with molecular weights greater than 800,000. Thus, in various aspects, the polymer is a high molecular weight polymer. In a further aspect, the polymer has an average molecular weight of at least about 800,000. In a still further aspect, the polymer has an average molecular weight of at least about 900,000. In yet a further aspect, the polymer has an average molecular weight of at least about 1,000,000. In an even further aspect, the polymer has an average molecular weight of at least about 1,500,000. In a still further aspect, the polymer has an average molecular weight of at least about 2,000,000. In yet a further aspect, the polymer has an average molecular weight of at least about 3,000,000. In an even further aspect, the polymer has an average molecular weight of at least about 4,000,000. In a still further aspect, the polymer has an average molecular weight of at least about 5,000,000. In yet a further aspect, the polymer has an average molecular weight of at least about 6,000,000.

Water-swellable polymers of the present invention may be pH responsive polymers. Thus, in various aspects, the polymer is water-insoluble at a pH of less than about 6. In a still further aspect, the polymer is water-insoluble at a pH of less than about 5. In yet a further aspect, the polymer is water-insoluble at a pH of less than about 4. In an even further aspect, the polymer is water-soluble at a pH of greater than about 6. In a still further aspect, the polymer is water-soluble at a pH of greater than about 7. In yet a further aspect, the polymer is water-soluble at a pH of greater than about 8.

The rate of release of a substance from the present devices may be controlled by controlling the rate of swelling of the water-swellable polymer. One way to control release rates is therefore through the selection of water-swellable polymers having different rates of swelling. Thus, for example, lambda carrageenan, iota carrageenan, kappa carrageenan, or a mixture of any two or more thereof may be used as the water-swellable polymer to adjust release rates.

In various aspects, the polymer of the present invention is a cellulose ether. In a further aspect, the polymer is selected from hydroxy propyl cellulose, hydroxy ethyl cellulose and hydroxy propyl methyl cellulose of varying molecular weights, polyethylene glycols and polyethylene glycol ethers of varying molecular weights. In a still further aspect, the polymer is selected from hydroxy propyl cellulose and hydroxy ethyl cellulose. In yet a further aspect, the polymer is hydroxyl propyl cellulose. In an even further aspect, the polymer is hydroxy ethyl cellulose.

In various aspects, the polymer of the present invention is a natural gelling polymer. In a further aspect, the natural gelling polymer is selected from carrageenan, xanthan gum, and guar gum.

In various aspects, the polymer of the present invention may be blended with a salt of sodium or potassium. In a further aspect, the salt of sodium or potassium is selected from sodium and/or potassium chloride, -acetate, -citrate, -lactate, and -tartarate.

In various aspects, the polymer is blended with a second high molecular weight polymer. In a further aspect, the polymer is blended with two or more high molecular weight polymers.

In a further aspect, the polymer is a crosslinked polymer. In a still further aspect, the polymer is a boronic acid-hydroxamic acid crosslinked polymer. In yet a further aspect, the polymer is a boronic acid-diol crosslinked polymer. Exemplary diols include, but are not limited to dextran, poly vinyl alcohol, and carnohydrate containing polymers.

In a further aspect, the polymer is present in an amount from about 5 wt % to about 99 wt % of the core. In a still further aspect, the polymer is present in an amount from about 5 wt % to about 90 wt % of the core. In yet a further aspect, the polymer is present in an amount from about 5 wt % to about 75 wt % of the core. In an even further aspect, the polymer is present in an amount from about 5 wt % to about 50 wt % of the core. In a still further aspect, the polymer is present in an amount from about 5 wt % to about 25 wt % of the core. In yet a further aspect, the polymer is present in an amount from about 5 wt % to about 15 wt % of the core. In an even further aspect, the polymer is present in an amount from about 5 wt % to about 10 wt % of the core. In a still further aspect, the polymer is present in an amount from about 10 wt % to about 99 wt % of the core. In yet a further aspect, the polymer is present in an amount from about 15 wt % to about 99 wt % of the core. In an even further aspect, the polymer is present in an amount from about 25 wt % to about 99 wt % of the core. In a still further aspect, the polymer is present in an amount from about 50 wt % to about 99 wt % of the core. In yet a further aspect, the polymer is present in an amount from about 75 wt % to about 99 wt % of the core. In an even further aspect, the polymer is present in an amount from about 90 wt % to about 99 wt % of the core.

In a further aspect, the polymer is a pharmacologically active agent.

In a further aspect, the polymer is a pharmacologically active agent and present in an effective amount. In a still further aspect, the effective amount is from about 0.01 wt % to about 50 wt %. In yet a further aspect, the effective amount is from about 0.01 wt % to about 40 wt %. In an even further aspect, the effective amount is from about 0.01 wt % to about 30 wt %. In a still further aspect, the effective amount is from about 0.01 wt % to about 20 wt %. In yet a further aspect, the effective amount is from about 0.01 wt % to about 10 wt %. In an even further aspect, the effective amount is from about 1 wt % to about 50 wt %. In a still further aspect, the effective amount is from about 5 wt % to about 50 wt %. In yet a further aspect, the effective amount is from about 10 wt % to about 50 wt %. In an even further aspect, the effective amount is from about 20 wt % to about 50 wt %. In a still further aspect, the effective amount is from about 30 wt % to about 50 wt %. In yet a further aspect, the effective amount is from about 40 wt % to about 50 wt %.

In a further aspect, the polymer is a pharmacologically active agent and present in an effective amount. In a further aspect, the effective amount is from about 1 mg to about 1,000 mg per device. In a still further aspect, the effective amount is from about 1 mg to about 750 mg per device. In yet a further aspect, the effective amount is from about 1 mg to about 500 mg per device. In an even further aspect, the effective amount is from about 1 mg to about 250 mg per device. In a still further aspect, the effective amount is from about 1 mg to about 50 mg per device. In yet a further aspect, the effective amount is from about 50 mg to about 1,000 mg per device. In an even further aspect, the effective amount is from about 250 mg to about 1,000 mg per device. In a still further aspect, the effective amount is from about 500 mg to about 1,000 mg per device. In yet a further aspect, the effective amount is from about 750 mg to about 1,000 mg per device.

In various aspects, the controlled-release device is formulated to release the water-swellable gel-forming polymer and/or pharmacologically active agent in vivo and/or in the presence of a change in pH for at least 10 minutes. In a further aspect, release of the polymer and/or active agent continues for at least 30 minutes. In a still further aspect, release of the polymer and/or active agent continues for at least 1 hour. In yet a further aspect, release of the polymer and/or active agent continues for at least 5 hours. In an even further aspect, release of the polymer and/or active agent continues for at least 10 hours. In a still further aspect, release of the polymer and/or active agent continues for at least 12 hours. In yet a further aspect, release of the polymer and/or active agent continues for at least 24 hours. In an even further aspect, release of the polymer and/or active agent continues for at least 2 days. In a still further aspect, release of the polymer and/or active agent continues for at least 3 days. In yet a further aspect, release of the polymer and/or active agent continues for at least 4 days. In an even further aspect, release of the polymer and/or active agent continues for at least 5 days. In a still further aspect, release of the polymer and/or active agent continues for at least 6 days. In yet a further aspect, release of the polymer and/or active agent continues for at least 7 days.

In various aspects, the controlled-release device exhibits release rates of from about 0.001 mg of polymer per day to about 10 mg of polymer per day. In a further aspect, controlled-release device exhibits release rates of from about 0.001 mg to about 10 mg, from about 0.005 mg to about 10 mg, from about 0.01 mg to about 10 mg, from about 0.025 mg to about 10 mg, from about 0.05 mg to about 10 mg, from about 0.075 mg to about 10 mg, from about 0.1 mg to about 10 mg, from about 0.15 mg to about 10 mg, from about 0.2 mg to about 10 mg, from about 0.5 mg to about 10 mg, from about 0.75 mg to about 10 mg, from about 1 mg to about 10 mg, from about 2 mg to about 10, from about 3 mg to about 10, from about 4 mg to about 10 mg, from about 5 mg to about 10 mg, from about 0.001 mg to about 5 mg, from about 0.001 mg to about 4 mg, from about 0.001 mg to about 3 mg, from about 0.001 mg to about 2 mg, from about 0.001 mg to about 1 mg, from about 0.001 mg to about 0.75 mg, from about 0.001 mg, to about 0.5 mg, from about 0.001 mg to about 0.2 mg, from about 0.001 mg to about 0.015 mg, from about 0.001 mg to about 10 mg, from about 0.001 mg to about 0.075 mg, from about 0.001 mg to about 0.05 mg, from about 0.001 mg to about 0.025 mg, from about 0.001 mg to about 0.01 mg, or from about 0.001 mg to about 0.005 mg.

b. Osmotic Agent

In one aspect, the core of the disclosed device comprises an osmotic agent. In a further aspect, the osmotic agent is non-swellable. Examples of osmotic agents include, but are not limited to, magnesium sulfate, magnesium chloride, calcium chloride, sodium chloride, lithium chloride, potassium sulfate, sodium carbonate, sodium sulfite, lithium hydrogen phosphate, lithium sulfate, potassium chloride, sodium hydrogen phosphate, potassium hydrogen phosphate, lithium dihydrogen phosphate, sodium dihydrogen phosphate, potassium dihydrogen phosphate, sodium sulfate, sodium alginate, gelatine, sodium starch glycolate, mannitol, xylitol, urea, urea derivatives, sorbitol, inositol, raffinose, sucrose, glycine, leucine, alanine, methionine, glucose, fructose, lactose, inulin, instant sugar, citric acid, succinic acid, tartaric acid, mannitol, xylitol, sodium acetate, magnesium stearate, sodium benzoate, sodium citrate, sodium ascorbate, and carbohydrates, or mixtures thereof.

In one aspect, the core of the disclosed device optionally comprises an osmotic agent, wherein the core comprises no more than 15 wt % of the osmotic agent. In a further aspect, the core comprises no more than 12 wt % of the osmotic agent. In a still further aspect, the core comprises no more than 10 wt % of the osmotic agent. In yet a further aspect, the core comprises no more than 8 wt % of the osmotic agent. In an even further aspect, the core comprises no more than 6 wt % of the osmotic agent. In a still further aspect, the core comprises no more than 5 wt % of the osmotic agent. In yet a further aspect, the core comprises no more than 4 wt % of the osmotic agent. In an even further aspect, the core comprises no more than 3 wt % of the osmotic agent. In an even further aspect, the core comprises no more than 2 wt % of the osmotic agent. In a still further aspect, the core comprises no more than 1 wt % of the osmotic agent. In yet a further aspect, the core does not comprise an osmotic agent.

In a further aspect, the osmotic agent is non-polymeric.

In a further aspect, the osmotic agent is a low molecular weight polymer. Thus, in various aspects, the osmotic agent has an average molecular weight of about 800,000. In a further aspect, the osmotic agent has an average molecular weight of less than about 800,000. In a still further aspect, the osmotic agent has an average molecular weight of less than about 600,000. In yet a further aspect, the osmotic agent has an average molecular weight of less than about 500,000. In an even further aspect, the osmotic agent has an average molecular weight of less than about 400,000. In a still further aspect, the osmotic agent has an average molecular weight of less than about 300,000. In yet a further aspect, the osmotic agent has an average molecular weight of less than about 200,000. In an even further aspect, the osmotic agent has an average molecular weight of less than about 100,000.

c. Pharmacologically Active Agent

In one aspect, the core of the disclosed device comprises a water-swellable gel-forming polymer and, optionally, a pharmacologically active agent. In a further aspect, the polymer is a pharmacologically active agent.

In one aspect, the core of the disclosed device comprises a water-swellable gel-forming polymer and/or an osmotic agent, and an effective amount of a pharmacologically active agent. In a further aspect, the polymer is a pharmacologically active agent and present in an effective amount.

By “pharmacologically active agent” is meant any agent capable of defending against, or treating, a disease state and/or condition in the human or animal body, or a prodrug thereof. Such agents are intended to be released by diffusion out of the controlled-release device, and may exert their effect either locally or systemically. The active agent(s) may be hydrophilic or lipophilic, organic or inorganic material(s), which are prophylactically or therapeutically active.

The devices of the present invention are capable of delivering macromolecules and hydrophilic small molecules, though they are not limited to such substances. Exemplary macromolecules that may delivered by the present devices include, but are not limited to, sulfated polysaccharides (i.e., carrageenan such as lambda carrageenan, iota carrageenan, and/or kappa carrageenan), proteins (i.e., insulin, HIV-1 envelope proteins, HSV envelope proteins, and therapeutic antibodies), polyacrylic acid, and carbopol, as well as polypyrroles (i.e., those described in K. S. Crowley et al. (2003) Bioorg. Med. Chem. Lett. 13, 1565-1570). In various aspects, the water-swellable gel-forming polymer and the pharmaceutically active agent are the same, such as, e.g., a carrageenan or a mixture of carrageenans.

In various aspects, the disclosed devices are useful for delivering water soluble pharmacologically active agents (i.e., those having a solubility in water of at least about 0.1 mg/mL at physiological pH and temperature) or macromolecules. In various aspects, the pharmacologically active agents have a solubility in water of at least about 0.2 mg/mL. In a further aspect, pharmacologically active agents have a solubility in water of at least about 0.5 mg/mL. In a still further aspect, pharmacologically active agents have a solubility in water of at least about 1 mg/mL. In yet a further aspect, pharmacologically active agents have a solubility in water of at least about 2 mg/mL. In an even further aspect, pharmacologically active agents have a solubility in water of at least about 5 mg/mL. In a still further aspect, pharmacologically active agents have a solubility in water of at least about 10 mg/mL. Excipients that may be formulated with the agents or other substances or may be formulated alone and include, but are not limited to, cellulose, xanthan gum, amino acids, glucosamine, disintegrants, lubricants, carriers, surfactants, fragrances, colorants, and the like.

In a further aspect, the pharmacologically active agent is a pharmaceutically active agent, a biologically active agent, or a nutraceutically active agent.

In various aspects, the pharmacologically active agent is an intravaginally administrable substance. Non-limiting examples of intravaginally administrable substances include microbicides, cervical anesthetics, contraceptive agents, hormones, post-menopausal hormones, antiviral agents, anticancer agents, akt inhibitors, aromatase inhibitors, estrogen receptor modulators, preterm labor drugs, overactive bladder drugs, morning sickness drugs, osteoporosis drugs, antimicrobials, vaccines, and agents for prevention of endometriosis or uterine fibroids.

Other vaginally administrable substances include anticancer drugs; agents for treating dysmenorrhea and/or endometriosis; agents for increasing blood flow to the uterus in preparation for embryo implantation such as sildenafil; agents for inducing labor, cervical ripening, and pregnancy termination; agents for overactive bladder such as oxybutynin; agents for the treatment of preterm labor such as indomethacin; and agents for the treatment of hyperprolactinoma such as bromocriptine. Additional exemplary vaginally administrable substances include agents to treat fungal infections, bacterial vaginosis, and trichomonas infections such as boric acid or borax, metronidazole, clotrimazole, miconazole, terconazole, tinidazole, ‘and clindamycin.

In various aspects, the pharmacologically active agent is blended with the gel-forming polymer. In a further aspect, the pharmacologically active agent is blended with the gel-forming polymer and compressed into a pellet.

In various aspects, the controlled-release device of the present invention may further comprise a second pharmacologically active agent. In a further aspect, the controlled-release device of the present invention comprises at least two pharmacologically active agents. In a still further aspect, the at least two pharmacologically active agents are present in equal amounts. In yet a further aspect, the at least two pharmacologically active agents are present in differing amounts. In an even further aspect, the at least two pharmacologically active agents are present in an effective amount. In a still further aspect, the at least two pharmacologically active agents are present in an individually effective amount.

In various aspects, at least two pharmacologically active agents are blended with the gel-forming polymer. In a further aspect, at least two pharmacologically active agents are blended with the gel-forming polymer and compressed into a pellet.

In various aspects, the pharmacologically active agent is an akt inhibitor. In a further aspect, the akt inhibitor is selected from MK-2206, ARQ 092, perifosine, GDC-0068, and GSK690693.

In various aspects, the pharmacologically active agent is an aromatase inhibitor. In a further aspect, the aromatase inhibitor is selected from Anastrozole, letrozole, exemestane, vorozole, formestane, fadrozole, 1,4,6-androstatrien-3,17-dione, 4-hydroxyandrostenedione, and 1,4,6-androstatrien-3,17-dione.

In various aspects, the pharmacologically active agent is a biologic. In a further aspect, the biologic is a freeze dried organism. In a still further aspect, the biologic is a probiotic. In yet a further aspect, the probiotic is selected from Lactobacillus bulgaricus, Lactobacillus casei subsp. Rhamnosus, Lactobacillus casei subsp. Casei, Lactobacillus salivarius, Lactobacillus brevis, Lactobacillus reuteri, Lactococcus lactis subsp. Lactis, Enterococcus faecium, Lactobacillus plantarum, Streptococcus thermophilus, Bifidobacterium infantis, Bifidobacterium bifidum, Bifidobacterium longum, Saccharomyces boulardii, Lactobacillus acidophilus, Lactobacillus crispatus, whey proteins, lysozyme, lactoferrin, lactoperoxidase, xanthine oxidase, vitamin-binding proteins and immunoglobulins, mannose, oligosaccharides, starches, mannan oligosaccharides, trans-galacto-oligosaccharide, inulin, and fructo-oligosaccharide.

In various aspects, the pharmacologically active agent is a birth control agent. In a further aspect, the birth control agent is selected from ethinyl estradiol, norethindrone, levonorgestrel, ethynodiol diacetate, ethynodiol diacetate, RU486, N9, mifepristone, mifegyne, mifeprex, 17a-ethinyl-levongestrel, 17b-hydroxy-estra-4,9,11-trien-3-one, estradiol, medroxyprogesterone acetate, nestorone, norgestrienone, progesterone, etonogestril (3-keto-desogestrel), progestin, megestrol, etono-progestin alonegestrel, and 17-acetoxy-16-methylene-19-norprogesterone.

In various aspects, the pharmacologically active agent is a fertility agent. In a further aspect, the fertility agent is selected from clomiphene, human chorionic gonadatropin (HCG), Leuprolide acetate, and menotropins.

In various aspects, the pharmacologically active agent prevents a sexually transmitted disease. In a further aspect, the pharmacologically active agent treats a sexually transmitted disease. In a still further aspect, the sexually transmitted disease is selected from HIV-1, HIV-2, AIDS, gonorrhea, chlamydia, trichomonal infections, human papilloma virus (HPV) infections, herpes simplex virus (HSV), syphilis, and genital herpes. In yet a further aspect, the sexually transmitted disease is HIV-1.

In various aspects, the pharmacologically active agent is selected from entry inhibitors, fusion inhibitors, non-nucleoside reverse transcriptase inhibitors (NNRTIs), nucleoside reverse transcriptase inhibitors (NRTIs), nucleotide reverse transcriptase inhibitors, protease inhibitors, NCP7 inhibitors, detergents, surfactants, spermicides, inhibitors of viral adsorption, inhibitors of viral proteases, antivirals, antibiotics, antifungals, anti-inflammatories, antiparasitics, chemotherapeutics, antitoxins, immunotherapeutics, and integrase inhibitors.

In various aspects, the pharmacologically active agent is an anti-HIV agent. In a further aspect, the anti-HIV agent is selected from entry inhibitors, fusion inhibitors, non-nucleoside reverse transcriptase inhibitors (NNRTIs), nucleoside reverse transcriptase inhibitors (NRTIs), nucleotide reverse transcriptase inhibitors, NCP7 inhibitors, protease inhibitors, and integrase inhibitors. In a still further aspect, the entry inhibitor and/or fusion inhibitor is selected from Enfuvirtide (Fuzeon, T-20), AMD11070, PRO542, SCH-C, T-1249, TNX-355, cyanovirin, griffithsen, and maraviroc. In yet a further aspect, the entry inhibitor is selected from a class of lectins. In an even further aspect, the non-nucleoside reverse transcriptase inhibitor is selected from delavirdine (Rescriptor), efavirenz (Sustiva), nevirapine (Viramune), calanolide A, capravirine, epivir, TMC125, adefovir, etravirine, rilpivirine, dapivirine, and lersivirine, and mixtures thereof. In a still further aspect, the nucleoside reverse transcriptase inhibitor and/or nucleotide reverse transcriptase inhibitor is selected from abacavir (Ziagen), didanosine (Videx, ddl), emtricitabine (Emtriva, FTC), lamivudine (Epivir, eTC), stavudine (Zerit, d4t), tenofovir (({[(2R)-1-(6-amino-9H-purin-9-yl)propan-2-yl]oxy}methyl)phosphonic acid), tenofovir disoproxil fumarate, tenofovir alafenamide fumarate, zalcitabine (Hivid, ddc), zidovudine (Retrovir, AZT, ZDR), entecavir, and apricitabine, and mixtures thereof. In a still further aspect, the protease inhibitor is selected from amprenavir (Agenerase), atazanavir (Reyataz), fosamprenavir (Lexiva, 908), indinavir (Crixivan), nelfinavir (Viracept), ritonavir (Norvir), emtriva, saquinavir (Fortovase, Invirase), invirase, agenerase, lopinavir, tipranavir, and darunavir, and mixtures thereof. In yet a further aspect, the integrase inhibitor is selected from elvitegravir, raltegravir, GSK 1265744, GSK-572, and MK-2048, and mixtures thereof. Other anti-HIV agents include, for example, AMD-3100, BMS-806, BMS-793, C31G, carrageenan, CD4-IgG2, cellulose acetate phthalate, zinc salts, cellulose sulphate, cyclodextrins, dextrin-2-sulphate, mAb 2G12, mAb b12, Merck 167, plant lectins, poly naphthalene sulfate, poly sulfo-styrene, PRO2000, PSC-Rantes, SCH-C, SCH-D, T-20, TMC-125, UC-781, UK-427, UK-857, and Viramune, and mixtures thereof.

In various aspects, the pharmacologically active agent is an anti-HSV agent. In a further aspect, the anti-HSV agent targets HSV protease and/or HSV thymidine kinase. In a still further aspect, the anti-HSV agent is a HSV thymidine kinase inhibitor. In yet a further aspect, the anti-HSV agent is selected from acyclovir, ganciclovir, valacyclovir, famciclovir, penciclovir, imiquimod, resiquimod, vidarabine, brivudin, cidofovir, and foscarnet, tenofovir, tenofovir disoproxil fumarate, tenofovir alafenamide fumarate, and mixtures thereof.

In various aspects, the pharmacologically active agent is an anti-HPV agent. In a further aspect, the anti-HPV agent is selected from pyrrole polyamides, lopinavir, and carrageenan, zinc salts, and mixtures thereof.

In various aspects, the pharmacologically active agent is a hormone replacement agent. In a further aspect, the hormone replacement agent is selected from gonadatropin releasing hormone agonists, leuprolide acetate, estrogen, progesterone, testosterone, follicle stimulating hormone (FSH), and progestin.

In various aspects, the pharmacologically active agent is an estrogen receptor modulator. Exemplary estrogen receptor modulators include, but are not limited to, afimoxifene (4-hydroxytamoxifen), arzoxifene, bazedoxifene, clomifene, femarelle (DT56a), lasofoxifene, ormeloxifene, raloxifene, tamoxifen, toremifene, mifepristone (RU486), VA2914, ulipristal, Proellex, Asoprisnil, and CDB-4124.

In various aspects, the pharmacologically active agent is an agent that treats hyperprolactinemia. In a further aspect, the agent that treats hyperprolactinemia is selected from bromocriptine, cabergoline, lisuride, and norprolac.

In various aspects, the pharmacologically active agent is a cervical ripening agent. In a further aspect, the cervical ripening agent is selected from prostaglandin E2, cytotec, laminaria tents, misoprostol and dinoprostone.

In various aspects, the pharmacologically active agent is an antiviral agent. In a further aspect, the antiviral agent is selected from acemannan, acyclovir, acyclovir sodium, adamantanamine, adefovir, adenine arabinoside, alovudine, alvircept sudotox, amantadine hydrochloride, aranotin, arildone, atevirdine mesylate, avridine, cidofovir, cipamfylline, cytarabine hydrochloride, BMS 806, C31G, carrageenan, zinc salts, cellulose sulfate, cyclodextrins, dapivirine, delavirdine mesylate, desciclovir, dextrin 2-sulfate, didanosine, disoxaril, dolutegravir, edoxudine, enviradene, envirozime, etravirine, famciclovir, famotine hydrochloride, fiacitabine, fialuridine, fosarilate, foscarnet sodium, fosfonet sodium, FTC, ganciclovir, ganciclovir sodium, pritelivir, GSK 1265744, 9-2-hydroxy-ethoxy methylguanine, ibalizumab, idoxuridine, interferon, 5-iodo-2′-deoxyuridine, IQP-0528, 1-(cyclopent-3-enylmethyl)-6-(3,5-dimethylbenzoyl)-5-ethylpyrimidine-2,4(1H,3H)-dione, 1-(cyclopentenylmethyl)-6-(3,5-dimethylbenzoyl)-5-isopropylpyrimidine-2,4(1H,3H)-dione, 1-(cyclopent-3-enylmethyl)-6-(3,5-dimethylbenzoyl)-5-isopropylpyrimidine-2,4(1H,3H)-dione, 1-(cyclopropylmethyl)-6-(3,5-dimethylbenzoyl)-5-isopropylpyrimidine-2,4(1H,3H)-dione, 1-(4-benzoyl-2,2-dimethylpiperazin-1-yl)-2-(3H-pyrrolo[2,3-b]pyridin-3-yl)ethane-1,2-dione, kethoxal, lamivudine, lobucavir, maraviroc, memotine pirodavir, penciclovir, raltegravir, ribavirin, rimantadine hydrochloride, rilpivirine (TMC-278), saquinavir mesylate, SCH-C, SCH-D, somantadine hydrochloride, sorivudine, statolon, stavudine, T20, tilorone hydrochloride, TMC120, TMC125, trifluridine, trifluorothymidine, tenofovir, tenofovir alafenamide fumarate, tenofovir disoproxil fumarate, prodrugs of tenofovir, UC-781, UK-427, UK-857, valacyclovir, valacyclovir hydrochloride, pritelivir, vidarabine, vidarabine phosphate, vidarabine sodium phosphate, viroxime, zalcitabine, zidovudine, and zinviroxime.

In various aspects, the pharmacologically active agent is an antibacterial agent. In a further aspect, the antibacterial agent is selected from erythromycin, azithromycin, clarithromycin, telithromycin, penicillin, cephalosporin, carbapenem, imipenem, meropenem, penicillin G, penicillin V, methicillin, oxacillin, cloxacillin, dicloxacillin, nafcillin, ampicillin, amoxicillin, carbenicillin, ticarcillin, meziocillin, piperacillin, azlocillin, temocillin, cepalothin, cephapirin, cephradine, cephaloridine, cefazolin, cefamandole, cefuroxime, cephalexin, cefprozil, cefaclor, loracarbef, cefoxitin, cefmetazole, cefotaxime, ceftizoxime, ceftriaxone, cefoperazone, ceftazidime, cefixime, cefpodoxime, ceftibuten, cefdinir, cefpirome, cefepime, astreonam, gentamycin, chloroquine, cetyl pyridinium chloride, nalidixic acid, oxolinic acid, norfloxacin, pefloxacin, enoxacin, ofloxacin, levofloxacin, ciprofloxacin, temafloxacin, lomefloxacin, fleroxacin, grepafloxacin, sparfloxacin, trovafloxacin, clinafloxacin, gatifloxacin, moxifloxacin, sitafloxacin, ganefloxacin, gemifloxacin, pazufloxacin, para-aminobenzoic acid, sulfadiazine, sulfisoxazole, sulfamethoxazole, sulfathalidine, streptomycin, neomycin, kanamycin, paromycin, gentamicin, tobramycin, amikacin, netilmicin, spectinomycin, sisomicin, dibekalin, sepamicin, tetracycline, chlortetracycline, demeclocycline, minocycline, oxytetracycline, methacycline, doxycycline, rifampicin (also called rifampin), rifapentine, rifabutin, bezoxazinorifamycin, rifaximin, lincomycin, clindamycin, vancomycin, teicoplanin, quinupristin, daflopristin, linezolid, polymyxin, colistin, colymycin, trimethoprim, bacitracin, triclosan, ascorbyl stearate, oleoyl sarcosine, dioctyl sulfosuccinate, and phosphonomycin.

In various aspects, the pharmacologically active agent is an antifungal agent. In a further aspect, the antifungal agent is selected from an azole, polyene, echinocandin, and pradimicin. In a still further aspect, the antifungal agent is selected from fluconazole, isavuconazole, itraconazole, ketoconazole, miconazole, clortrimazole, voriconazole, posaconazole, rovuconazole, natamycin, lucensomycin, nystatin, amphotericin B, CANCIDAS®, beanomicins, nikkomycins, sordarins, allylamines, triclosan, piroctone, fenpropimorph, boric acid or borax, metronidazole, terconazole, tinidazole, and clindamycin, and terbinafine.

In various aspects, the pharmacologically active agent is selected from a lubricant and a moisturizer. In a further aspect, the pharmacologically active agent is a lubricant. In a still further aspect, the lubricant is selected from high molecular weight polyethylene glycols and glycol esters, triglycerides, glycerin, polycarbophil and polysorbates, pyridine, squalene, urea, complex alcohols, aldehydes, ketones, stearic acid, stearate, isopropyl palmitate, petrolatum, aloe barbadensis (Aloe Vera) leaf juice, cucumus sativus extract, helianthus annulus seed oil, soybean sterol, vitamin E acetate, vitamin A palmitate, provitamin B5, sodium acrylate/acryloyldimethyl taurate copolymer, dimethicone, glyceryl stearate, ceylalcohol, lecithin, mineral water, sodium PCA, potassium lactate, collagen, aminoacids, triethanolamine, DMDM hydantoin, iodopropynyl, butylcarbamate, disodium EDTA, and titanium dioxide.

In various aspects, the pharmacologically active agent is an agent that treats uterine fibroids. In a further aspect, the agent that treats uterine fibroids binds to the GnRH receptor. In a still further aspect, the agent that treats uterine fibroids is a GnRH agonist. In yet a further aspect, the GnRH agonist is selected from leuprolide, dislorelin, triptorelin, leoprorelin, buserelin, nafarelin, goserelin, [Dlys6]GnRH, [Dala]GnRH, avorelin, histerelin, PTL 03301, AN 207, TX 397, AN 201, and SPD 424.

In various aspects, the pharmacologically active agent is an anti-cancer agent. In a further aspect, the anti-cancer agent is selected from fluorouracil, cisplatin, doxorubicin, leuprolide acetate, and paclitaxel. In a still further aspect, the anti-cancer agent binds to the GnRH receptor. In yet a further aspect, the anti-cancer agent is a GnRH agonist. In an even further aspect, the GnRH agonist is selected from leuprolide, dislorelin, triptorelin, leoprorelin, buserelin, nafarelin, goserelin, [Dlys6]GnRH, [Dala]GnRH, avorelin, histerelin, PTL 03301, AN 207, TX 397, AN 201, and SPD 424.

In various aspects, the pharmacologically active agent is an antiemetic. In a further aspect, the antiemetic is selected from an antihistamine, benzamide, butyrophenone, benzodiazepine, cannabinoid, corticosteroid, phenothiazine, serotonin antagonist, and neurokinin-1-receptor antagonist. In a still further aspect, the antiemetic is a selected from bromocriptine, diphenhydramine, hydroxyzine, meclizine, metoclopramide, haloperidol, droperidol, lorazepam, alprazolam, dronabinol, dexamethasone, methylprednisolone, prochlorperazine, promethazine, chlorpromazine, ondansetron, dolasetron, granisetron, and aprepitant.

In various aspects, the pharmacologically active agent is an agent that treats endometriosis. In a further aspect, the agent that treats endometriosis is selected from terbutaline, Abarelix, SERMs (Selective Estrogen Receptor Modulators), Extracellular Matrix Modulators, and RU-486. In a still further aspect, the agent that treats endometriosis binds to the GnRH receptor. In yet a further aspect, the agent that treats endometriosis is a GnRH agonist. In an even further aspect, the GnRH agonist is selected from leuprolide, dislorelin, triptorelin, leoprorelin, buserelin, nafarelin, goserelin, [Dlys6]GnRH, [Dala]GnRH, avorelin, histerelin, PTL 03301, AN 207, TX 397, AN 201, and SPD 424.

In various aspects, the pharmacologically active agent is an agent for restoration and/or maintenance of healthy vaginal microflora. Exemplary agents include probiotics, Lactobacillus bulgaricus, Lactobacillus casei subsp. Rhamnosus, Lactobacillus casei subsp. Casei, Lactobacillus salivarius, Lactobacillus brevis, Lactobacillus reuteri, Lactococcus lactis subsp. Lactis, Enterococcus faecium, Lactobacillus plantarum, Streptococcus thermophilus, Bifidobacterium infantis, Bifidobacterium bifidum, Bifidobacterium longum, Saccharomyces boulardii, Lactobacillus acidophilus, whey proteins, lysozyme, lactoferrin, lactoperoxidase, xanthine oxidase, vitamin-binding proteins and immunoglobulins, mannose, oligosaccharides, starches, mannan oligosaccharides, trans-galacto-oligosaccharide, inulin, and fructo-oligosaccharide.

In a further aspect, the pharmacologically active agent is present in an effective amount. In a still further aspect, the effective amount is from about 0.01 wt % to about 50 wt %. In yet a further aspect, the effective amount is from about 0.01 wt % to about 40 wt %. In an even further aspect, the effective amount is from about 0.01 wt % to about 30 wt %. In a still further aspect, the effective amount is from about 0.01 wt % to about 20 wt %. In yet a further aspect, the effective amount is from about 0.01 wt % to about 10 wt %. In an even further aspect, the effective amount is from about 1 wt % to about 50 wt %. In a still further aspect, the effective amount is from about 5 wt % to about 50 wt %. In yet a further aspect, the effective amount is from about 10 wt % to about 50 wt %. In an even further aspect, the effective amount is from about 20 wt % to about 50 wt %. In a still further aspect, the effective amount is from about 30 wt % to about 50 wt %. In yet a further aspect, the effective amount is from about 40 wt % to about 50 wt %.

In a further aspect, the effective amount is from about 1 mg to about 1,000 mg per device. In a still further aspect, the effective amount is from about 1 mg to about 750 mg per device. In yet a further aspect, the effective amount is from about 1 mg to about 500 mg per device. In an even further aspect, the effective amount is from about 1 mg to about 250 mg per device. In a still further aspect, the effective amount is from about 1 mg to about 50 mg per device. In yet a further aspect, the effective amount is from about 50 mg to about 1,000 mg per device. In an even further aspect, the effective amount is from about 250 mg to about 1,000 mg per device. In a still further aspect, the effective amount is from about 500 mg to about 1,000 mg per device. In yet a further aspect, the effective amount is from about 750 mg to about 1,000 mg per device.

In various aspects, the controlled-release device is formulated to release the polymer and/or pharmacologically active agent in vivo and/or in the presence of a change in pH for at least 10 minutes. In a further aspect, release of the polymer and/or active agent continues for at least 30 minutes. In a still further aspect, release of the polymer and/or active agent continues for at least 1 hour. In yet a further aspect, release of the polymer and/or active agent continues for at least 5 hours. In an even further aspect, release of the polymer and/or active agent continues for at least 10 hours. In a still further aspect, release of the polymer and/or active agent continues for at least 12 hours. In yet a further aspect, release of the polymer and/or active agent continues for at least 24 hours. In an even further aspect, release of the polymer and/or active agent continues for at least 2 days. In a still further aspect, release of the polymer and/or active agent continues for at least 3 days. In yet a further aspect, release of the polymer and/or active agent continues for at least 4 days. In an even further aspect, release of the polymer and/or active agent continues for at least 5 days. In a still further aspect, release of the polymer and/or active agent continues for at least 6 days. In yet a further aspect, release of the polymer and/or active agent continues for at least 7 days.

In various aspects, the controlled-release device exhibits release rates of from about 0.001 mg of polymer and/or pharmacologically active agent per day to about 10 mg of pharmacologically active agent per day. In a further aspect, controlled-release device exhibits release rates of from about 0.001 mg to about 10 mg, from about 0.005 mg to about 10 mg, from about 0.01 mg to about 10 mg, from about 0.025 mg to about 10, from about 0.05 mg to about 10 mg, from about 0.075 mg to about 10 mg, from about 0.1 mg to about 10 mg, from about 0.15 mg to about 10 mg, from about 0.2 mg to about 10 mg, from about 0.5 mg to about 10 mg, from about 0.75 mg to about 10 mg, from about 1 mg to about 10 mg, from about 2 mg to about 10, from about 3 mg to about 10, from about 4 mg to about 10 mg, from about 5 mg to about 10 mg, from about 0.001 mg to about 5 mg, from about 0.001 mg to about 4 mg, from about 0.001 mg to about 3 mg, from about 0.001 mg to about 2 mg, from about 0.001 mg to about 1 mg, from about 0.001 mg to about 0.75 mg, from about 0.001 mg, to about 0.5 mg, from about 0.001 mg to about 0.2 mg, from about 0.001 mg to about 0.015 mg, from about 0.001 mg to about 10 mg, from about 0.001 mg to about 0.075 mg, from about 0.001 mg to about 0.05 mg, from about 0.001 mg to about 0.025 mg, from about 0.001 mg to about 0.01 mg, or from about 0.001 mg to about 0.005 mg.

2. Water-Insoluble Coating

In one aspect, the controlled-release device comprises a substantially inelastic, water-insoluble coating substantially enclosing the core, wherein at least a portion of the coating comprises a semipermeable membrane. In various aspects, the coating is water resistant.

In one aspect, the controlled-release device of the present invention comprises a water-insoluble coating substantially enclosing the core, wherein at least a portion of the coating comprises a pH-responsive material. In a further aspect, the pH-responsive material is water-insoluble at a pH of less than about 6. In a still further aspect, the pH-responsive material is water-insoluble at a pH of less than about 5. In yet a further aspect, the pH-responsive material is water-insoluble at a pH of less than about 4. In an even further aspect, the pH-responsive material is water-soluble at a pH of greater than about 6. In a still further aspect, the pH-responsive material is water-soluble at a pH of greater than about 7. In yet a further aspect, the pH-responsive material is water-soluble at a pH of greater than about 8.

In a further aspect, inelastic is unable to stretch. In a still further aspect, inelastic is unable to stretch by more than 50%. In yet a further aspect, inelastic is able to stretch at least 50% but unable to retract by more than 30%.

In a further aspect, water-insoluble is not susceptible to being dissolved in water.

In various aspects, at least a portion of the coating comprises a semipermeable membrane. In a further aspect, the semipermeable membrane forms substantially all of the coating. In a still further aspect, substantially is completely. In yet a further aspect, substantially is generally, typically, or approximately.

In various aspects, the semipermeable membrane comprises a film-forming polymer. Film-forming polymers that may be used in the device include any biocompatible polymers that swell on contact with water, i.e., anionic or cationic polymers (including zwitterionic) or nonionic water soluble polymers. Such polymers include, but are not limited to, cellulose esters (i.e., cellulose acetate, cellulose acetate phthalate, cellulose acetate butyrate, cellulose acetate propionate), acrylate co-polymers commercially available as Eudragit®, polymers manufactured by Evonik Industries and other polymers capable of forming semipermeable membranes used in osmosis or reverse osmosis known in the art.

Film-forming polymers of the present invention may be pH-responsive polymers, insoluble below pH 6 and soluble in aqueous media above pH 6. Thus, in various aspects, the dissolution rate of the film-forming polymer is less than 1 min. In a further aspect, the dissolution rate of the film-forming polymer is from about 1 min to about 15 min. In a still further aspect, the dissolution rate of the film-forming polymer is from about 1 min to about 10 min. In yet a further aspect, the dissolution rate of the film-forming polymer is from about 1 min to about 8 min. In an even further aspect, the dissolution rate of the film-forming polymer is from about 1 min to about 6 min. In a still further aspect, the dissolution rate of the film-forming polymer is from about 1 min to about 4 min. In yet a further aspect, the dissolution rate of the film-forming polymer is from about 1 min to about 2 min. In an even further aspect, the dissolution rate of the film-forming polymer is from about 2 min to about 10 min. In a still further aspect, the dissolution rate of the film-forming polymer is from about 4 min to about 10 min. In yet a further aspect, the dissolution rate of the film-forming polymer is from about 6 min to about 10 min. In an even further aspect, the dissolution rate of the film-forming polymer is from about 8 min to about 10 min.

In a further aspect, the semipermeable membrane allows influx of a liquid from the exterior of the device to the interior of the device, while at the same time allowing release of the polymer in the core by osmotic pumping through the orifice in the semipermeable membrane.

In various aspects, the semipermeable membrane further comprises pore-forming agents (e.g., porogens). Examples of pore-forming agents include, but are not limited to, salts of sodium or potassium and mono- and di-saccharides that dissolve upon hydration, thereby increasing the porosity of the membrane.

In various aspects, the device of the present invention optionally further comprises a partial coating of a mucoadhesive polymer. Mucoadhesive polymers are typically hydrophilic, and upon moistening, swell and become adhesive. Examples of mucoadhesive polymers that may be employed in the present invention include but are not limited to acrylic acid monomers such as polyacrylic acid and any of its pharmaceutically acceptable salts; copolymers of acrylic acid and methacrylic acid, styrene, or vinyl ethers; vinyl polymers such as polyhydroxyethyl acrylate, polyhydroxyethyl methacrylate, polyvinyl alcohol, and polyvinyl pyrrolidone; cellulosic derivatives such as methyl cellulose, ethyl cellulose, hydroxyethyl cellulose, hydroxypropyl cellulose, hydroxypropylmethyl cellulose, and carboxymethyl cellulose; polysaccharides such as alginic acid, sodium alginate, and tragacanth gum; collagen; polymeratin; Carbopols; Gantrez™ (Ashland); and any combination thereof.

In a further aspect, at least a portion of the coating comprises a pH-responsive material. In a still further aspect, the pH-responsive material is water-insoluble at a pH of less than about 6, and wherein the pH-responsive material is water-soluble at a pH of greater than about 6. In yet a further aspect, the pH-responsive material is water-insoluble at a pH of less than about 5, and wherein the pH-responsive material is water-soluble at a pH of greater than about 6. In an even further aspect, the pH-responsive material is water-insoluble at a pH of less than about 4, and wherein the pH-responsive material is water-soluble at a pH of greater than about 6. In a still further aspect, the pH-responsive material is water-insoluble at a pH of less than about 6, and wherein the pH-responsive material is water-soluble at a pH of greater than about 7. In yet a further aspect, the pH-responsive material is water-insoluble at a pH of less than about 6, and wherein the pH-responsive material is water-soluble at a pH of greater than about 8. In an even further aspect, the pH-responsive material is water-insoluble at a pH of less than about 5, and wherein the pH-responsive material is water-soluble at a pH of greater than about 7.

3. Orifice

In one aspect, the controlled-release device of the present invention comprises at least one orifice in the coating, wherein the orifice is positioned and dimensioned to allow controlled-release of the gel-forming polymer from the core in response to swelling of the polymer. In a further aspect, the orifice is defined by the coating. In a still further aspect, the orifice is located in the membrane. By “orifice” is meant a hole through which the sustained release formulation may directly pass into the environment without having to diffuse through a polymeric barrier. The chamber is sealed except for the orifice(s) through which the formulation passes. The orifice must also be sufficiently small such that the formulation is released over the desired time period of e.g, days or weeks. The orifice may have any suitable shape, including but not limited to round, square, or polygonal. In various aspects, the chamber has a single orifice with a diameter of about 0.1 mm to about 4 mm. In a further aspect, the orifice has two, three, four, five, or more orifices. In a still further aspect, the orifice(s) have a diameter of about 0.5 mm to about 4 mm, about 1 to about 4 mm, about 1 to about 3 mm, or about 1 to about 2 mm in diameter.

The size and shape of orifices of the present technology may vary but are typically designed to hold from 1 to 1,000 mg of the controlled-release formulation. In various aspects, the orifice is cylindrical. Such orifices may have a diameter ranging from about 3 to about 15 mm and a height ranging from about 0.1 to about 1 mm. In a further aspect, the diameter ranges from about 5 to about 10 mm and the height ranges from about 0.2 to about 0.8 mm. In a still further aspect, the orifice is a solid object made with the same curvature of the device.

A variety of devices may be fitted with an orifice as described herein, including but not limited to, an intravaginal ring, tampon, or pessary. The controlled-release device may include one or two or more chambers (i.e., two, three, four, five, or six), each may include the same controlled-release formulation or one or more different controlled-release formulations. In various aspects, the devices include two or three chambers, each including an orifice and containing a controlled-release formulation.

In various aspects, the orifice receives the gel-forming polymer from the core. In a further aspect, the orifice extends radially outwardly from the core through the coating. Thus, the orifice provides communication between the core and the environment surrounding the device.

### C. METHODS OF MAKING A CONTROLLED-RELEASE DEVICE

In one aspect, the invention relates to a method of making a controlled-release device, the method comprising the steps of: a) providing a core comprising a water-swellable gel-forming polymer, optionally, an osmotic agent, and, optionally, a pharmacologically active agent; b) substantially enclosing the core within a substantially inelastic, water-insoluble coating, wherein at least a portion of the coating comprises a semipermeable membrane; and c) creating at least one orifice in the coating, wherein the core comprises no more than 15 wt % of the osmotic agent. In a further aspect, the substantially enclosing step comprises spray coating. In a still further aspect, the creating step is laser cutting.

In a further aspect, the substantially enclosing step and the creating step are performed sequentially.

In a further aspect, the core comprises no more than 15 wt % of the osmotic agent. In a still further aspect, the core comprises no more than 12 wt % of the osmotic agent. In yet a further aspect, the core comprises no more than 10 wt % of the osmotic agent. In an even further aspect, the core comprises no more than 8 wt % of the osmotic agent. In a still further aspect, the core comprises no more than 6 wt % of the osmotic agent. In yet a further aspect, the core comprises no more than 5 wt % of the osmotic agent. In an even further aspect, the core comprises no more than 4 wt % of the osmotic agent. In an even further aspect, the core comprises no more than 3 wt % of the osmotic agent. In a still further aspect, the core comprises no more than 2 wt % of the osmotic agent. In yet a further aspect, the core comprises no more than 1 wt % of the osmotic agent. In an even further aspect, the core does not comprise an osmotic agent.

In a further aspect, the method further comprises a pH-responsive material covering the core. In a still further aspect, the pH-responsive material is water-insoluble at a pH of less than about 6. In yet a further aspect, the pH-responsive material is water-insoluble at a pH of less than about 5. In an even further aspect, the pH-responsive material is water-insoluble at a pH of less than about 4. In a still further aspect, the pH-responsive material is water-soluble at a pH of greater than about 6. In yet a further aspect, the pH-responsive material is water-soluble at a pH of greater than about 7. In an even further aspect, the pH-responsive material is water-soluble at a pH of greater than about 8.

### D. DELIVERY METHODS

In one aspect, the invention relates to methods of delivering pharmacologically active agents, the method comprising contacting a controlled-release device with vaginal membrane tissue, wherein the device comprises: a) a core comprising: i) a water-swellable gel-forming polymer and/or an osmotic agent; and ii) an effective amount of a pharmacologically active agent; b) a substantially inelastic, water-insoluble coating substantially enclosing the core, wherein at least a portion of the coating comprises a semipermeable membrane; and c) at least one orifice in the coating, wherein the orifice is positioned and dimensioned to allow controlled-release of the pharmacologically active agent from the core.

In a further aspect, the core comprises no more than 15 wt % of the osmotic agent. In a still further aspect, the core comprises no more than 12 wt % of the osmotic agent. In yet a further aspect, the core comprises no more than 10 wt % of the osmotic agent. In an even further aspect, the core comprises no more than 8 wt % of the osmotic agent. In a still further aspect, the core comprises no more than 6 wt % of the osmotic agent. In yet a further aspect, the core comprises no more than 5 wt % of the osmotic agent. In an even further aspect, the core comprises no more than 4 wt % of the osmotic agent. In a still further aspect, the core comprises no more than 3 wt % of the osmotic agent. In yet a further aspect, the core comprises no more than 2 wt % of the osmotic agent. In an even further aspect, the core comprises no more than 1 wt % of the osmotic agent. In a still further aspect, the core does not comprise an osmotic agent.

In a further aspect, the device of the disclosed delivery method further comprises a pH-responsive material covering the core. In a still further aspect, the pH-responsive material is water-insoluble at a pH of less than about 6. In yet a further aspect, the pH-responsive material is water-insoluble at a pH of less than about 5. In an even further aspect, the pH-responsive material is water-insoluble at a pH of less than about 4. In a still further aspect, the pH-responsive material is water-soluble at a pH of greater than about 6. In yet a further aspect, the pH-responsive material is water-soluble at a pH of greater than about 7. In an even further aspect, the pH-responsive material is water-soluble at a pH of greater than about 8.

In various aspects, the invention relates to a delivery method comprising the step of placing a disclosed controlled-release device in contact with mucous membrane tissue of a mammal.

In various aspects, the invention relates to a delivery method comprising the step of placing a controlled-release device prepared by a disclosed method of making in contact with mucous membrane tissue of a mammal.

In a further aspect, the tissue is selected from oral, nasal, vaginal, and rectal. In a still further aspect, the tissue is vaginal.

In a further aspect, the mammal is a human.

### E. METHODS OF USING A CONTROLLED-RELEASE DEVICE

The device disclosed herein is useful for administering a lubricant, preventing fertility, promoting fertility, preventing a sexually transmitted disease, treating a sexually transmitted disease, hormone replacement, cervical ripening, treating a mucosal infection, delivering a biologic, treating vaginal dryness, treating uterine fibroids, treating reproductive cancers, treating nausea gravidarum, and treating endometriosis. Thus, in various aspects, disclosed are methods for the aforementioned uses, the methods comprising contacting a controlled-release device with mucous membrane tissue, wherein the device comprises: a) a core comprising: i) a water-swellable gel-forming polymer; ii) an effective amount of a pharmacologically active agent; and iii) optionally, an osmotic agent; b) a substantially inelastic, water-insoluble coating substantially enclosing the core, wherein at least a portion of the coating comprises a semipermeable membrane; and c) at least one orifice in the coating, wherein the orifice is positioned and dimensioned to allow controlled-release of the polymer from the core in response to swelling of the polymer, wherein the core is comprises no more than 15 wt % of the osmotic agent.

In a further aspect, the tissue is selected from oral, nasal, vaginal, and rectal.

In a further aspect, the core comprises no more than 15 wt % of the osmotic agent. In a still further aspect, the core comprises no more than 12 wt % of the osmotic agent. In yet a further aspect, the core comprises no more than 10 wt % of the osmotic agent. In an even further aspect, the core comprises no more than 8 wt % of the osmotic agent. In a still further aspect, the core comprises no more than 6 wt % of the osmotic agent. In yet a further aspect, the core comprises no more than 5 wt % of the osmotic agent. In an even further aspect, the core comprises no more than 4 wt % of the osmotic agent. In an even further aspect, the core comprises no more than 3 wt % of the osmotic agent. In a still further aspect, the core comprises no more than 2 wt % of the osmotic agent. In yet a further aspect, the core comprises no more than 1 wt % of the osmotic agent. In an even further aspect, the core does not comprise an osmotic agent.

In various further aspects, disclosed are methods for the aforementioned uses, the methods comprising contacting a controlled-release device with vaginal membrane tissue, wherein the device comprises: a) a core comprising: i) a water-swellable gel-forming polymer and/or an osmotic agent; and ii) an effective amount of a pharmacologically active agent; b) a substantially inelastic, water-insoluble coating substantially enclosing the core, wherein at least a portion of the coating comprises a semipermeable membrane; and c) at least one orifice in the coating, wherein the orifice is positioned and dimensioned to allow controlled-release of the pharmacologically active agent from the core.

In a further aspect, the polymer is a pharmacologically active agent and present in an effective amount.

In a further aspect, the effective amount is from about 0.01 wt % to about 50 wt %. In a still further aspect, the effective amount is from about 0.01 wt % to about 40 wt %. In yet a further aspect, the effective amount is from about 0.01 wt % to about 30 wt %. In an even further aspect, the effective amount is from about 0.01 wt % to about 20 wt %. In a still further aspect, the effective amount is from about 0.01 wt % to about 10 wt %. In yet a further aspect, the effective amount is from about 1 wt % to about 50 wt %. In an even further aspect, the effective amount is from about 5 wt % to about 50 wt %. In a still further aspect, the effective amount is from about 10 wt % to about 50 wt %. In yet a further aspect, the effective amount is from about 20 wt % to about 50 wt %. In an even further aspect, the effective amount is from about 30 wt % to about 50 wt %. In a still further aspect, the effective amount is from about 40 wt % to about 50 wt %.

In a further aspect, the effective amount is from about 1 mg to about 1,000 mg per device. In a still further aspect, the effective amount is from about 1 mg to about 750 mg per device. In yet a further aspect, the effective amount is from about 1 mg to about 500 mg per device. In an even further aspect, the effective amount is from about 1 mg to about 250 mg per device. In a still further aspect, the effective amount is from about 1 mg to about 50 mg per device. In yet a further aspect, the effective amount is from about 50 mg to about 1,000 mg per device. In an even further aspect, the effective amount is from about 250 mg to about 1,000 mg per device. In a still further aspect, the effective amount is from about 500 mg to about 1,000 mg per device. In yet a further aspect, the effective amount is from about 750 mg to about 1,000 mg per device.

In various aspects, the controlled-release device is formulated to release the polymer and/or pharmacologically active agent in vivo and/or in the presence of a change in pH for at least 10 minutes. In a further aspect, release of the polymer and/or active agent continues for at least 30 minutes. In a still further aspect, release of the polymer and/or active agent continues for at least 1 hour. In yet a further aspect, release of the polymer and/or active agent continues for at least 5 hours. In an even further aspect, release of the polymer and/or active agent continues for at least 10 hours. In a still further aspect, release of the polymer and/or active agent continues for at least 12 hours. In yet a further aspect, release of the polymer and/or active agent continues for at least 24 hours. In an even further aspect, release of the polymer and/or active agent continues for at least 2 days. In a still further aspect, release of the polymer and/or active agent continues for at least 3 days. In yet a further aspect, release of the polymer and/or active agent continues for at least 4 days. In an even further aspect, release of the polymer and/or active agent continues for at least 5 days. In a still further aspect, release of the polymer and/or active agent continues for at least 6 days. In yet a further aspect, release of the polymer and/or active agent continues for at least 7 days.

In various aspects, the controlled-release device exhibits release rates of from about 0.001 mg of pharmacologically active agent and/or polymer per day to about 10 mg of pharmacologically active agent and/or polymer per day. In a further aspect, controlled-release device exhibits release rates of from about 0.001 mg to about 10 mg, from about 0.005 mg to about 10 mg, from about 0.01 mg to about 10 mg, from about 0.025 mg to about 10, from about 0.05 mg to about 10 mg, from about 0.075 mg to about 10 mg, from about 0.1 mg to about 10 mg, from about 0.15 mg to about 10 mg, from about 0.2 mg to about 10 mg, from about 0.5 mg to about 10 mg, from about 0.75 mg to about 10 mg, from about 1 mg to about 10 mg, from about 2 mg to about 10, from about 3 mg to about 10, from about 4 mg to about 10 mg, from about 5 mg to about 10 mg, from about 0.001 mg to about 5 mg, from about 0.001 mg to about 4 mg, from about 0.001 mg to about 3 mg, from about 0.001 mg to about 2 mg, from about 0.001 mg to about 1 mg, from about 0.001 mg to about 0.75 mg, from about 0.001 mg, to about 0.5 mg, from about 0.001 mg to about 0.2 mg, from about 0.001 mg to about 0.015 mg, from about 0.001 mg to about 10 mg, from about 0.001 mg to about 0.075 mg, from about 0.001 mg to about 0.05 mg, from about 0.001 mg to about 0.025 mg, from about 0.001 mg to about 0.01 mg, or from about 0.001 mg to about 0.005 mg.

In a further aspect, the device of the disclosed method further comprises a pH-responsive material covering the core. In a still further aspect, the pH-responsive material is water-insoluble at a pH of less than about 6. In yet a further aspect, the pH-responsive material is water-insoluble at a pH of less than about 5. In an even further aspect, the pH-responsive material is water-insoluble at a pH of less than about 4. In a still further aspect, the pH-responsive material is water-soluble at a pH of greater than about 6. In yet a further aspect, the pH-responsive material is water-soluble at a pH of greater than about 7. In an even further aspect, the pH-responsive material is water-soluble at a pH of greater than about 8.

In various aspects, the disclosed treatment methods can be applied to a subject. In a further aspect, the subject is a mammal. In a still further aspect, the mammal is a human.

1. Administering a Lubricant

In one aspect, the invention relates to methods of administering a lubricant, the method comprising contacting a controlled-release device with vaginal membrane tissue, wherein the device comprises: a) a core comprising: i) a water-swellable gel-forming polymer and/or an osmotic agent; and ii) at least one lubricant, b) a substantially inelastic, water-insoluble coating substantially enclosing the core, wherein at least a portion of the coating comprises a semipermeable membrane; and c) at least one orifice in the coating, wherein the orifice is positioned and dimensioned to allow controlled-release of the lubricant from the core. Examples of lubricants include, but are not limited to, pyridine, squalene, urea, complex alcohols, aldehydes, ketones, stearic acid, stearate, isopropyl palmitate, petrolatum, aloe barbadensis (Aloe Vera) leaf juice, cucumus sativus extract, helianthus annulus seed oil, soybean sterol, vitamin E acetate, vitamin A palmitate, provitamin B5, sodium acrylate/acryloyldimethyl taurate copolymer, dimethicone, glyceryl stearate, ceylalcohol, lecithin, mineral water, sodium PCA, potassium lactate, collagen, aminoacids, triethanolamine, DMDM hydantoin, iodopropynyl, butylcarbamate, disodium EDTA, and titanium dioxide.

The lubricant may be aqueous or non-aqueous. For example, the lubricant can be water, a hypo-osmolar water or solution, an aqueous solution, a hyper-osmotic water or solution, an iso-osmotic water or solution, an aqueous solution, and a polymer. Thus, in various aspects, the lubricant comprises water in an amount of at least 90 wt %. In a further aspect, the lubricant comprises water in an amount of at least 95 wt %. In a still further aspect, the lubricant comprises water in an amount of at least 96 wt %. In yet a further aspect, the lubricant comprises water in an amount of at least 97 wt %. In an even further aspect, the lubricant comprises water in an amount of at least 98 wt %. In a still further aspect, the lubricant comprises water in an amount of at least 99 wt %. In yet a further aspect, the lubricant comprises water in an amount of 100 wt %.

In various aspects, the aqueous lubricant is iso-osmolar or hypo-osmolar and may include ions such as potassium, sodium, chloride and phosphate. The ions can be present, for example, in an amount from about 0.1 wt % to about 0.75 wt %, about 0.1 wt % to about 0.50 wt %, 0.1 wt % to about 0.25 wt %, 0.25 wt % to about 0.75 wt %, or about 0.50 wt % to about 0.75 wt %.

In various aspects, the lubricant may be buffered, optionally at an acidic pH. Thus, the lubricant may have a pH of from about 3 to about 8, about 3 to about 6, about 3 to about 5, about 3 to about 4.5, or about 3.5 to about 4.5.

The lubricant may include vaginal fluid simulant, lactic acid in an amount from about 5 mM to about 50 mM, an acetic acid buffer in an amount from about 10 mM to about 30 mM at a pH of from about 3.5 to about 5.0, and, optionally, glucose in an amount from about 5 mM to about 50 mM. In a further aspect, the aqueous lubricant may include vaginal fluid simulant, lactic acid in an amount from about 20 mM to about 30 mM, acetic acid buffer in an amount from about 15 mM to about 25 mM at a pH of from about 3.5 to about 5.0, and, optionally, glucose in an amount from about 20 mM to about 30 mM. The lubricant may be free of steroids or may be free of any active pharmaceutical ingredient (i.e., those ingredients that have a therapeutic effect as opposed to a non-therapeutic biological effect).

In various aspects, the lubricant may include a variety of additives. Exemplary additives may include one or more salts, nonaqueous solvents (i.e., propylene glycol or glycerol), acids such as C1-C8 carboxylic acids (i.e., lactic acid or acetic acid), glucose, antioxidants (i.e., BHT or ascorbic acid), preservatives (i.e., sorbital, sorbic acid, parabens, EDTA, sodium benzoate, or tocopherol), surfactants (i.e., polysorbate 20, polysorbate 60, or sorbate salts), fragrance, flavoring agents, and sweeteners (i.e., saccharine or aspartamate). Additional additives that may be used include pyridine, squalene, urea, complex alcohols, aldehydes, ketones, stearic acid, stearate, isopropyl palmitate, petrolatum, aloe barbadensis (Aloe Vera) leaf juice, cucumus sativus extract, helianthus annulus seed oil, soybean sterol, vitamin E acetate, vitamin A palmitate, provitamin B5, sodium acrylate/acryloyldimethyl taurate copolymer, dimethicone, glyceryl stearate, ceylalcohol, lecithin, mineral water, sodium PCA, potassium lactate, collagen, aminoacids, triethanolamine, DMDM hydantoin, iodopropynyl, butylcarbamate, disodium EDTA, titanium dioxide. The additives may be added at a concentration such that the aqueous lubricant is hypo-osmotic, hyper-osmotic or iso-osmolar in comparison to bodily fluids, blood, or tissue. As used herein, the term “hypo-osmotic lubricant” means that the osmolality of the lubricant is less than that of the bodily fluid, blood, or tissue fluid. In contrast, the term “hyper-osmotic lubricant,” as used herein, means that the osmolality is greater than that of the bodily fluid, blood, or tissue fluid. The term “iso-osmotic lubricant,” as used herein, means that the osmolality is essentially the same as the osmolality of the bodily fluid, blood, or tissue fluid.

Hyper-osmotic lubricants may be aqueous or non-aqueous. Such non-aqueous lubricants may be water-soluble (i.e., at least 1 mg/mL at 25° C.). Hyper-osmotic lubricants may be prepared from appropriate concentrations of various agents including, but not limited to, glycerol, polyethylene glycol, propylene glycol, carrageenan (i.e., sulfated polysaccharides), zinc salts, other lubricating or hydrating substances, salts, and hyper-osmotic aqueous agents, and the like. Thus, in various aspects, the hyper-osmotic lubricant comprises glycerol in an amount from about 4 wt % to about 100 wt %, from about 4 wt % to about 75 wt %, from about 4 wt % to about 50 wt %, from about 4 wt % to about 25 wt %, from about 4 wt % to about 10 wt %, from about 10 wt % to about 100 wt %, from about 25 wt % to about 100 wt %, from about 50 wt % to about 100 wt %, or from about 75 wt % to about 100 wt %. In further various aspects, the hyper-osmotic lubricant comprises propylene glycol in an amount from about 3 wt % to about 100 wt %, from about 3 wt % to about 75 wt %, from about 3 wt % to about 50 wt %, from about 3 wt % to about 25 wt %, from about 3 wt % to about 10 wt %, from about 10 wt % to about 100 wt %, from about 25 wt % to about 100 wt %, from about 50 wt % to about 100 wt %, or from about 75 wt % to about 100 wt %.

In a further aspect, the tissue is selected from oral, nasal, vaginal, and rectal.

In a further aspect, the core comprises no more than 15 wt % of the osmotic agent. In a still further aspect, the core comprises no more than 10 wt % of the osmotic agent. In yet a further aspect, the core comprises no more than 5 wt % of the osmotic agent. In an even further aspect, the core does not comprise an osmotic agent.

In a further aspect, the lubricant is water-soluble. In a still further aspect, the lubricant is not water-soluble.

In a further aspect, the polymer is a pharmacologically active agent and present in an effective amount.

In a further aspect, the device of the disclosed method further comprises a pH-responsive material covering the core.

2. Preventing Fertility

In one aspect, the invention relates to methods of preventing fertility in a subject, the method comprising contacting a controlled-release device with mucous membrane tissue, wherein the device comprises: a) a core comprising: i) a water-swellable gel-forming polymer; ii) an effective amount of a pharmacologically active agent; and iii) optionally, an osmotic agent; b) a substantially inelastic, water-insoluble coating substantially enclosing the core, wherein at least a portion of the coating comprises a semipermeable membrane; and c) at least one orifice in the coating, wherein the orifice is positioned and dimensioned to allow controlled-release of the polymer from the core in response to swelling of the polymer, wherein the core is comprises no more than 15 wt % of the osmotic agent; and wherein the pharmacologically active agent is a birth control agent. Examples of birth control agents include, but are not limited to, ethinyl estradiol, norethindrone, levonorgestrel, ethynodiol diacetate, RU486, N9, mifepristone, mifegyne, mifeprex, 17a-ethinyl-levongestrel, 17b-hydroxy-estra-4,9,11-trien-3-one, estradiol, medroxyprogesterone acetate, nestorone, norgestrienone, progesterone, etonogestril (3-keto-desogestrel), progestin, megestrol, etono-progestin alonegestrel, and 17-acetoxy-16-methylene-19-norprogesterone.

In a further aspect, the tissue is selected from oral, nasal, vaginal, and rectal.

In a further aspect, the core comprises no more than 15 wt % of the osmotic agent. In a still further aspect, the core comprises no more than 10 wt % of the osmotic agent. In yet a further aspect, the core comprises no more than 5 wt % of the osmotic agent. In an even further aspect, the core does not comprise an osmotic agent.

In one aspect, the invention relates to methods of preventing fertility in a subject, the method comprising contacting a controlled-release device with vaginal membrane tissue, wherein the device comprises: a) a core comprising: i) a water-swellable gel-forming polymer and/or an osmotic agent; and ii) an effective amount of a pharmacologically active agent; b) a substantially inelastic, water-insoluble coating substantially enclosing the core, wherein at least a portion of the coating comprises a semipermeable membrane; and c) at least one orifice in the coating, wherein the orifice is positioned and dimensioned to allow controlled-release of the pharmacologically active agent from the core, wherein the pharmacologically active agent is a birth control agent.

In a further aspect, the polymer is a pharmacologically active agent and present in an effective amount.

In a further aspect, the birth control agent contains a polymer. In a still further aspect, the polymer contains a boronic acid.

In a further aspect, the birth control agent is toxic to sperm. In a still further aspect, the birth control agent acts as a barrier to sperm. In yet a further aspect, the birth control agent is a spermicide. In an even further aspect, the birth control agent is a ferrous salt.

In a further aspect, the device of the disclosed method further comprises a pH-responsive material covering the core.

3. Promoting Fertility

In one aspect, the invention relates to methods of promoting fertility in a subject, the method comprising contacting a controlled-release device with mucous membrane tissue, wherein the device comprises: a) a core comprising: i) a water-swellable gel-forming polymer; ii) an effective amount of a pharmacologically active agent; and iii) optionally, an osmotic agent; b) a substantially inelastic, water-insoluble coating substantially enclosing the core, wherein at least a portion of the coating comprises a semipermeable membrane; and c) at least one orifice in the coating, wherein the orifice is positioned and dimensioned to allow controlled-release of the polymer from the core in response to swelling of the polymer, wherein the core is comprises no more than 15 wt % of the osmotic agent; and wherein the pharmacologically active agent is a fertility agent. Examples of fertility agents include, but are not limited to, clomiphene, human chorionic gonadatropin (HCG), Leuprolide acetate, and menotropins.

In a further aspect, the tissue is selected from oral, nasal, vaginal, and rectal.

In a further aspect, the core comprises no more than 15 wt % of the osmotic agent. In a still further aspect, the core comprises no more than 10 wt % of the osmotic agent. In yet a further aspect, the core comprises no more than 5 wt % of the osmotic agent. In an even further aspect, the core does not comprise an osmotic agent.

In one aspect, the invention relates to method of promoting fertility in a subject, the method comprising contacting a controlled-release device with vaginal membrane tissue, wherein the device comprises: a) a core comprising: i) a water-swellable gel-forming polymer and/or an osmotic agent; and ii) an effective amount of a pharmacologically active agent; b) a substantially inelastic, water-insoluble coating substantially enclosing the core, wherein at least a portion of the coating comprises a semipermeable membrane; and c) at least one orifice in the coating, wherein the orifice is positioned and dimensioned to allow controlled-release of the pharmacologically active agent from the core, wherein the pharmacologically active agent is a birth control agent.

In a further aspect, the polymer is a pharmacologically active agent and present in an effective amount.

In a further aspect, the device of the disclosed method further comprises a pH-responsive material covering the core.

4. Prevention of a Sexually Transmitted Disease

In one aspect, the invention relates to methods of preventing a sexually transmitted disease in a subject, the method comprising contacting a controlled-release device with mucous membrane tissue, wherein the device comprises: a) a core comprising: i) a water-swellable gel-forming polymer; and ii) an effective amount of a pharmacologically active agent; and iii) optionally, an osmotic agent; b) a substantially inelastic, water-insoluble coating substantially enclosing the core, wherein at least a portion of the coating comprises a semipermeable membrane; and c) at least one orifice in the coating, wherein the orifice is positioned and dimensioned to allow controlled-release of the polymer from the core in response to swelling of the polymer, wherein the core comprises no more than 15 wt % of the osmotic agent; and wherein the pharmacologically active agent prevents the sexually transmitted disease. Examples of sexually transmitted diseases include, but are not limited to, HIV-1, HIV-2, AIDS, gonorrhea, chlamydia, trichomonal infections, human papilloma virus (HPV), herpes simplex virus (HSV), hepatitis B virus (HBV), syphilis, and genital herpes. In a further aspect, the sexually transmitted disease is HIV-1.

In a further aspect, the tissue is selected from oral, nasal, vaginal, and rectal.

In a further aspect, the core comprises no more than 15 wt % of the osmotic agent. In a still further aspect, the core comprises no more than 10 wt % of the osmotic agent. In yet a further aspect, the core comprises no more than 5 wt % of the osmotic agent. In an even further aspect, the core does not comprise an osmotic agent.

In one aspect, the invention relates to methods of preventing a sexually transmitted disease in a subject, the method comprising contacting a controlled-release device with vaginal membrane tissue, wherein the device comprises: a) a core comprising: i) a water-swellable gel-forming polymer and/or an osmotic agent; and ii) an effective amount of a pharmacologically active agent; b) a substantially inelastic, water-insoluble coating substantially enclosing the core, wherein at least a portion of the coating comprises a semipermeable membrane; and c) at least one orifice in the coating, wherein the orifice is positioned and dimensioned to allow controlled-release of the pharmacologically active agent from the core, wherein the pharmacologically active agent prevents the sexually transmitted disease.

In various aspects, the pharmacologically active agent is selected from entry inhibitors, fusion inhibitors, non-nucleoside reverse transcriptase inhibitors (NNRTIs), nucleoside reverse transcriptase inhibitors (NRTIs), nucleotide reverse transcriptase inhibitors, protease inhibitors, NCP7 inhibitors, detergents, surfactants, spermicides, inhibitors of viral adsorption, inhibitors of viral proteases, antivirals, antibiotics, antifungals, anti-inflammatories, antiparasitics, chemotherapeutics, antitoxins, immunotherapeutics, and integrase inhibitors.

In various aspects, the pharmacologically active agent is an anti-HIV agent. In a further aspect, the anti-HIV agent is selected from entry inhibitors, fusion inhibitors, non-nucleoside reverse transcriptase inhibitors (NNRTIs), nucleoside reverse transcriptase inhibitors (NRTIs), nucleotide reverse transcriptase inhibitors, NCP7 inhibitors, protease inhibitors, and integrase inhibitors. In a still further aspect, the entry inhibitor and/or fusion inhibitor is selected from Enfuvirtide (Fuzeon, T-20), AMD11070, PRO542, SCH-C, T-1249, TNX-355, cyanovirin, and maraviroc. In yet a further aspect, the non-nucleoside reverse transcriptase inhibitor is selected from delavirdine (Rescriptor), efavirenz (Sustiva), nevirapine (Viramune), calanolide A, capravirine, epivir, TMC125, adefovir, etravirine, rilpivirine, dapivirine, and lersivirine, and mixtures thereof. In an even further aspect, the nucleoside reverse transcriptase inhibitor and/or nucleotide reverse transcriptase inhibitor is selected from abacavir (Ziagen), didanosine (Videx, ddl), emtricitabine (Emtriva, FTC), lamivudine (Epivir, eTC), stavudine (Zerit, d4t), tenofovir (({[(2R)-1-(6-amino-9H-purin-9-yl)propan-2-yl]oxyl}methyl)phosphonic acid), tenofovir disoproxil fumarate, tenofovir alafenamide fumarate, zalcitabine (Hivid, ddc), zidovudine (Retrovir, AZT, ZDR), entecavir, and apricitabine, and mixtures thereof. In a still further aspect, the protease inhibitor is selected from amprenavir (Agenerase), atazanavir (Reyataz), fosamprenavir (Lexiva, 908), indinavir (Crixivan), nelfinavir (Viracept), ritonavir (Norvir), emtriva, saquinavir (Fortovase, Invirase), invirase, agenerase, lopinavir, tipranavir, and darunavir, and mixtures thereof. In yet a further aspect, the integrase inhibitor is selected from elvitegravir, raltegravir, GSK-572, and MK-2048, and mixtures thereof. Other anti-HIV agents include, for example, AMD-3100, BMS-806, BMS-793, C31G, carrageenan, zinc salts, CD4-IgG2, cellulose acetate phthalate, cellulose sulphate, cyclodextrins, dextrin-2-sulphate, mAb 2G12, mAb b12, Merck 167, plant lectins, poly naphthalene sulfate, poly sulfo-styrene, PRO2000, PSC-Rantes, SCH-C, SCH-D, T-20, TMC-125, UC-781, UK-427, UK-857, and Viramune, and mixtures thereof.

In various aspects, the pharmacologically active agent is an anti-HSV agent. In a further aspect, the anti-HSV agent is selected from acyclovir, ganciclovir, valacyclovir, famciclovir, penciclovir, imiquimod, resiquimod, vidarabine, brivudin, cidofovir, and foscarnet, erythromycin, azithromycin, clarithromycin, telithromycin, penicillin, cephalosporin, carbapenem, imipenem, meropenem, penicillin G, penicillin V, methicillin, oxacillin, cloxacillin, dicloxacillin, nafcillin, ampicillin, amoxicillin, carbenicillin, ticarcillin, meziocillin, piperacillin, azlocillin, temocillin, cepalothin, cephapirin, cephradine, cephaloridine, cefazolin, cefamandole, cefuroxime, cephalexin, cefprozil, cefaclor, loracarbef, cefoxitin, cefmetazole, cefotaxime, ceftizoxime, ceftriaxone, cefoperazone, ceftazidime, cefixime, cefpodoxime, ceftibuten, cefdinir, cefpirome, cefepime, astreonam, gentamycin, chloroquine, cetyl pyridinium chloride, nalidixic acid, oxolinic acid, norfloxacin, pefloxacin, enoxacin, ofloxacin, levofloxacin, ciprofloxacin, temafloxacin, lomefloxacin, fleroxacin, grepafloxacin, sparfloxacin, trovafloxacin, clinafloxacin, gatifloxacin, moxifloxacin, sitafloxacin, ganefloxacin, gemifloxacin, pazufloxacin, para-aminobenzoic acid, sulfadiazine, sulfisoxazole, sulfamethoxazole, sulfathalidine, streptomycin, neomycin, kanamycin, paromycin, gentamicin, tobramycin, amikacin, netilmicin, spectinomycin, sisomicin, dibekalin, sepamicin, tetracycline, chlortetracycline, demeclocycline, minocycline, oxytetracycline, methacycline, doxycycline, rifampicin (also called rifampin), rifapentine, rifabutin, bezoxazinorifamycin, rifaximin, lincomycin, clindamycin, vancomycin, teicoplanin, quinupristin, daflopristin, linezolid, polymyxin, colistin, colymycin, trimethoprim, bacitracin, triclosan, ascorbyl stearate, oleoyl sarcosine, dioctyl sulfosuccinate, vidarabine, phosphonomycin, an azole, polyene, echinocandin, pradimicin, fluconazole, isavuconazole, itraconazole, ketoconazole, miconazole, clortrimazole, voriconazole, posaconazole, rovuconazole, natamycin, lucensomycin, nystatin, amphotericin B, CANCIDAS®, beanomicins, nikkomycins, sordarins, allylamines, triclosan, piroctone, fenpropimorph, boric acid or borax, metronidazole, terconazole, tinidazole, clindamycin, and terbinafine, and mixtures thereof.

In a further aspect, the polymer is a pharmacologically active agent and present in an effective amount.

In a further aspect, the effective amount is a prophylactically effective amount.

In a further aspect, the device of the disclosed method further comprises a pH-responsive material covering the core.

5. Treatment of a Sexually Transmitted Disease

In one aspect, the invention relates to methods of treating a sexually transmitted disease in a subject, the method comprising contacting a controlled-release device with mucous membrane tissue, wherein the device comprises: a) a core comprising: i) a water-swellable gel-forming polymer; and ii) an effective amount of a pharmacologically active agent; and iii) optionally, an osmotic agent; b) a substantially inelastic, water-insoluble coating substantially enclosing the core, wherein at least a portion of the coating comprises a semipermeable membrane; and c) at least one orifice in the coating, wherein the orifice is positioned and dimensioned to allow controlled-release of the polymer from the core in response to swelling of the polymer, wherein the core comprises no more than 15 wt % of the osmotic agent; and wherein the pharmacologically active agent treats the sexually transmitted disease. Examples of sexually transmitted diseases include, but are not limited to, HIV-1, HIV-2, AIDS, gonorrhea, chlamydia, trichomonal infections, human papilloma virus (HPV), herpes simplex virus (HSV), hepatitis B virus (HBV), syphilis, and genital herpes. In a further aspect, the sexually transmitted disease is HIV-1.

In a further aspect, the tissue is selected from oral, nasal, vaginal, and rectal.

In a further aspect, the core comprises no more than 15 wt % of the osmotic agent. In a still further aspect, the core comprises no more than 10 wt % of the osmotic agent. In yet a further aspect, the core comprises no more than 5 wt % of the osmotic agent. In an even further aspect, the core does not comprise an osmotic agent.

In one aspect, the invention relates to methods of treating a sexually transmitted disease in a subject, the method comprising contacting a controlled-release device with vaginal membrane tissue, wherein the device comprises: a) a core comprising: i) a water-swellable gel-forming polymer and/or an osmotic agent; and ii) an effective amount of a pharmacologically active agent; b) a substantially inelastic, water-insoluble coating substantially enclosing the core, wherein at least a portion of the coating comprises a semipermeable membrane; and c) at least one orifice in the coating, wherein the orifice is positioned and dimensioned to allow controlled-release of the pharmacologically active agent from the core, wherein the pharmacologically active agent treats the sexually transmitted disease.

In various aspects, the pharmacologically active agent is selected from entry inhibitors, fusion inhibitors, non-nucleoside reverse transcriptase inhibitors (NNRTIs), nucleoside reverse transcriptase inhibitors (NRTIs), nucleotide reverse transcriptase inhibitors, protease inhibitors, NCP7 inhibitors, detergents, surfactants, spermicides, inhibitors of viral adsorption, inhibitors of viral proteases, antivirals, antibiotics, antifungals, anti-inflammatories, antiparasitics, chemotherapeutics, antitoxins, immunotherapeutics, and integrase inhibitors.

In various aspects, the pharmacologically active agent is an anti-HIV agent. In a further aspect, the anti-HIV agent is selected from entry inhibitors, fusion inhibitors, non-nucleoside reverse transcriptase inhibitors (NNRTIs), nucleoside reverse transcriptase inhibitors (NRTIs), nucleotide reverse transcriptase inhibitors, NCP7 inhibitors, protease inhibitors, and integrase inhibitors. In a still further aspect, the entry inhibitor and/or fusion inhibitor is selected from Enfuvirtide (Fuzeon, T-20), AMD11070, PRO542, SCH-C, T-1249, TNX-355, cyanovirin, and maraviroc. In yet a further aspect, the non-nucleoside reverse transcriptase inhibitor is selected from delavirdine (Rescriptor), efavirenz (Sustiva), nevirapine (Viramune), calanolide A, capravirine, epivir, TMC125, adefovir, etravirine, rilpivirine, dapivirine, and lersivirine, and mixtures thereof. In an even further aspect, the nucleoside reverse transcriptase inhibitor and/or nucleotide reverse transcriptase inhibitor is selected from abacavir (Ziagen), didanosine (Videx, ddl), emtricitabine (Emtriva, FTC), lamivudine (Epivir, eTC), stavudine (Zerit, d4t), tenofovir (({[(2R)-1-(6-amino-9H-purin-9-yl)propan-2-yl]oxy}methyl)phosphonic acid), tenofovir disoproxil fumarate, tenofovir alafenamide fumarate, zalcitabine (Hivid, ddc), zidovudine (Retrovir, AZT, ZDR), entecavir, and apricitabine, and mixtures thereof. In a still further aspect, the protease inhibitor is selected from amprenavir (Agenerase), atazanavir (Reyataz), fosamprenavir (Lexiva, 908), indinavir (Crixivan), nelfinavir (Viracept), ritonavir (Norvir), emtriva, saquinavir (Fortovase, Invirase), invirase, agenerase, lopinavir, tipranavir, and darunavir, and mixtures thereof. In yet a further aspect, the integrase inhibitor is selected from elvitegravir, raltegravir, GSK-572, and MK-2048, and mixtures thereof. Other anti-HIV agents include, for example, AMD-3100, BMS-806, BMS-793, C31G, carrageenan, zinc salts, CD4-IgG2, cellulose acetate phthalate, cellulose sulphate, cyclodextrins, dextrin-2-sulphate, mAb 2G12, mAb b12, Merck 167, plant lectins, poly naphthalene sulfate, poly sulfo-styrene, PRO2000, PSC-Rantes, SCH-C, SCH-D, T-20, TMC-125, UC-781, UK-427, UK-857, and Viramune, and mixtures thereof.

In various aspects, the pharmacologically active agent is an anti-HSV agent. In a further aspect, the anti-HSV agent is selected from acyclovir, ganciclovir, valacyclovir, famciclovir, penciclovir, imiquimod, resiquimod, vidarabine, brivudin, cidofovir, and foscarnet, and mixtures thereof.

In various aspects, the pharmacologically active agent is an anti-HPV agent. In a further aspect, the anti-HPV agent is selected from pyrrole polyamides, lopinavir, and carrageenan, zinc salts, erythromycin, azithromycin, clarithromycin, telithromycin, penicillin, cephalosporin, carbapenem, imipenem, meropenem, penicillin G, penicillin V, methicillin, oxacillin, cloxacillin, dicloxacillin, nafcillin, ampicillin, amoxicillin, carbenicillin, ticarcillin, meziocillin, piperacillin, azlocillin, temocillin, cepalothin, cephapirin, cephradine, cephaloridine, cefazolin, cefamandole, cefuroxime, cephalexin, cefprozil, cefaclor, loracarbef, cefoxitin, cefmetazole, cefotaxime, ceftizoxime, ceftriaxone, cefoperazone, ceftazidime, cefixime, cefpodoxime, ceftibuten, cefdinir, cefpirome, cefepime, astreonam, gentamycin, chloroquine, cetyl pyridinium chloride, nalidixic acid, oxolinic acid, norfloxacin, pefloxacin, enoxacin, ofloxacin, levofloxacin, ciprofloxacin, temafloxacin, lomefloxacin, fleroxacin, grepafloxacin, sparfloxacin, trovafloxacin, clinafloxacin, gatifloxacin, moxifloxacin, sitafloxacin, ganefloxacin, gemifloxacin, pazufloxacin, para-aminobenzoic acid, sulfadiazine, sulfisoxazole, sulfamethoxazole, sulfathalidine, streptomycin, neomycin, kanamycin, paromycin, gentamicin, tobramycin, amikacin, netilmicin, spectinomycin, sisomicin, dibekalin, sepamicin, tetracycline, chlortetracycline, demeclocycline, minocycline, oxytetracycline, methacycline, doxycycline, rifampicin (also called rifampin), rifapentine, rifabutin, bezoxazinorifamycin, rifaximin, lincomycin, clindamycin, vancomycin, teicoplanin, quinupristin, daflopristin, linezolid, polymyxin, colistin, colymycin, trimethoprim, bacitracin, triclosan, ascorbyl stearate, oleoyl sarcosine, dioctyl sulfosuccinate, vidarabine, and phosphonomycin, fluconazole, isavuconazole, itraconazole, ketoconazole, miconazole, clortrimazole, voriconazole, posaconazole, rovuconazole, natamycin, lucensomycin, nystatin, amphotericin B, CANCIDAS®, beanomicins, nikkomycins, sordarins, allylamines, triclosan, piroctone, fenpropimorph, boric acid or borax, metronidazole, terconazole, tinidazole, clindamycin, and terbinafine, and mixtures thereof.

In a further aspect, the polymer is a pharmacologically active agent and present in an effective amount.

In a further aspect, the effective amount is a pharmaceutically effective amount.

In a further aspect, the device of the disclosed method further comprises a pH-responsive material covering the core.

6. Hormone Replacement

In one aspect, the invention relates to methods for hormone replacement in a subject, the method comprising contacting a controlled-release device with mucous membrane tissue, wherein the device comprises: a) a core comprising: i) a water-swellable gel-forming polymer; ii) an effective amount of a pharmacologically active agent; and iii) optionally, an osmotic agent; b) a substantially inelastic, water-insoluble coating substantially enclosing the core, wherein at least a portion of the coating comprises a semipermeable membrane; and c) at least one orifice in the coating, wherein the orifice is positioned and dimensioned to allow controlled-release of the polymer from the core in response to swelling of the polymer, wherein the core is comprises no more than 15 wt % of the osmotic agent; and wherein the pharmacologically active agent is a hormone replacement agent. Thus, in various aspects, exemplary hormone replacement agents include gonadatropin releasing hormone agonists, leuprolide acetate, estrogen, progesterone, testosterone, follicle stimulating hormone (FSH), and progestin. In a further aspect, the hormone replacement agent is progestin.

In a further aspect, the tissue is selected from oral, nasal, vaginal, and rectal.

In a further aspect, the core comprises no more than 15 wt % of the osmotic agent. In a still further aspect, the core comprises no more than 10 wt % of the osmotic agent. In yet a further aspect, the core comprises no more than 5 wt % of the osmotic agent. In an even further aspect, the core does not comprise an osmotic agent.

In one aspect, the invention relates to method of hormone replacement in a subject, the method comprising contacting a controlled-release device with vaginal membrane tissue, wherein the device comprises: a) a core comprising: i) a water-swellable gel-forming polymer and/or an osmotic agent; and ii) an effective amount of a pharmacologically active agent; b) a substantially inelastic, water-insoluble coating substantially enclosing the core, wherein at least a portion of the coating comprises a semipermeable membrane; and c) at least one orifice in the coating, wherein the orifice is positioned and dimensioned to allow controlled-release of the pharmacologically active agent from the core, wherein the pharmacologically active agent is a hormone replacement agent.

In a further aspect, the polymer is a pharmacologically active agent and present in an effective amount.

In a further aspect, the device of the disclosed method further comprises a pH-responsive material covering the core.

7. Cervical Ripening

In one aspect, the invention relates to methods for cervical ripening in a subject, the method comprising contacting a controlled-release device with mucous membrane tissue, wherein the device comprises: a) a core comprising: i) a water-swellable gel-forming polymer; ii) an effective amount of a pharmacologically active agent; and iii) optionally, an osmotic agent; b) a substantially inelastic, water-insoluble coating substantially enclosing the core, wherein at least a portion of the coating comprises a semipermeable membrane; and c) at least one orifice in the coating, wherein the orifice is positioned and dimensioned to allow controlled-release of the polymer from the core in response to swelling of the polymer, wherein the core is comprises no more than 15 wt % of the osmotic agent; and wherein the pharmacologically active agent is a cervical ripening agent. Examples of cervical ripening agents include, but are not limited to, prostaglandin E2, cytotec, laminaria tents, misoprostol and dinoprostone.

In a further aspect, the tissue is selected from oral, nasal, vaginal, and rectal. In a still further aspect, the tissue is vaginal.

In a further aspect, the core comprises no more than 15 wt % of the osmotic agent. In a still further aspect, the core comprises no more than 10 wt % of the osmotic agent. In yet a further aspect, the core comprises no more than 5 wt % of the osmotic agent. In an even further aspect, the core does not comprise an osmotic agent.

In one aspect, the invention relates to method of cervical ripening in a subject, the method comprising contacting a controlled-release device with vaginal membrane tissue, wherein the device comprises: a) a core comprising: i) a water-swellable gel-forming polymer and/or an osmotic agent; and ii) an effective amount of a pharmacologically active agent; b) a substantially inelastic, water-insoluble coating substantially enclosing the core, wherein at least a portion of the coating comprises a semipermeable membrane; and c) at least one orifice in the coating, wherein the orifice is positioned and dimensioned to allow controlled-release of the pharmacologically active agent from the core, wherein the pharmacologically active agent is a cervical ripening agent.

In a further aspect, the polymer is a pharmacologically active agent and present in an effective amount.

In a further aspect, the device of the disclosed method further comprises a pH-responsive material covering the core. In a still further aspect, the device of the disclosed method further comprises a pH-responsive material covering the core and the tissue is vaginal. In yet a further aspect, the method further comprises flushing the vaginal membrane tissue with a basic solution. In an even further aspect, the basic solution is phosphate buffered saline. In a still further aspect, the basic solution is phosphate buffered saline pH 7.4.

8. Treatment of a Mucosal Infection

In one aspect, the invention relates to methods of treating mucosal infection in a subject, the method comprising contacting a controlled-release device with mucous membrane tissue, wherein the device comprises: a) a core comprising: i) a water-swellable gel-forming polymer; ii) an effective amount of a pharmacologically active agent; and iii) optionally, an osmotic agent; b) a substantially inelastic, water-insoluble coating substantially enclosing the core, wherein at least a portion of the coating comprises a semipermeable membrane; and c) at least one orifice in the coating, wherein the orifice is positioned and dimensioned to allow controlled-release of the polymer from the core in response to swelling of the polymer, wherein the core is comprises no more than 15 wt % of the osmotic agent; and wherein the pharmacologically active agent is an agent that treats a mucosal infection. In a further aspect, the agent that treats a mucosal infection is selected from an antiviral, antibacterial, and/or antifungal agent. In a further aspect, the antiviral, antibacterial, and/or antifungal agent is an agent that treats a mucosal infection. Examples of mucosal infections include, but are not limited to bacterial vaginosis, yeast infections, vaginosis induced by Candida infections, and vulvovaginal candidiasis.

In a further aspect, the tissue is selected from oral, nasal, vaginal, and rectal.

In a further aspect, the core comprises no more than 15 wt % of the osmotic agent. In a still further aspect, the core comprises no more than 10 wt % of the osmotic agent. In yet a further aspect, the core comprises no more than 5 wt % of the osmotic agent. In an even further aspect, the core does not comprise an osmotic agent.

In one aspect, the invention relates to method of treating a mucosal infection in a subject, the method comprising contacting a controlled-release device with vaginal membrane tissue, wherein the device comprises: a) a core comprising: i) a water-swellable gel-forming polymer and/or an osmotic agent; and ii) an effective amount of a pharmacologically active agent; b) a substantially inelastic, water-insoluble coating substantially enclosing the core, wherein at least a portion of the coating comprises a semipermeable membrane; and c) at least one orifice in the coating, wherein the orifice is positioned and dimensioned to allow controlled-release of the pharmacologically active agent from the core, wherein the pharmacologically active agent is an antiviral, antibacterial, and/or antifungal agent. In a further aspect, the antiviral, antibacterial, and/or antifungal agent is an agent that treats a mucosal infection.

In a further aspect, the agent that treats a mucosal infection is selected from boric acid or borax, metronidazole, clotrimazole, miconazole, terconazole, tinidazole, ‘and clindamycin.

Examples of antiviral agents include, but are not limited to, acemannan, acyclovir, acyclovir sodium, adamantanamine, adefovir, adenine arabinoside, alovudine, alvircept sudotox, amantadine hydrochloride, aranotin, arildone, atevirdine mesylate, avridine, cidofovir, cipamfylline, cytarabine hydrochloride, BMS 806, C31G, carrageenan, zinc salts, cellulose sulfate, cyclodextrins, dapivirine, delavirdine mesylate, desciclovir, dextrin 2-sulfate, didanosine, disoxaril, dolutegravir, edoxudine, enviradene, envirozime, etravirine, famciclovir, famotine hydrochloride, fiacitabine, fialuridine, fosarilate, foscarnet sodium, fosfonet sodium, FTC, ganciclovir, ganciclovir sodium, GSK 1265744, 9-2-hydroxy-ethoxy methylguanine, ibalizumab, idoxuridine, interferon, 5-iodo-2′-deoxyuridine, IQP-0528, kethoxal, lamivudine, lobucavir, maraviroc, memotine pirodavir, penciclovir, raltegravir, ribavirin, rimantadine hydrochloride, rilpivirine (TMC-278), saquinavir mesylate, SCH-C, SCH-D, somantadine hydrochloride, sorivudine, statolon, stavudine, T20, tilorone hydrochloride, TMC120, TMC125, trifluridine, trifluorothymidine, tenofovir, tenofovir alefenamide, tenofovir disoproxyl fumarate, prodrugs of tenofovir, UC-781, UK-427, UK-857, valacyclovir, valacyclovir hydrochloride, vidarabine, vidarabine phosphate, vidarabine sodium phosphate, viroxime, zalcitabene, zidovudine, and zinviroxime.

Examples of antibacterial agents include, but are not limited to, erythromycin, azithromycin, clarithromycin, telithromycin, penicillin, cephalosporin, carbapenem, imipenem, meropenem, penicillin G, penicillin V, methicillin, oxacillin, cloxacillin, dicloxacillin, nafcillin, ampicillin, amoxicillin, carbenicillin, ticarcillin, meziocillin, piperacillin, azlocillin, temocillin, cepalothin, cephapirin, cephradine, cephaloridine, cefazolin, cefamandole, cefuroxime, cephalexin, cefprozil, cefaclor, loracarbef, cefoxitin, cefmetazole, cefotaxime, ceftizoxime, ceftriaxone, cefoperazone, ceftazidime, cefixime, cefpodoxime, ceftibuten, cefdinir, cefpirome, cefepime, astreonam, gentamycin, chloroquine, cetyl pyridinium chloride, nalidixic acid, oxolinic acid, norfloxacin, pefloxacin, enoxacin, ofloxacin, levofloxacin, ciprofloxacin, temafloxacin, lomefloxacin, fleroxacin, grepafloxacin, sparfloxacin, trovafloxacin, clinafloxacin, gatifloxacin, moxifloxacin, sitafloxacin, ganefloxacin, gemifloxacin, pazufloxacin, para-aminobenzoic acid, sulfadiazine, sulfisoxazole, sulfamethoxazole, sulfathalidine, streptomycin, neomycin, kanamycin, paromycin, gentamicin, tobramycin, amikacin, netilmicin, spectinomycin, sisomicin, dibekalin, sepamicin, tetracycline, chlortetracycline, demeclocycline, minocycline, oxytetracycline, methacycline, doxycycline, rifampicin (also called rifampin), rifapentine, rifabutin, bezoxazinorifamycin, rifaximin, lincomycin, clindamycin, vancomycin, teicoplanin, quinupristin, daflopristin, linezolid, polymyxin, colistin, colymycin, trimethoprim, bacitracin, triclosan, ascorbyl stearate, oleoyl sarcosine, dioctyl sulfosuccinate, vidarabine, and phosphonomycin.

In various aspects, the antifungal agent is selected from an azole, polyene, echinocandin, and pradimicin. In a further aspect, the antifungal agent is selected from fluconazole, isavuconazole, itraconazole, ketoconazole, miconazole, clortrimazole, voriconazole, posaconazole, rovuconazole, natamycin, lucensomycin, nystatin, amphotericin B, CANCIDAS®, beanomicins, nikkomycins, sordarins, allylamines, triclosan, piroctone, fenpropimorph, boric acid or borax, metronidazole, terconazole, tinidazole, and clindamycin, and terbinafine.

In a further aspect, the polymer is a pharmacologically active agent and present in an effective amount.

In a further aspect, the device of the disclosed method further comprises a pH-responsive material covering the core.

9. Delivering Biologics

In one aspect, the invention relates to methods of delivering biologics, the method comprising contacting a controlled-release device with mucous membrane tissue, wherein the device comprises: a) a core comprising: i) a water-swellable gel-forming polymer; ii) an effective amount of a pharmacologically active agent; and iii) optionally, an osmotic agent; b) a substantially inelastic, water-insoluble coating substantially enclosing the core, wherein at least a portion of the coating comprises a semipermeable membrane; and c) at least one orifice in the coating, wherein the orifice is positioned and dimensioned to allow controlled-release of the polymer from the core in response to swelling of the polymer, wherein the core is comprises no more than 15 wt % of the osmotic agent; and wherein the pharmacologically active agent is a biologic.

In a further aspect, the tissue is selected from oral, nasal, vaginal, and rectal.

In a further aspect, the core comprises no more than 15 wt % of the osmotic agent. In a still further aspect, the core comprises no more than 10 wt % of the osmotic agent. In yet a further aspect, the core comprises no more than 5 wt % of the osmotic agent. In an even further aspect, the core does not comprise an osmotic agent.

In one aspect, the invention relates to method of delivering biologics, the method comprising contacting a controlled-release device with vaginal membrane tissue, wherein the device comprises: a) a core comprising: i) a water-swellable gel-forming polymer and/or an osmotic agent; and ii) an effective amount of a pharmacologically active agent; b) a substantially inelastic, water-insoluble coating substantially enclosing the core, wherein at least a portion of the coating comprises a semipermeable membrane; and c) at least one orifice in the coating, wherein the orifice is positioned and dimensioned to allow controlled-release of the pharmacologically active agent from the core, wherein the pharmacologically active agent is a biologic.

In a further aspect, the biologic is a freeze dried organism.

In a further aspect, the biologic is a probiotic. In a still further aspect, the biologic is a probiotic. In yet a further aspect, the probiotic is selected from Lactobacillus bulgaricus, Lactobacillus casei subsp. Rhamnosus, Lactobacillus casei subsp. Casei, Lactobacillus salivarius, Lactobacillus brevis, Lactobacillus reuteri, Lactococcus lactis subsp. Lactis, Enterococcus faecium, Lactobacillus plantarum, Streptococcus thermophilus, Bifidobacterium infantis, Bifidobacterium bifidum, Bifidobacterium longum, Saccharomyces boulardii, Lactobacillus acidophilus, whey proteins, lysozyme, lactoferrin, lactoperoxidase, xanthine oxidase, vitamin-binding proteins and immunoglobulins, mannose, oligosaccharides, starches, mannan oligosaccharides, trans-galacto-oligosaccharide, inulin, and fructo-oligosaccharide.

In a further aspect, the polymer is a pharmacologically active agent and present in an effective amount.

In a further aspect, the device of the disclosed method further comprises a pH-responsive material covering the core.

10. Vaginal Dryness

In one aspect, the invention relates to methods of treating vaginal dryness in a subject, the method comprising contacting a controlled-release device with mucous membrane tissue, wherein the device comprises: a) a core comprising: i) a water-swellable gel-forming polymer; ii) an effective amount of a pharmacologically active agent; and iii) optionally, an osmotic agent; b) a substantially inelastic, water-insoluble coating substantially enclosing the core, wherein at least a portion of the coating comprises a semipermeable membrane; and c) at least one orifice in the coating, wherein the orifice is positioned and dimensioned to allow controlled-release of the polymer from the core in response to swelling of the polymer, wherein the core is comprises no more than 15 wt % of the osmotic agent; and wherein the pharmacologically active agent is a lubricant. Examples of lubricants include, but are not limited to, high molecular weight polyethylene glycols and glycol esters, triglycerides, glycerin, polycarbophil and polysorbates, pyridine, squalene, urea, complex alcohols, aldehydes, ketones, stearic acid, stearate, isopropyl palmitate, petrolatum, aloe barbadensis (Aloe Vera) leaf juice, cucumus sativus extract, helianthus annulus seed oil, soybean sterol, vitamin E acetate, vitamin A palmitate, provitamin B5, sodium acrylate/acryloyldimethyl taurate copolymer, dimethicone, glyceryl stearate, ceylalcohol, lecithin, mineral water, sodium PCA, potassium lactate, collagen, aminoacids, triethanolamine, DMDM hydantoin, iodopropynyl, butylcarbamate, disodium EDTA, and titanium dioxide.

In a further aspect, the tissue is selected from oral, nasal, vaginal, and rectal.

In a further aspect, the core comprises no more than 15 wt % of the osmotic agent. In a still further aspect, the core comprises no more than 10 wt % of the osmotic agent. In yet a further aspect, the core comprises no more than 5 wt % of the osmotic agent. In an even further aspect, the core does not comprise an osmotic agent.

In one aspect, the invention relates to method of treating vaginal dryness in a subject, the method comprising contacting a controlled-release device with vaginal membrane tissue, wherein the device comprises: a) a core comprising: i) a water-swellable gel-forming polymer and/or an osmotic agent; and ii) an effective amount of a pharmacologically active agent; b) a substantially inelastic, water-insoluble coating substantially enclosing the core, wherein at least a portion of the coating comprises a semipermeable membrane; and c) at least one orifice in the coating, wherein the orifice is positioned and dimensioned to allow controlled-release of the pharmacologically active agent from the core, wherein the pharmacologically active agent is a lubricant.

In a further aspect, the polymer is a pharmacologically active agent and present in an effective amount.

In a further aspect, the device of the disclosed method further comprises a pH-responsive material covering the core.

11. Treating Uterine Fibroids

In one aspect, the invention relates to methods of treating uterine fibroids in a subject, the method comprising contacting a controlled-release device with mucous membrane tissue, wherein the device comprises: a) a core comprising: i) a water-swellable gel-forming polymer; ii) an effective amount of a pharmacologically active agent; and iii) optionally, an osmotic agent; b) a substantially inelastic, water-insoluble coating substantially enclosing the core, wherein at least a portion of the coating comprises a semipermeable membrane; and c) at least one orifice in the coating, wherein the orifice is positioned and dimensioned to allow controlled-release of the polymer from the core in response to swelling of the polymer, wherein the core is comprises no more than 15 wt % of the osmotic agent; and wherein the pharmacologically active agent is an agent that treats uterine fibroids.

In a further aspect, the tissue is selected from oral, nasal, vaginal, and rectal.

In a further aspect, the core comprises no more than 15 wt % of the osmotic agent. In a still further aspect, the core comprises no more than 10 wt % of the osmotic agent. In yet a further aspect, the core comprises no more than 5 wt % of the osmotic agent. In an even further aspect, the core does not comprise an osmotic agent.

In one aspect, the invention relates to method of treating uterine fibroids in a subject, the method comprising contacting a controlled-release device with vaginal membrane tissue, wherein the device comprises: a) a core comprising: i) a water-swellable gel-forming polymer and/or an osmotic agent; and ii) an effective amount of a pharmacologically active agent; b) a substantially inelastic, water-insoluble coating substantially enclosing the core, wherein at least a portion of the coating comprises a semipermeable membrane; and c) at least one orifice in the coating, wherein the orifice is positioned and dimensioned to allow controlled-release of the pharmacologically active agent from the core, wherein the pharmacologically active agent is an agent that treats uterine fibroids.

In a further aspect, the agent that treats uterine fibroids binds to the GnRH receptor. In a still further aspect, the agent that treats uterine fibroids is a GnRH agonist. In yet a further aspect, the GnRH agonist is selected from leuprolide, dislorelin, triptorelin, leoprorelin, buserelin, nafarelin, goserelin, [Dlys6]GnRH, [Dala]GnRH, avorelin, histerelin, PTL 03301, AN 207, TX 397, AN 201, and SPD 424.

In a further aspect, the polymer is a pharmacologically active agent and present in an effective amount.

In a further aspect, the device of the disclosed method further comprises a pH-responsive material covering the core.

12. Treating Reproductive Cancers

In one aspect, the invention relates to methods of treating reproductive cancers, the method comprising contacting a controlled-release device with mucous membrane tissue, wherein the device comprises: a) a core comprising: i) a water-swellable gel-forming polymer; ii) an effective amount of a pharmacologically active agent; and iii) optionally, an osmotic agent; b) a substantially inelastic, water-insoluble coating substantially enclosing the core, wherein at least a portion of the coating comprises a semipermeable membrane; and c) at least one orifice in the coating, wherein the orifice is positioned and dimensioned to allow controlled-release of the polymer from the core in response to swelling of the polymer, wherein the core is comprises no more than 15 wt % of the osmotic agent; and wherein the pharmacologically active agent is an anti-cancer agent. Examples of anti-cancer agents include, but are not limited to, fluorouracil, cisplatin, doxorubicin, leuprolide acetate, and paclitaxel.

In a further aspect, the tissue is selected from oral, nasal, vaginal, and rectal.

In a further aspect, the core comprises no more than 15 wt % of the osmotic agent. In a still further aspect, the core comprises no more than 10 wt % of the osmotic agent. In yet a further aspect, the core comprises no more than 5 wt % of the osmotic agent. In an even further aspect, the core does not comprise an osmotic agent.

In one aspect, the invention relates to method of treating reproductive cancers in a subject, the method comprising contacting a controlled-release device with vaginal membrane tissue, wherein the device comprises: a) a core comprising: i) a water-swellable gel-forming polymer and/or an osmotic agent; and ii) an effective amount of a pharmacologically active agent; b) a substantially inelastic, water-insoluble coating substantially enclosing the core, wherein at least a portion of the coating comprises a semipermeable membrane; and c) at least one orifice in the coating, wherein the orifice is positioned and dimensioned to allow controlled-release of the pharmacologically active agent from the core, wherein the pharmacologically active agent is an anti-cancer agent.

In a further aspect, the anti-cancer agent binds to the GnRH receptor. In a still further aspect, the anti-cancer agent is a GnRH agonist. In yet a further aspect, the GnRH agonist is selected from leuprolide, dislorelin, triptorelin, leoprorelin, buserelin, nafarelin, goserelin, [Dlys6]GnRH, [Dala]GnRH, avorelin, histerelin, PTL 03301, AN 207, TX 397, AN 201, and SPD 424.

In a further aspect, the polymer is a pharmacologically active agent and present in an effective amount.

In a further aspect, the device of the disclosed method further comprises a pH-responsive material covering the core.

13. Treating Nausea Gravidarum

In one aspect, the invention relates to methods of treating nausea gravidarum, the method comprising contacting a controlled-release device with mucous membrane tissue, wherein the device comprises: a) a core comprising: i) a water-swellable gel-forming polymer; ii) an effective amount of a pharmacologically active agent; and iii) optionally, an osmotic agent; b) a substantially inelastic, water-insoluble coating substantially enclosing the core, wherein at least a portion of the coating comprises a semipermeable membrane; and c) at least one orifice in the coating, wherein the orifice is positioned and dimensioned to allow controlled-release of the polymer from the core in response to swelling of the polymer, wherein the core is comprises no more than 15 wt % of the osmotic agent; and wherein the pharmacologically active agent is an antiemetic. Examples of antiemetics include, but are not limited to, bromocriptine, diphenhydramine, hydroxyzine, meclizine, metoclopramide, haloperidol, droperidol, lorazepam, alprazolam, dronabinol, dexamethasone, methylprednisolone, prochlorperazine, promethazine, chlorpromazine, ondansetron, dolasetron, granisetron, and aprepitant.

In a further aspect, the tissue is selected from oral, nasal, vaginal, and rectal.

In a further aspect, the core comprises no more than 15 wt % of the osmotic agent. In a still further aspect, the core comprises no more than 10 wt % of the osmotic agent. In yet a further aspect, the core comprises no more than 5 wt % of the osmotic agent. In an even further aspect, the core does not comprise an osmotic agent.

In one aspect, the invention relates to method of treating nausea gravidarum in a subject, the method comprising contacting a controlled-release device with vaginal membrane tissue, wherein the device comprises: a) a core comprising: i) a water-swellable gel-forming polymer and/or an osmotic agent; and ii) an effective amount of a pharmacologically active agent; b) a substantially inelastic, water-insoluble coating substantially enclosing the core, wherein at least a portion of the coating comprises a semipermeable membrane; and c) at least one orifice in the coating, wherein the orifice is positioned and dimensioned to allow controlled-release of the pharmacologically active agent from the core, wherein the pharmacologically active agent is an antiemetic.

In a further aspect, the antiemetic is selected from an antihistamine, benzamide, butyrophenone, benzodiazepine, cannabinoid, corticosteroid, phenothiazine, serotonin antagonist, and neurokinin-1-receptor antagonist.

In a further aspect, the polymer is a pharmacologically active agent and present in an effective amount.

In a further aspect, the device of the disclosed method further comprises a pH-responsive material covering the core.

14. Treating Endometriosis

In one aspect, the invention relates to methods of treating endometriosis, the method comprising contacting a controlled-release device with mucous membrane tissue, wherein the device comprises: a) a core comprising: i) a water-swellable gel-forming polymer; ii) an effective amount of a pharmacologically active agent; and iii) optionally, an osmotic agent; b) a substantially inelastic, water-insoluble coating substantially enclosing the core, wherein at least a portion of the coating comprises a semipermeable membrane; and c) at least one orifice in the coating, wherein the orifice is positioned and dimensioned to allow controlled-release of the polymer from the core in response to swelling of the polymer, wherein the core is comprises no more than 15 wt % of the osmotic agent; and wherein the pharmacologically active agent is an agent that treats endometriosis.

In a further aspect, the tissue is selected from oral, nasal, vaginal, and rectal.

In a further aspect, the core comprises no more than 15 wt % of the osmotic agent. In a still further aspect, the core comprises no more than 10 wt % of the osmotic agent. In yet a further aspect, the core comprises no more than 5 wt % of the osmotic agent. In an even further aspect, the core does not comprise an osmotic agent.

In one aspect, the invention relates to method of treating endometriosis in a subject, the method comprising contacting a controlled-release device with vaginal membrane tissue, wherein the device comprises: a) a core comprising: i) a water-swellable gel-forming polymer and/or an osmotic agent; and ii) an effective amount of a pharmacologically active agent; b) a substantially inelastic, water-insoluble coating substantially enclosing the core, wherein at least a portion of the coating comprises a semipermeable membrane; and c) at least one orifice in the coating, wherein the orifice is positioned and dimensioned to allow controlled-release of the pharmacologically active agent from the core, wherein the pharmacologically active agent is an agent that treats endometriosis.

In a further aspect, the agent that treats endometriosis is terbutaline, Abarelix, SERMs (Selective Estrogen Receptor Modulators), Extracellular Matrix Modulators, and RU-486.

In a further aspect, the agent that treats endometriosis binds to the GnRH receptor. In a still further aspect, the agent that treats endometriosis is a GnRH agonist. In yet a further aspect, the GnRH agonist is selected from leuprolide, dislorelin, triptorelin, leoprorelin, buserelin, nafarelin, goserelin, [Dlys6]GnRH, [Dala]GnRH, avorelin, histerelin, PTL 03301, AN 207, TX 397, AN 201, and SPD 424.

In a further aspect, the polymer is a pharmacologically active agent and present in an effective amount.

In a further aspect, the device of the disclosed method further comprises a pH-responsive material covering the core.

15. Manufacture of a Medicament

In one aspect, the invention relates to methods for the manufacture of a medicament for preventing fertility, promoting fertility, preventing a sexually transmitted disease, treating a sexually transmitted disease, hormone replacement, cervical ripening, treating a mucosal infection, delivering a biologic, treating vaginal dryness, treating uterine fibroids, treating reproductive cancers, treating nausea gravidarum, and treating endometriosis.

In a further aspect, the subject is a mammal. In a still further aspect, the subject is a human.

16. Non-Medical Uses

Also provided are non-medical uses of the disclosed devices and products as pharmacological tools in the development and standardization of in vitro and in vivo test systems for the evaluation of pharmacologically active agents in laboratory animals such as cats, dogs, rabbits, monkeys, rats, and mice, as part of the search for new therapeutic agents for preventing fertility, promoting fertility, preventing a sexually transmitted disease, treating a sexually transmitted disease, hormone replacement, cervical ripening, treating a mucosal infection, delivering a biologic, treating vaginal dryness, treating uterine fibroids, treating reproductive cancers, treating nausea gravidarum, and treating endometriosis.

It is contemplated that the disclosed uses can be employed in connection with the disclosed devices, products of disclosed methods of making, delivery methods, and/or the disclosed kits.

17. Kits

In one aspect, the invention relates to a kit comprising a disclosed device, wherein the pharmacologically active agent is present, and at least one of: a) a second pharmacologically active agent; b) a second controlled release device comprising a second pharmacologically active agent; c) an applicator; and d) instructions for contacting mucous membrane tissue of a mammal.

In a further aspect, the pharmacologically active agent is selected from a birth control agent, a fertility agent, an agent that treats a sexually transmitted disease, an agent that prevents a sexually transmitted disease, a hormone replacement agent, a cervical ripening agent, an agent that treats a mucosal infection, an agent that treats vaginal dryness, an agent that treats uterine fibroids, an agent that treats reproductive cancers, an agent that treats nausea gravidarum, an agent that treats endometriosis, and a biologic. In a still further aspect, the pharmacologically active agent is a birth control agent. In yet a further aspect, the pharmacologically active agent is a fertility agent. In an even further aspect, the pharmacologically active agent is a hormone replacement agent. In a still further aspect, the pharmacologically active agent is a cervical ripening agent. In yet a further aspect, the pharmacologically active agent treats a mucosal infection. In an even further aspect, the pharmacologically active agent treats vaginal dryness. In a still further aspect, the pharmacologically active agent treats uterine fibroids. In yet a further aspect, the pharmacologically active agent treats reproductive cancers. In an even further aspect, the pharmacologically active agent treats nausea gravidarum. In a still further aspect, the pharmacologically active agent treats endometriosis.

In a further aspect, the pharmacologically active agent treats a sexually transmitted disease. In a still further aspect, the pharmacologically active agent prevents a sexually transmitted disease. In yet a further aspect, the sexually transmitted disease is HIV-1.

In a further aspect, the pharmacologically active agent is a biologic. In a still further aspect, the biologic is a freeze dried organism. In yet a further aspect, the biologic is a probiotic.

The kits can also comprise compounds and/or products co-packaged, co-formulated, and/or co-delivered with other components. For example, a drug manufacturer, a drug reseller, a physician, a compounding shop, or a pharmacist can provide a kit comprising a disclosed compound and/or product and another component for delivery to a patient.

It is contemplated that the disclosed kits can be used in connection with the disclosed methods of making, the disclosed methods of using, and/or the disclosed compositions.

### F. PROPHETIC EXAMPLES OF USING A CONTROLLED-RELEASE DEVICE

1. Controlled Delivery of an Antiretroviral Agent for Chemoprophylaxis Against HIV-1 Infections

In one prophetic example, an osmotic pump is formulated for controlled delivery of an antiretroviral agent for up to 7 days for prevention against sexually transmitted HIV infections. In one aspect, the antiretroviral agent is a viral entry inhibitors. In a further aspect, the antiretroviral agent is a nucleotide analogue reverse transcriptase inhibitor, for example, tenofovir and its prodrugs, tenofovir disoproxil fumarate (TDF) and tenofovir alafenamide fumarate (TAF, GS-7340). The antiretroviral agent is blended with a gel-forming polymer and compressed into a pellet. In various aspects, two or more antiretroviral agents in differing amounts as necessary to achieve therapeutically active doses may be blended with gel-forming polymers to form a pellet. The membrane in this example has adequate water vapor transmission rate to allow quick hydration of the pellet. A continuous controlled therapeutically effective dose of the antiretroviral agent is delivered in the vaginal tract for 7 days. Upon release of contents, the membrane loses its integrity and breaks into small fragments that are eliminated from the vaginal tract.

2. Peri-Coital Delivery of an Antiretroviral Agent for Chemoprophylaxis Against HIV-1 Infection

In one prophetic example, an osmotic pump is formulated for controlled delivery of a microbicide for a maximum period of 7 days along with high doses in the presence of semen in the vaginal tract for prevention against sexually transmitted HIV infections. In one aspect, the antiretroviral agent is selected from the class of viral entry inhibitors. In a further aspect, the antiretroviral agent belongs to the class of nucleotide analogue reverse transcriptase inhibitors, for example, tenofovir and its prodrugs, tenofovir disoproxil fumarate (TDF) and tenofovir alafenamide fumarate (TAF, GS-7340). The antiretroviral agent is blended with a gel-forming polymer and compressed into a pellet. In one aspect, two or more antiretroviral agents in differing amounts as necessary to achieve therapeutically active doses may be blended with gel-forming polymers to form a pellet. The membrane in this example is pH sensitive and has adequate water vapor transmission rate to allow quick hydration of the pellet. A continuous controlled dose of the antiretroviral agent is delivered in the vaginal tract for a maximum period of 7 days. Upon release of contents, the membrane loses its integrity and breaks into small fragments that are eliminated from the vaginal tract. This membrane can instantaneously dissolve due to pH change upon introduction of semen in the vaginal tract releasing high neutralizing doses of the antiretroviral agent to prevent infections.

3. Controlled Delivery of a Birth Control Agent

In one prophetic example, an osmotic pump is formulated for controlled delivery of a birth control agent for up to 7 days for prevention against unwanted pregnancy. In one aspect, the birth control agent is selected from the class of progestins, for example, levonorgestrel. The birth control agent is blended with a gel-forming polymer and compressed into a pellet. The membrane in this example has adequate water vapor transmission rate to allow quick hydration of the pellet. A continuous controlled therapeutically effective dose of the birth control agent is delivered in the vaginal tract for 7 days. Upon release of contents, the membrane loses its integrity and breaks into small fragments that are eliminated from the vaginal tract.

4. Peri-Coital Delivery of a Spermicide

In one prophetic example, an osmotic pump for peri-coital delivery of a spermicide is formulated. The spermicide is blended with a gel-forming polymer and compressed into a pellet. The membrane in this example is pH sensitive and has adequate water vapor transmission rate to allow quick hydration of the pellet. This membrane instantaneously dissolves due to pH change upon introduction of semen in the vaginal tract and releases high neutralizing doses of the spermicide to prevent pregnancy.

5. Controlled Delivery of a Lubricant

In one prophetic example, an osmotic pump is formulated for controlled delivery of a lubricant for up to 1 day for vaginal dryness. The lubricant in this system is a hyper-osmotic solution or gel that creates an osmotic gradient that force extracellular fluids in the vaginal tract to overcome dryness. The hyper-osmotic solution is formed from a high molecular weight hydrophilic polymer and a gel-forming polymer compressed into a pellet and enclosed with a semi-permeable membrane. The membrane in this example has adequate water vapor transmission rate to allow quick hydration of the pellet. Upon release of contents, the membrane loses its integrity and breaks into small fragments that are eliminated from the vaginal tract.

In one aspect, the semi-permeable membrane has porogens or pore-forming agents, for example, salts of sodium or potassium and mono- and di-saccharides that dissolve upon hydration and increase the porosity of the membrane. In such cases, there is minimum lag time after insertion and rapid release of lubricant occurs from the osmotic pump.

In one example, the lubricant is a liquid or gel microencapsulated in a hydrophilic polymer and then compressed with a gel-forming polymer into a pellet. In this aspect, compression forces are strong enough to form a pellet that withstands the coating process but do not deform or disrupt the lubricant microcapsules.

In one example, the lubricant containing pellet may be enclosed in a pH responsive semi-permeable membrane. In this aspect, a controlled dose of lubricant is released initially and a bolus dose is delivered upon membrane dissolution due to semen-induced pH change.

6. Delivery of Polymers and Proteins

In one prophetic example, an osmotic pump is formulated for controlled delivery of macromolecules, such as polymers and proteins, for up to 7 days. The macromolecule is blended with a gel-forming polymer and compressed into a pellet. The membrane in this example has adequate water vapor transmission rate to allow quick hydration of the pellet. A continuous controlled therapeutically effective dose of the macromolecule is delivered in the vaginal tract for 7 days. Upon release of contents, the membrane loses its integrity and breaks into small fragments that are eliminated from the vaginal tract.

7. Simultaneous Delivery of a Microbicde and a Contraceptive

In one prophetic example, an osmotic pump is formulated for controlled delivery of two or more drugs, such as a microbicide and a contraceptive for 7 days for prevention against sexually transmitted infections and unwanted pregnancy. In one aspect, a microbicide from the class of HIV-1 entry inhibitors and a contraceptive from the class of progestins, for example, levonorgestrel, is selected. The drugs are blended with a gel-forming polymer and compressed into a pellet. The membrane in this example has adequate water vapor transmission rate to allow quick hydration of the pellet. The osmotic pump delivers controlled therapeutically effective doses of the drugs in the vaginal tract for 7 days. Upon release of contents, the membrane loses its integrity and breaks into small fragments that are eliminated from the vaginal tract.

In one example, the pellet containing the drugs may be enclosed in a pH responsive semi-permeable membrane. In this aspect, a controlled dose is released initially and a bolus dose is delivered upon membrane dissolution due to semen-induced pH change.

In one example, a microbicide and a contraceptive may be individually blended with gel-forming polymers and compressed into two separate pellets. These two pellets are then recompressed together to form a single structure that is enclosed in a pH sensitive semi-permeable membrane and an orifice is drilled on each flat face to allow drug release.

8. Delivery of Agents for Cervical Ripening

In one prophetic example, an osmotic pump is formulated for controlled delivery of progestin, misoprostol or dinoprostone for 1-2 days for cervical ripening. The drug is blended with a gel-forming polymer and compressed into a pellet. The membrane in this example is pH sensitive has adequate water vapor transmission rate to allow quick hydration of the pellet. A controlled therapeutically effective dose of the progestin is delivered in the vaginal tract until sufficient dilation is achieved. If necessary, the vaginal tract is flushed with phosphate buffered saline pH 7.4 to dissolve the pH sensitive semi-permeable membrane and immediately release contents.

9. Controlled Delivery of an Antiviral Agent for Prevention and Treatment Against HSV Infections

In one prophetic example, an osmotic pump is formulated for controlled delivery of an anti-HSV agent for a maximum of 7 days for prevention and treatment of genital HSV infections. The drug is blended with a gel-forming polymer and compressed into a pellet. The membrane in this example has adequate water vapor transmission rate to allow quick hydration of the pellet. A continuous controlled therapeutically effective dose of the anti-HSV agent is delivered in the vaginal tract for 7 days. Upon release of contents, the membrane loses its integrity and breaks into small fragments that are eliminated from the vaginal tract.

In one example, the drug-containing pellet may be enclosed in a pH responsive semi-permeable membrane. In this aspect, a controlled dose of is released initially and a bolus dose is delivered upon membrane dissolution due to semen-induced pH change.

10. Treatment of Bacterial Vaginosis

In one prophetic example, an osmotic pump is formulated for controlled delivery of an antibacterial agent for a maximum of 7 days for prevention and treatment of bacterial vaginosis. The drug is blended with a gel-forming polymer and compressed into a pellet. The membrane in this example has adequate water vapor transmission rate to allow quick hydration of the pellet. A continuous controlled therapeutically effective dose of the antibacterial agent is delivered in the vaginal tract for 7 days. Upon release of contents, the membrane loses its integrity and breaks into small fragments that are eliminated from the vaginal tract.

In one example, the drug-containing pellet may be enclosed in a pH responsive semi-permeable membrane. In this aspect, a controlled dose of is released initially and a bolus dose is delivered upon membrane dissolution due to semen-induced pH change

11. Probiotic Delivery for Restoration and Maintenance of Vaginal Microflora

In one prophetic example, an osmotic pump is prepared as described in Prophetic Example 2 containing probiotic agents, for example spores of several Lactobacilli species or proteins (i.e., lysozyme, lactoferrin, and lactoperoxidase), for restoration of normal vaginal microflora. In one aspect, the osmotic pump is formulated for delivery of oligosaccharides and polysaccharides, for example starches, mannan oligosaccharides, trans-galacto-oligosaccharide, inulin, and fructo-oligosaccharide, for maintenance of normal vaginal microflora. The drug is blended with a gel-forming polymer and compressed into a pellet. The membrane in this example has adequate water vapor transmission rate to allow quick hydration of the pellet. A continuous controlled therapeutically effective dose of the probiotic agent is delivered in the vaginal tract for 7 days. Upon release of contents, the membrane loses its integrity and breaks into small fragments that are eliminated from the vaginal tract.

12. Delivery of Estrogens for Alleviating Vaginal Dryness

In one prophetic example, an osmotic is prepared as described in Prophetic Example 2, whereby the contraceptive is replaced by estrogen for relieving symptoms of vaginal dryness and atrophy in postmenopausal women. The drug is blended with a gel-forming polymer and compressed into a pellet. The membrane in this example has adequate water vapor transmission rate to allow quick hydration of the pellet. A continuous controlled therapeutically effective dose of the estrogen is delivered in the vaginal tract for 7 days. Upon release of contents, the membrane loses its integrity and breaks into small fragments that are eliminated from the vaginal tract.

13. Treatment of Vaginal Yeast Infections, Vaginitis Induced by Candida Infections, or Vulvovaginal Candidiasis

In one prophetic example, an osmotic pump is formulated for controlled delivery of an antifungal agent for a maximum of 7 days. The drug is blended with a gel-forming polymer and compressed into a pellet. The membrane in this example has adequate water vapor transmission rate to allow quick hydration of the pellet. A continuous controlled therapeutically effective dose of the drug is delivered in the vaginal tract for 7 days. Upon release of contents, the membrane loses its integrity and breaks into small fragments that are eliminated from the vaginal tract.

14. Management of Endometriosis

In one prophetic example, an osmotic pump is formulated for controlled delivery of leuprolide acetate for a maximum of 7 days for treatment of endometriosis. In one aspect, leuprolide acetate and a progestogen, for example, norethindrone are delivered simultaneously. A continuous controlled therapeutically effective dose is delivered in the vaginal tract for 7 days. Upon release of contents, the membrane loses its integrity and breaks into small fragments that are eliminated from the vaginal tract.

15. Management of Uterine Fibroids

In one prophetic example, an osmotic pump is formulated for controlled delivery of a selective estrogen receptor modulator for a maximum of 7 days for treatment of uterine fibroids. A continuous controlled therapeutically effective dose is delivered in the vaginal tract for the duration. Upon release of contents, the membrane loses its integrity and breaks into small fragments that are eliminated from the vaginal tract.

16. Topical Bromocriptine Delivery

In one prophetic example, an osmotic pump is formulated for controlled delivery of bromocriptine for treatment of hyperprolactinemia for a maximum of 7 days. A continuous controlled therapeutically effective dose is delivered in the vaginal tract for 7 days. Upon release of contents, the membrane loses its integrity and breaks into small fragments that are eliminated from the vaginal tract.

### G. REFERENCES

Adams, J. L., Kashuba, A. D. (2012) Formulation, pharmacokinetics, and pharmacodynamics of topical microbicides. Best Pract. Res. Clin. Obstet. Glynaecol. 26: 451-462.
Amidon, G. L., Lennernas, H., Shah, V. P., Crison, J. R. (1995) A theoretical basis for a biopharmaceutic drug classification: the correlation of in vitro drug product dissolution and in vivo bioavailability. Pharm. Res. 12: 413-420.
Buckheit Jr., R. W., Hartman, T. L., Watson, K. M., Chung, S. G., Cho, E. H. (2008) Comparative evaluation of the inhibitory activities of a series of pyrimidinedione congeners that inhibit human immunodeficiency virus types 1 and 2. Antimicrob. Agents Chemother. 52: 225-236.
Clark, M. R., Aliyar, H. A., Lee, C. W., Jay, J. I., Gupta, K. M., Watson, K. M., Stewart, R. J., Buckheit, R. W., Kiser, P. F. (2011) Enzymatic triggered release of an HIV-1 entry inhibitor from prostate specific antigen degradable microparticles. Int. J. Pharm. 413: 10-18.
Crawford, R. R., Esmerian, O. K. (1971) Effect of plasticizers on some physical properties of cellulose acetate phthalate films. J. Pharm. Sci. 60: 312-314.
Daugherity, P., Nause, R. (2009) Cellulose acetate. In: Rowe, R. C., Sheskey, P. J., Quinn, M. E. (Eds.), Handbook of Pharmaceutical Excipients, 6^{th} Ed. Pharmaceutical Press, London, UK, pp. 141-143.
Daugherity, P., Nause, R. (2009) Cellulose acetate phthalate. In: Rowe, R., Sheskey, P., Quinn, M. (Eds.), Handbook of Pharmaceutical Excipients, 6^{th} Ed. Pharmaceutical Press, London, UK, pp. 143-146.
Grabovac, V., Guggi, D., Bernkop-Schnurch, A. (2005) Comparison of the mucoadhesive properties of various polymers. Adv. Drug Deliv. Rev. 57: 1713-1723.
Gupta, K. M., Barnes, S. R., Tangaro, R. A., Roberts, M. C., Owen, D. H., Katz, D. F., Kiser, P. F. (2007) Temperature and pH sensitive hydropolymers: an approach towards smart semen-triggered vaginal microbicidal vehicles. J. Pharm. Sci. 96: 670-681.
Herrlich, S., Spieth, S., Messner, S., Zengerle, R. (2012) Osmotic micropumps for drug delivery. Adv. Drug Deliv. Rev. 64: 1617-1627.
Johnson, T. J., Srinivasan, P., Albright, T. H., Watson-Buckheit, K., Rabe, L., Martin, A., Pau, C. P., Hendry, R. M., Otten, R., McNicholl, J., Buckheit Jr., R., Smith, J., Kiser, P. F. (2012) Safe and sustained vaginal delivery of pyrimidinedione HIV-1 inhibitors from polyurethane intravaginal rings. Antimicrob. Agents Chemother. 56: 1291-1299.
Malaterre, V., Ogorka, J., Loggia, N., Gurny, R. (2009). Oral osmotically driven systems: 30 years of development and clinical use. Eur. J. Pharm. Biopharm. 73: 311-323.
Marrazzo J, Ramjee G, Nair G, Palanee T, Mkhize B, Nakabiito Taljaard M, Piper J, Gomez Feliciano K, M., C. (2013) Pre-exposure prophylaxis for HIV in women: daily oral tenofovir, oral tenofovir/emtricitabine or vaginal tenofovir polymer in the VOICE study (MTN 003). 20th Conference on Retroviruses and Opportunistic Infections, Atlanta, Ga., pp. 26 LB.
Minkin, M. J., Maamari, R., Reiter, S. (2013) Improved compliance and patient satisfaction with estradiol vaginal tablets in postmenopausal women previously treated with another local estrogen therapy. Int. J. Womens Health 5: 133-139.
Rioux, J. E., Devlin, C., Polymerfand, M. M., Steinberg, W. M., Hepburn, D. S. (2000) 17beta-estradiol vaginal tablet versus conjugated equine estrogen vaginal cream. Menopause 7: 156-161.
Royce, R. A., Sena, A., Cates Jr., W., Cohen, M. S. (1997) Sexual transmission of HIV. N. Engl. J. Med. 336: 1072-1078.
Sprockel, O. L., Prapaitrakul, W., Shivanand, P. (1990) Permeability of cellulose polymers: water vapour transmission rates. J. Pharm. Pharmacol. 42: 152-157.
Swamy, G. K. (2012) Current methods of labor induction. Semin. Perinatol. 36: 348-352.
Theeuwes, F. (1975) Elementary osmotic pump. J. Pharm. Sci. 64: 1987-1991.
Theeuwes, F., Yum, S. I. (1976) Principles of the design and operation of generic osmotic pumps for the delivery of semisolid or liquid drug formulations. Ann. Biomed. Eng. 4: 343-353.
Zhang, T., Zhang, C., Agrahari, V., Murowchick, J. B., Oyler, N. A., Youan, B. B. (2013) Spray drying tenofovir loaded mucoadhesive and pH-responsive microspheres intended for HIV prevention. Antiviral Res. 97: 334-346.

### H. EXAMPLES

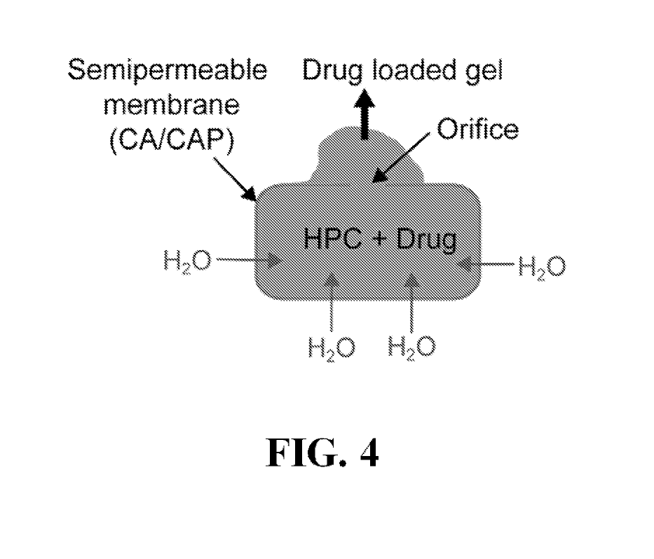

The underlying physics behind osmotic pump technology has not changed despite the large amount of work on the design and application in oral and implantable drug delivery (Theeuwes, F. (1975) J. Pharm. Sci. 64:1987-1991). Drug release rate from these systems is typically a function of rate of water entry into the device due to an osmotic pressure gradient between the device core and the environment (Theeuwes and Yum (1976) Ann. Biomed. Eng. 4:343-353). The osmotic pressure difference can be controlled by the nature and concentration of the osmotic agent, the water vapor transmission rate (P) of the semipermeable membrane (SPM) and geometry of the drug delivery orifices (FIG. 4). An interplay between these properties, aids in achieving controlled zero order drug release for timed durations. Unless inserted as implants, osmotic pumps are currently utilized for 24-h controlled oral delivery (Herrlich, S., et al. (2012) Adv. Drug Deliv. Rev. 64: 1617-1627; Malaterre, V., et al. (2009) Eur. J. Pharm. Biopharm. 73: 311-323). The reason for the lack of reports on osmotic tablets for vaginal delivery is uncertain, but perhaps researchers assumed there was limited fluid to drive the drug release. Therefore, to achieve multiple day drug delivery, the osmotic pump tablet (OPT) was modified to ensure retention in the vaginal tract for extended durations by designing an OPT coated with a bioadhesive polymer (Grabovac, V., et al. (2005) Adv. Drug Deliv. Rev. 57: 1713-1727) that delivers a viscous polymer. The polymer is hypothesized to aid in retaining the formulation in the vaginal canal and may improve drug distribution in the vaginal tract. This approach utilized a semi-solid polymer gel-forming polymer, hydroxypropyl cellulose (HPC) as the osmoattractant core instead of NaCl or polyethylene glycol. The high molecular weight of this gel-forming polymer is unlikely to cause osmotic stress to mucosal tissues unlike other systems that have high salt concentrations. Water is driven into the HPC core resulting in polymer swelling and extrusion of a vaginal polymer through the orifice and delivery of drug in the vaginal canal.

Herein, the fabrication of a multi-day intravaginal OPT for delivery of IQP-0528, a pyrimidinedione, with potent nanomolar non-nucleoside reverse transcriptase and entry inhibition activity (Buckheit, Jr., R. W., et al. (2008) Antimicrob. Agents Chemother. 52: 225-236; Johnson, T. J., et al. (2012) Antimicrob. Agents Chemother. 56: 1291-1299), is described, and along with its evaluation in the sheep vaginal model. Vaginal fluid samples were collected using a new multiswab device to determine spatial drug distribution by placing several circular sponges at precise positions along a penis shaped rod. This allows for the mapping of drug distribution in the vaginal tract as a function of time and distance.

The following examples are put forth so as to provide those of ordinary skill in the art with a complete disclosure and description of how the compounds, compositions, articles, devices and/or methods claimed herein are made and evaluated, and are intended to be purely exemplary of the invention and are not intended to limit the scope of what the inventors regard as their invention. Efforts have been made to ensure accuracy with respect to numbers (e.g., amounts, temperature, etc.), but some errors and deviations should be accounted for. Unless indicated otherwise, parts are parts by weight, temperature is in ° C. or is at ambient temperature, and pressure is at or near atmospheric.

1. Experimental Methods

a. Osmotic Pump Tablet Fabrication

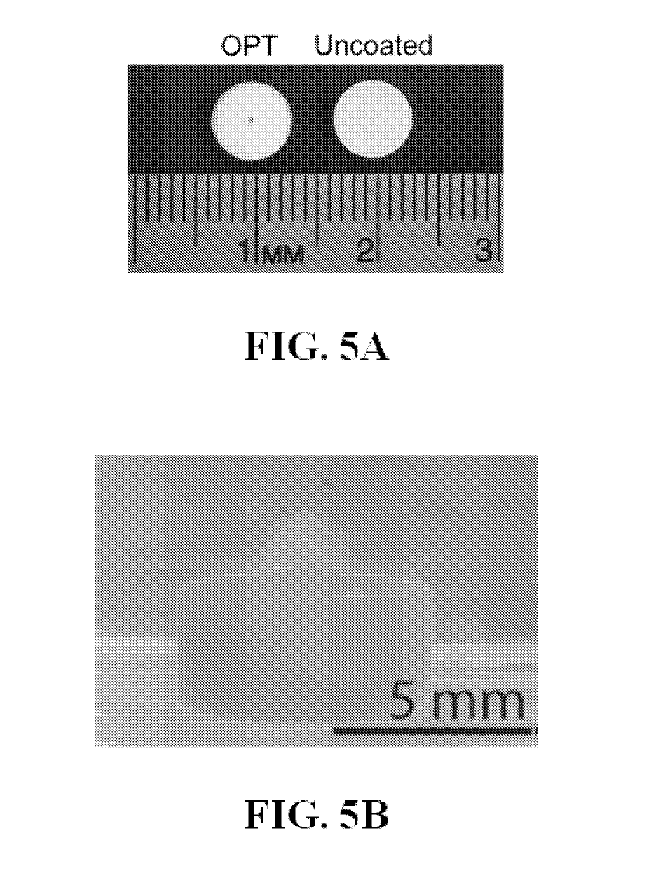

Vaginal tablets with 10 wt % IQP-0528 in HPC matrix were fabricated using a standard pellet press, spray-coated with SPM gel-forming polymers, cellulose acetate phthalate (CAP) (Daugherity and Nause (2009) Handbook of Pharmaceutical Excipients, 6^{th} Ed. Pharmaceutical Press, London, UK, pp. 143-146) or cellulose acetate (CA) (Daugherity and Nause (2009) Handbook of Pharmaceutical Excipients, 6^{th} Ed. Pharmaceutical Press, London, UK, pp. 141-143) and a drug delivery orifice was manually drilled (FIGS. 5A and 5B). CAP, an enteric coating polymer, is insoluble at the approximate vaginal pH 4, but is soluble at seminal pH of 6-8 (Daugherity and Nause (2009) Handbook of Pharmaceutical Excipients, 6^{th} Ed. Pharmaceutical Press, London, UK, pp. 143-146), a feature which was utilized for engineering a semen-triggered burst drug release system.

Briefly, IQP-0528 (1-(cyclopropylmethyl)-6-(3,5-dimethylbenzoyl)-5-isopropylpyrimidine-2,4(1H,3H)-dione; ImQuest Biosciences, Frederick, Md.) and hydroxy propylcellulose (HPC GF; Klucel Pharm, Hercules, Wilmington, Del.; MW=370 kDa) were mixed using a homogenizer (Qiagen Tissuelyser II, Valencia, Calif.) for 9 min at 30 Hz to achieve approximately 10 wt % IQP-0528 loading. Pellets were formed by compression on a manual bench top press (Carver, Wabash, Ind.) at 2 metric tons for 20 s using a punch and die set specifically designed for 6.35 mm (¼″) diameter pellets (Carver, Wabash, Calif.). Pellets were coated with 5 wt % solutions of cellulose acetate phthalate (CAP; Sigma-Aldrich, St. Louis, Mo.; MW=2534.12 Da) or cellulose acetate (CA; Acros Organics, Thermo Fisher Scientific, Geel, Belgium; MW=100 kDa) in acetone using an in-house fabricated spray-coater for 20-25 minutes. Pellets were massed and allowed to dry under vacuum until constant mass at rt was achieved. An orifice was made on one of the flat sides manually using a 20 G needle and the diameter was determined microscopically at 4× magnification. Membrane thickness of approximately 150±20 μm and orifice diameter of 800±80 μm was maintained to ensure uniformity between batches. To understand the working of the CAP-OPT, HPC pellets with 1 wt % Quinoline Yellow SS (Spectrum Chemicals, Gardena, Calif.) were prepared and coated with 0.1 wt % Rhodamine B (Sigma Aldrich, St. Louis, Mo.) in CAP similar to the drug loaded OPTs.

b. In Vitro IQP-0528 Release

In vitro IQP-0528 release was tested under simulated vaginal conditions (2 wt % Solutol® HS (BASF Corporation, Ludwigshafen, Germany) in 25 mM acetate buffer pH 4.2) at 37° C. and 80 rpm shaking for 10 days. Each OPT was placed in 20 mL release media and whole media was replaced daily to maintain sink conditions and analyzed by reverse phase high pressure liquid chromatography (HPLC).

Alternatively, the osmotic pump was placed in 10 mL 25 mM acetate buffer (pH 4.2) for 96 h. Release media was changed periodically at t=1, 2, 5, 8, 25, 34, 51, 80, and 96 h and replaced with fresh buffer. After 96 h the acetate buffer was replaced with 15 mL phosphate buffered saline pH 7.4 to study pH sensitivity of the system. The pH of the system was found to by pH 7.5. Samples were analyzed using a high performance liquid chromatography (HPLC) system on a Zorbax ODS column (4.6×250 mm, 5 μm) using acetonitrile (65%) and water (35%) at a flow rate of 1.5 mL/min at 267 nm. Data was calculated using a calibration curve created by computing the peak areas obtained using differing active pharmaceutical ingredient (API) standards in methanol with the above HPLC method.

C. Multiswab Fabrication

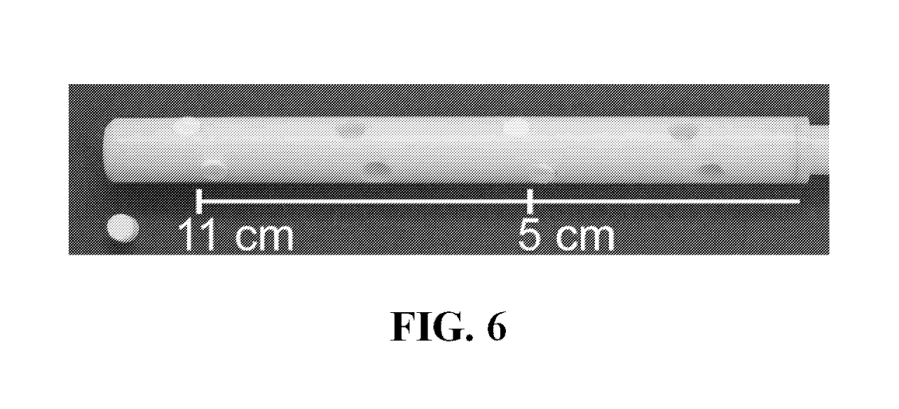

Multiswabs were fabricated on a lathe and mill from ½″ acrylonitrile butadiene styrene rod (McMaster-Carr, Robbinsville, N.J.). Orifices to hold the multiswab sponges were made with a square end mill to an outer diameter of 5 mm and 4 mm deep. Pairs of such orifices on opposing sides were spaced longitudinally 2, 5, 8 and 11 cm from the handle end representing the length from the introitus during application. Additional pairs, rotated 45° with respect to the first, were milled on opposing sides with 0.5 mm offset towards the handle. The leading edge was rounded and a handle fashioned at a reduced diameter and approximately 5 cm long. (FIG. 6)

d. In Vivo Studies

All animals were housed at the Center for Comparative Medicine, University of Utah, Utah. All procedures were conducted under approved IACUC protocols in accordance with the standards incorporated in the Guide for the Care and Use of Laboratory Animals (National Research Council of the National Academies, 2010). IQP-0528 pharmacokinetics from uncoated tablets (N=3) and CA-OPT (N=5) was evaluated in adult, Columbian cross-bred ewes for 2 or 10 days respectively. Briefly, 2 tablets or OPTs were administered using a custom-designed system composed of an applicator made using an 18 cm long Tygon® tubing (3606; ID ¼″, OD 5/16″; Cole Parmer, Vernon Hills, Ill.) and a plunger with a flatten end. The applicator was inserted to a depth of 15 cm and the plunger used to expel tablets into the vaginal tract. Vaginal fluid was collected using a multiswab device (FIG. 6), with swabs (5 mm diameter swabs cut from Ultracell® Nasal packs, Beaver Visitec International, Waltham, Mass.) inserted at 11 and 5 cm from the introitus. Samples were collected at day 0 (baseline) and various time-points during the study, 6 h and days 1 and 2 for uncoated and 6 h and days 1, 2, 3, 5, 7 and 10 for OPTs.

Drug was extracted from the swabs using IQP-0532, a congener of IQP-0528 (Buckheit Jr., R. W., et al. (2008) Antimicrob. Agents Chemother. 52, 225-236) as the internal standard and methanol:water (4:1) as extractant solution. Briefly, 200 μL of the extractant was added on the swab, allowed to equilibrate for 1 h at room temperature and the contents transferred to an Ultrafree® Centrifugal filter (Durapore®-PVDF, 0.65 μm, Merck Millipore, Billierica, Mass.) and centrifuged at 16,000×g for 10 min. The process was repeated twice to maximize drug extraction. For standards, known volume of drug stock solutions were incubated with swabs and extracted as above. Samples were analyzed using an Agilent LC-single quad MS fitted with an atmospheric pressure chemical ionization-electrospray ionization (APCI-ESI) multimode. The lower limit of quantification (LLOQ) for IQP-0528 calculated using average fluid mass for all samples as 0.1 μg/mL.

2. In Vitro Release Studies

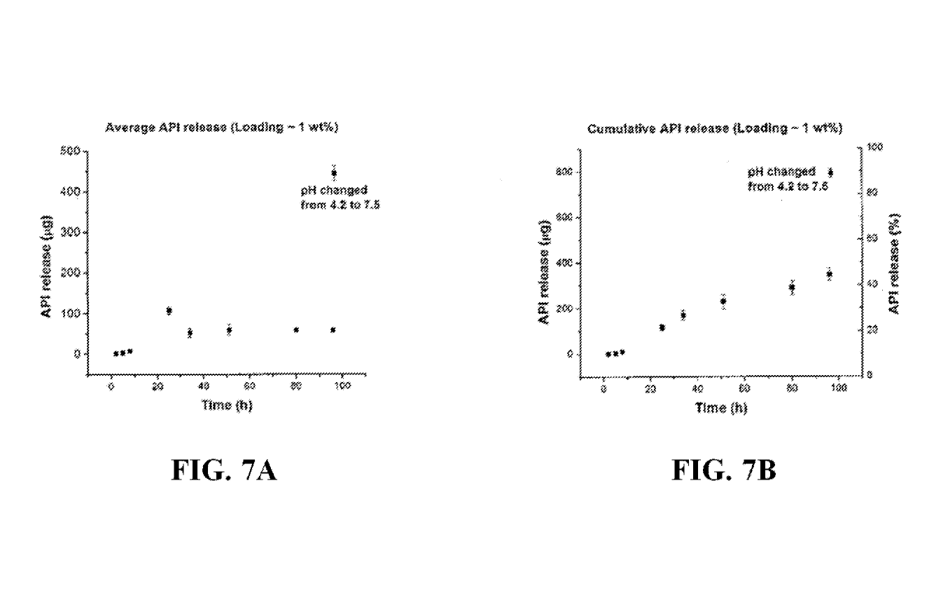

The daily and cumulative release of the API from the formulation is shown in FIGS. 7A and 7B, respectively. Approximately 120 μg of the drug was released at 24 h. Upon equilibrium, 50 μg of the drug was released up to 96 h which accounts for 45% of the total drug load. The change in pH from 4.2 to 7.5 resulted in completed dissolution of the tablet with 450 μg of the drug being released.

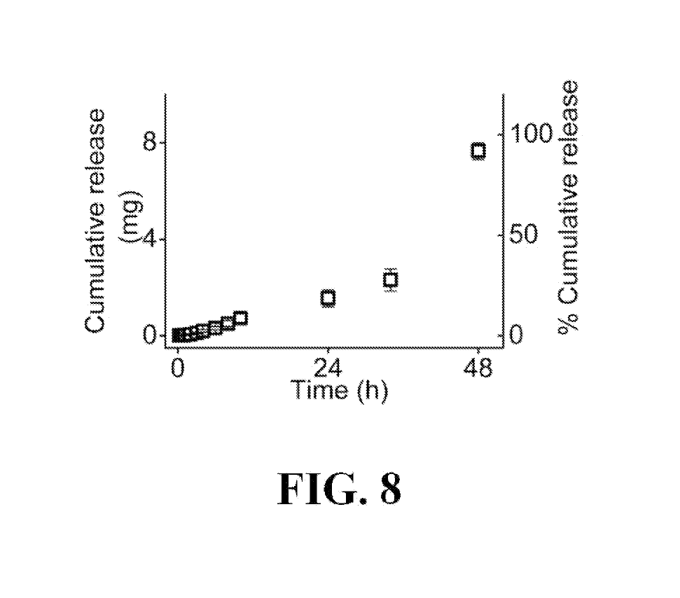

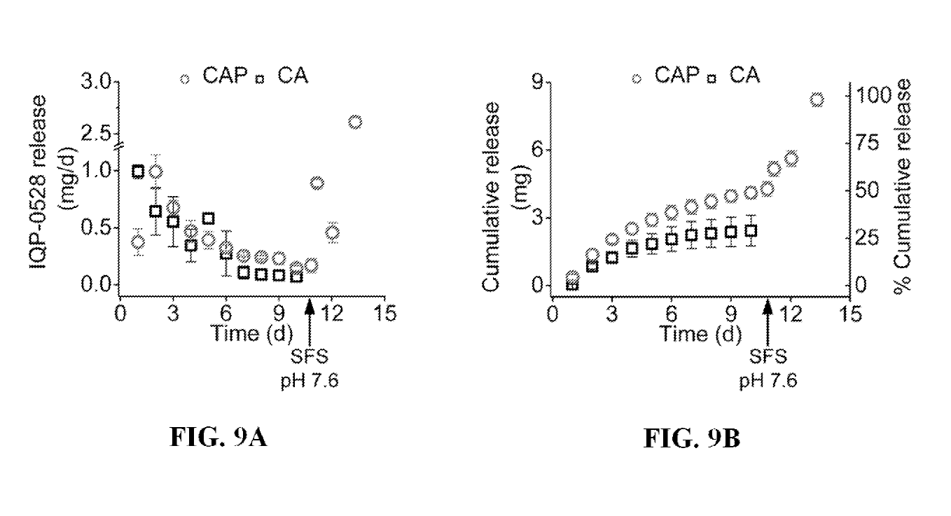

To study the advantage of an OPT over conventional tablet formulations, an identical core composition was maintained for IQP-0528 pellets (FIG. 5A; 8.4±0.6 wt %, N=10). As expected, uncoated tablets exhibited faster swelling with almost complete drug dissolution (91.8±4.1% (N=5) in 48 h; FIG. 8). In contrast, OPTs showed multiday IQP-0528 release in vitro. Slow drug release was noted early on with 0.9±0.06 mg (10.7±0.7%) and 0.4±0.09 mg (4.6±1%) drug released on day 1 from CA- and CAP-OPTs, respectively. A total of 29.5±8.4% (N=4) and 47.1±3.3% (N=6) [P=0.0015] of the drug load was delivered by day 10 from CA- and CAP-OPTs, respectively (FIGS. 9A and 9B). The variation in the amount of drug released was significantly different and can be attributed to the difference in the water vapor transmission rate (P) from the two membranes. Since membrane thickness and area and orifice area were constant for both the OPTs, the membrane permeability will control water uptake by the core and resulting drug release (P_{CAP}=0.52 g m⁻²/24 h or 0.002 mg cm⁻² h⁻¹ and P_{CA}=5 mg cm⁻² h⁻¹ (Crawford and Esmerian (1971) J. Pharm. Sci. 60: 312-314; Sprockel, O. L., et al. (1990) Pharm. Pharmacol. 42: 152-157). Although the data indicated that release could have continued for more than 10 days, it is postulated that short to extended duration delivery systems that last on the order of 2-5 days will be of the most utility in the HIV prevention field.

3. pH Sensitivity

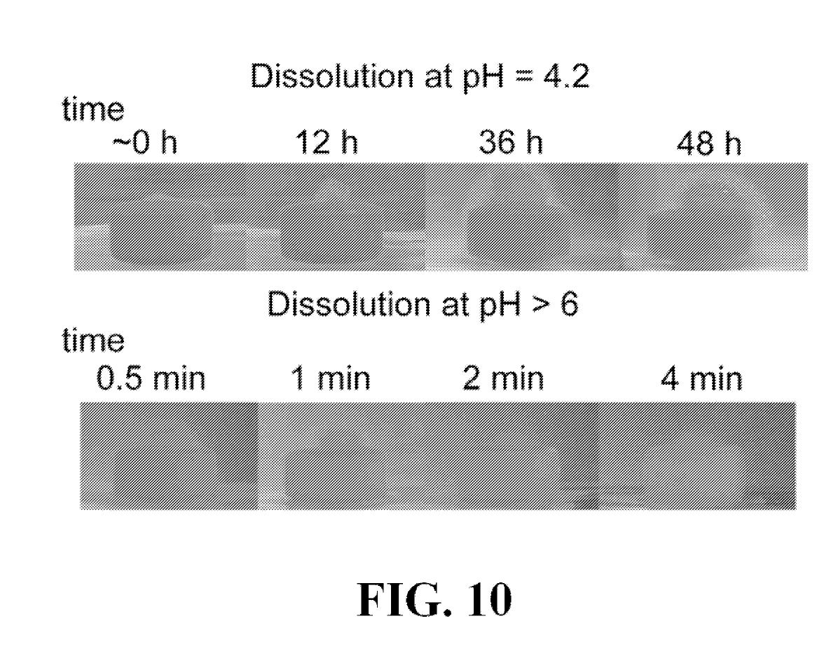

Semen can cause a large pH change in the vaginal canal that can be used to design semen triggered release of drugs (Clark, M. R., et al. (2011) Int. J. Pharm. 413: 10-18; Gupta, K. M., et al. (2007) J. Pharm. Sci. 96: 670-681; Zhang, T., et al. (2013) Antiviral Res. 97: 334-346). To test the suitability of the CAP-OPT as a semen-triggered DDS the release media was changed from acetate buffer pH 4.2 (simulated vaginal conditions) to seminal fluid simulant pH 7.6 on day 10. Dissolution of the CAP coating occurred within minutes of the pH change, exposing a polymer core followed by 49% of the drug load being delivered in the next 2.5 days amounting to 97% total drug release (FIG. 9B). To visualize the working of the CAP-OPT, a yellow dye-loaded HPC core was formulated with a rhodamine B-CAP coating (FIG. 10). A bright yellow polymer labeled with the dye was seen to extrude out of the drug delivery orifice under osmotic forces when the system was placed in media (pH 4.2; FIG. 6). Change in pH to >6 resulted in rapid dissolution of the CAP SPM, exposing the hydrated polymer core.

4. Vaginal Distribution in a Sheep Model

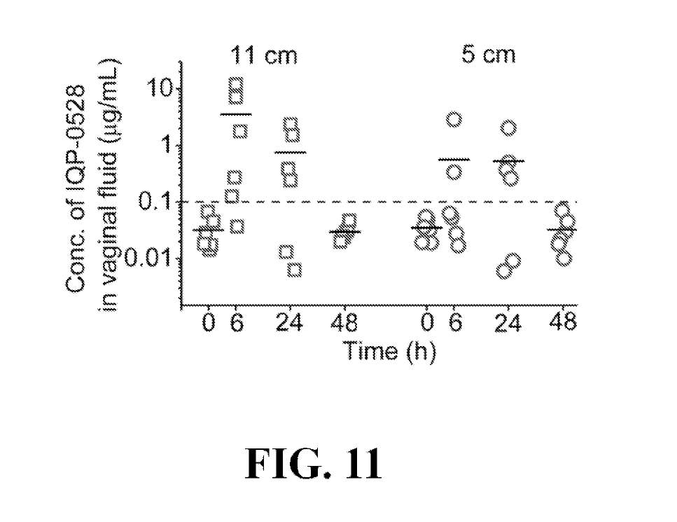

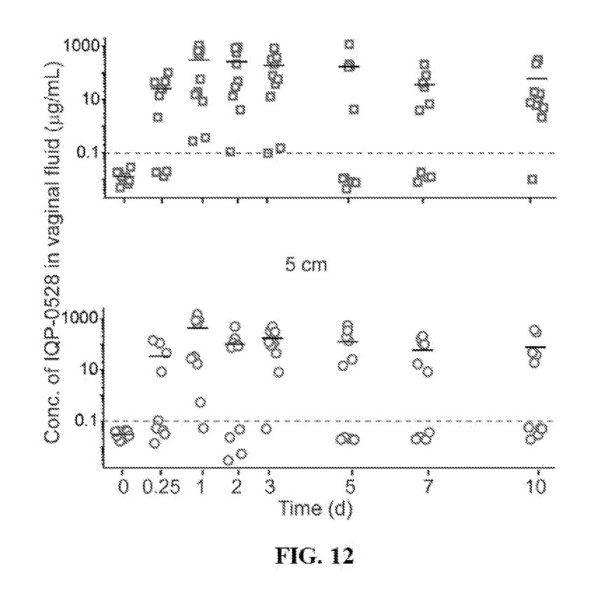

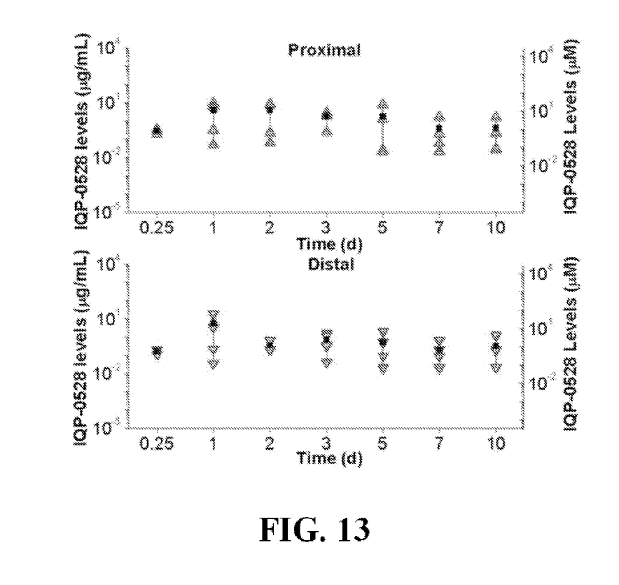

IQP-0528 vaginal distribution from uncoated tablets (N=3) and CA-OPT (N=5) was evaluated in a sheep model. Due to lack of a convenient animal model where vaginal pH is in the acidic range, pH triggered release from the CAP-OPT was unable to be studied in vivo. Spatially registered swabs were collected at 11 and 5 cm from the introitus using the multiswab device (FIG. 6). Intravaginal administration of 2 uncoated tablets per animal resulted in Cₘₐₓ of 3.5 μg/mL (mean; range 12-0.04 μg/mL) and 0.6 μg/mL (mean; range 2.9-0.02 μg/mL) distal and proximal to the introitus, respectively, at 6 h (N=3) with no drug detected by 48 h (FIG. 11). In the first CA-OPT study (N=3), only ⅙ OPTs inserted were retained in the first 6 h. This finding is attributed to the small amounts of polymer released early on being insufficient to retain these systems in the canal. Thus to increase retention, the face of the OPT opposite to the orifice was asymmetrically dip-coated with Carbopol 974P (3 wt % in methanol) followed by 24-h vacuum drying and wetted the OPTs before insertion. This CA-OPT formulation resulted in an average IQP-0528 concentration of 145 μg/mL in a 10-day application. The drug was well distributed in the vaginal tract with 152 μg/mL (mean; range 1176.3-0.001 μg/mL) at 11 cm and 137.2 μg/mL (mean; range 1404-0.003 μg/mL) at 5 cm from the introitus. Peak drug levels were noted on day 1, 309.3 μg/mL (mean; range 1087.3-0.3 μg/mL) distal and 420 μg/mL (mean; range 1404-0.05 μg/mL) proximal to the introitus (FIGS. 12 and 13).

It will be apparent to those skilled in the art that various modifications and variations can be made in the present invention without departing from the scope or spirit of the invention. Other embodiments of the invention will be apparent to those skilled in the art from consideration of the specification and practice of the invention disclosed herein. It is intended that the specification and examples be considered as exemplary only, with a true scope and spirit of the invention being indicated by the following claims.

1. A controlled-release device comprising:
(a) a core comprising:
(i) a water-swellable gel-forming polymer; and
(ii) optionally, an osmotic agent; and
(iii) optionally, a pharmacologically active agent;
(b) a substantially inelastic, water-insoluble coating substantially enclosing the core, wherein at least a portion of the coating comprises a semipermeable membrane; and
(c) at least one orifice in the coating, wherein the orifice is positioned and dimensioned to allow controlled-release of the polymer from the core in response to swelling of the polymer,
wherein the core comprises no more than 15 wt % of the osmotic agent.

2. The device of claim 1, wherein the core comprises no more than 5 wt % of the osmotic agent.

3. The device of claim 1, wherein the polymer is selected from a boronic acid-hydroxamic acid crosslinked polymer and a boronic acid-diol crosslinked polymer.

4. The device of claim 1, wherein the polymer is selected from hydroxypropyl cellulose and hydroxyethyl cellulose.

5. The device of claim 1, wherein the pharmacologically active agent is a biologic.

6. The device of claim 5, wherein the biologic is a probiotic.

7. The device of claim 1, further comprising a pH-responsive material covering the core.

8. A controlled-release device comprising:
(a) a core comprising:
(i) a water-swellable gel-forming polymer; and/or an osmotic agent; and
(ii) optionally, a pharmacologically active agent; and
(b) a water-insoluble coating substantially enclosing the core, wherein at least a portion of the coating comprises a pH-responsive material.

9. The device of claim 8, wherein the core comprises no more than 15 wt % of the osmotic agent.

10. The device of claim 8, wherein the polymer is selected from a boronic acid-hydroxamic acid crosslinked polymer and a boronic acid-diol crosslinked polymer.

11. The device of claim 8, wherein the polymer is selected from hydroxy propyl cellulose and hydroxy ethyl cellulose.

12. The device of claim 8, wherein the pharmacologically active agent is a spermicide.

13. A method of preventing fertility in a subject, the method comprising contacting a controlled-release device with mucous membrane tissue, wherein the device comprises:
(a) a core comprising:
(i) a water-swellable gel-forming polymer;
(ii) an effective amount of a pharmacologically active agent; and
(iii) optionally, an osmotic agent;
(b) a substantially inelastic, water-insoluble coating substantially enclosing the core, wherein at least a portion of the coating comprises a semipermeable membrane; and
(c) at least one orifice in the coating, wherein the orifice is positioned and dimensioned to allow controlled-release of the polymer from the core in response to swelling of the polymer,
wherein the core is comprises no more than 15 wt % of the osmotic agent; and wherein the pharmacologically active agent is a birth control agent.

14. The method of claim 13, wherein the tissue is selected from oral, nasal, vaginal, and rectal.

15. A method of preventing fertility in a subject, the method comprising contacting a controlled-release device with vaginal membrane tissue, wherein the device comprises:
(a) a core comprising:
(i) a water-swellable gel-forming polymer and/or an osmotic agent; and
(ii) an effective amount of a pharmacologically active agent;
(b) a substantially inelastic, water-insoluble coating substantially enclosing the core, wherein at least a portion of the coating comprises a semipermeable membrane; and
(c) at least one orifice in the coating, wherein the orifice is positioned and dimensioned to allow controlled-release of the pharmacologically active agent from the core,
wherein the pharmacologically active agent is a birth control agent.
